(19) United States
(12) Patent Application Publication
(10) Pub. No.: US 2015/0247149 A1
(43) Pub. Date: Sep. 3, 2015

(54) Detecting and Treating Liver Damage

(71) Applicant: The Regents of the University of California, Oakland, CA (US)

(72) Inventors: Ariel E. Feldstein, San Diego, CA (US); Akiko Eguchi, San Diego, CA (US)

(21) Appl. No.: 14/607,721

(22) Filed: Jan. 28, 2015

Related U.S. Application Data
(63) Continuation of application No. PCT/US2013/054733, filed on Aug. 13, 2013.
(60) Provisional application No. 61/682,354, filed on Aug. 13, 2012.

(51) Int. Cl. C12N 15/113; C07K 16/40; G01N 33/569

(52) U.S. Cl. CPC C12N 15/1137; G01N 33/56966; C07K 16/40; G01N 2800/085

(57) ABSTRACT

The invention provides a method of detecting, monitoring, assessing and treating non-alcoholic fatty acid liver disease (NAFLD) and associated liver damage in a subject comprising measuring the amount of hepatocyte-derived circulating extracellular vesicles (EVs) and/or microparticle (MPs) in the bodily sample, or the expression level or activity of at least one biomarker expressed or detected in the EVs and/or MPs. The increased amount of EVs or MPs in the bodily sample and/or the increased expression or detection level of the biomarker of interest correlate with the degree or severity of NAFLD, NASH, liver fibrosis, or other associated liver damage, which can be associated with angiogenesis. Prevention and treatment of NAFLD, NASH, liver fibrosis or associated liver damage by reducing EVs or MPs, or targeting the biomarkers expressed in the EVs or MPs are also provided.

### CROSS-REFERENCE TO RELATED APPLICATIONS

This application is a continuation of PCT Application No. PCT/US2013/054733 filed Aug. 13, 2013 which claims priority to U.S. Provisional Application No. 61/682,354 filed Aug. 13, 2012, the entire contents of which are incorporated by reference herewith.

### STATEMENT OF GOVERNMENT INTEREST

This invention was made with government support under Grant Nos. DK076852 and DK082451 awarded by the National Institutes of Health. The government has certain rights in the invention.

### FIELD OF THE INVENTION

The invention relates to a method of detecting, diagnosing, monitoring, assessing and treating the degree or severity of Nonalcoholic Fatty Liver Disease (NAFLD), particularly Nonalcoholic steatohepatitis (NASH) and/or liver fibrosis in a subject. The invention further relates to therapeutic targets for NAFLD, NASH, and other liver damage or diseases.

### BACKGROUND OF THE INVENTION

Nonalcoholic Fatty Liver Disease (NAFLD) is currently the most common form of chronic liver disease affecting both adults and children, and is strongly associated with obesity and insulin resistance [1, 2]. One in three adults and one in ten children or adolescents in the United States have hepatic steatosis, a stage within the spectrum of NAFLD that is characterized by triglyceride accumulation in liver cells and usually follows a benign non-progressive clinical course [3]. Nonalcoholic steatohepatitis (NASH) is defined as lipid accumulation with evidence of cellular damage, inflammation, neovascularization, and different degrees of scarring or fibrosis [4]. NASH is a serious condition as approximately 25% of these patients can progress to cirrhosis and related complications, including portal hypertension, liver failure and hepatocellular carcinoma [5, 6].

Growing evidence suggest that angiogenesis plays a central role in the progression to NASH, particularly the development of fibrosis [7]. Indeed, marked hepatic neovascularization has been reported in patients with NASH, as well as in experimental models of the disease, which parallel the extent of fibrosis present [8-11]. Angiogenesis is a key pathological feature of experimental and human steatohepatitis, now the most common chronic liver disease in the Western world. However, the mechanisms triggering angiogenesis in NASH as well as a number of other chronic liver conditions remain poorly and incompletely understood. Increased expression and release of pro-angiogenic factors such as VEGF by activated Kupffer cells, the resident liver macrophages, has been implicated, likely as the results of local hypoxia [7]. More recently, the degree of angiogenesis in the livers of NASH patients has been shown to tightly correlate with the activation of caspase 3 in hepatocytes [12]. However, the molecular and signaling mechanisms linking lipid accumulation within hepatocytes to angiogenesis and a potential link between lipotoxicity and angiogenesis remain largely unknown. Lipid overloaded hepatocytes may release pro-angiogenic signals that regulate endothelial cell migration and angiogenesis.

Therefore, it is important to identify specific signals released by the injured hepatocytes to communicate with non-parenchymal cells in the liver that initiates the abnormal healing response and inflammation.

### SUMMARY OF THE INVENTION

The invention provides a method of detecting, predicting, monitoring, assessing diagnosing and treating liver damage associated with nonalcoholic fatty acid liver disease (NAFLD) in a subject, comprising: a) obtaining a biological sample of the subject, b) measuring circulating extracellular vesicles (EVs) in the biological sample, and c) deriving a risk score for liver damage by calculating an amount of circulating EVs in the sample relative to circulating EVs in a control dataset from a population of individuals without NAFLD or liver damage associated therewith. An increased amount of the total- or hepatocyte-derived EVs and/or microparticles (MPs) in the subject compared to the control dataset is indicative of a more severe NAFLD and potentially nonalcoholic steatohepatitis. In certain embodiments, the inventive method further comprises algorithmic inclusion of quantitative data from one or more clinical indicia including at least one of the subject's age, body mass index, liver functions. The method can include obtaining a bodily biological sample from subject that includes total- or hepatocyte-derived EVs and/or MPs. The amount and type of EVs and/or MPs is then determined, such as by using flow cytometry (FACS) analysis. A risk score is then derived using a predetermined risk ratio of subject to control. An increased risk score corresponds to a relative assessment of NAFLD and treatment indications for nonalcoholic steatohepatitis.

The invention provides that the increased circulating EVs can be derived from hepatocytes. In certain embodiments, the majority of the circulating EVs are microparticles (MPs) derived from hepatocytes. The invention provides that increased amount of circulating EVs and/or MPs in the bodily sample, can contain pro-angiogenesis factors associated with the degree and/or progression of NAFLD, such as NASH or liver fibrosis, and its associated liver damage or angiogenesis. Treatment indications include reducing and/or blocking the internalization of the circulating EVs or MPs by endothelial cells that blocks angiogenesis and/or reduces the progress of NAFLD resulting in NASH. Therefore, the invention provides that circulating EVs or MPs serve as biomarkers and targets for non-invasive or minimally invasive diagnosis, prognosis, or treatment indications for NAFLD, NASH, liver fibrosis and other liver damage or diseases.

In the other embodiments, the invention provides that certain protein biomarkers involved in molecular function and cellular localization are expressed and detected in circulating EVs or MPs. Exemplary protein biomarkers are listed in Tables 1-4 below. The invention encompasses any biomarkers now or later detected in the hepatocytes EVs or MPs. In certain embodiments, the biomarkers expressed in the EVs or MPs are involved in caspase 3 activation. In one embodiment, such a protein biomarker involved in caspase 3 activation is Vanin-1. The invention provides that Vanin-1 is expressed in the hepatocyte-derived EVs or MPs and regulates caspase 3 activation.

The invention further provides a method of preventing or treating angiogenesis or liver damage in a nonalcoholic steatohepatitis (NASH) subject in need, comprising: administering to said subject an angiogenesis or liver damage inhibiting effective amount of a composition comprising an agent that inhibits at least one biomarker expressed in circulating extracellular vesicles (EVs) or hepatocyte-derived MPs. In certain cases, the biomarker inhibiting agent functions by inhibiting internalization of said EVs or MPs by endothelial cells that otherwise cause activation of pro-angiogenic effects, and thereby prevents angiogenesis and treats liver damage associated with the pro-angiogenic effects of said EVs and/or MPs in the subject. In certain embodiments, the invention provides that the biomarker expressed in the EVs and/or MPs is involved in caspase 3 activation. In one embodiment, such a biomarker is Vanin-1. In certain embodiments, the invention provides that Vanin-1 inhibitors, such as an anti-Vanin-1 antibody and/or a siRNA against the nucleic acid encoding Vanin-1 protein, can block the internalization of the circulating EVs or MPs into the endothelial cells, thus, reducing or preventing the pro-angiogenic effects of said EVs and/or MPs so as to prevent or treat the degree or severity of the NAFLD, NASH, or liver fibrosis or other associated liver damage or diseases. A pharmaceutical composition comprising an effective amount of such an agent of interest and a pharmaceutically acceptable excipient is also encompassed by the invention.

The invention further provides a research tool method of identifying a compound that inhibits at least one biomarker expressed in circulating extracellular vesicles (EVs) or microparticles (MPs) derived from hepatocytes, comprising: a) providing a testing system that expresses said biomarker, and b) identifying a compound that inhibits expression of said biomarker in said testing system. Exemplary protein biomarkers are listed in Tables 1-4 below. In one embodiment, the invention provides a method of screening for a compound is capable of interacting with Vanin-1 protein or its encoded nucleic acid, and/or inhibiting caspase 3 activation, and blocking internalization of the circulating hepatocyte-derived EVs and/or MPs into the endothelial cells, resulting in loss of pro-angiogenic effects of EVs or MPs.

### BRIEF DESCRIPTION OF THE DRAWINGS

FIGS. 1A-1C. Lipid loaded hepatocytes release factors that induced endothelial cell migration and angiogenesis. FIG. 1A Quantification graph of HUVEC tube formation assay after exposure for 6 h to palmitic acid treated HepG2 for 24 h (supernatant) or vesicles-free supernatant obtained by ultracentrifugation. Total tube length (pixel) was measured by Wimtube software. FIG. 1B Quantification graph of Boyden's chamber assay (chemotaxis assay) showing number of migrated HUVECs after 16 h of exposure to palmitic acid treated HepG2 for 24 h (supernatant) or vesicles free supernatant. FIG. 1C Quantification graph of wound healing assay (non-oriented migration) of HUVECs, monitored by confocal time-lapse microscope for up to 48 h. Quantification was performed using the Wimtube software and reported as cell-covered area (%). VEGF (100 ng/ml) was used as positive control and serum-free media as negative control. Values represent mean±S. D. from three independent experiments.* P<0.05; ** P<0.01; ***P<0.001, compared to controls.

FIGS. 2A-2G. Microparticles (MPs) are the main membrane vesicle population released by hepatocytes during exposure to lipotoxic fatty acids. FIG. 2A Flow cytometry analysis was performed to detect and quantify Annexin-V FITC-positive MPs (green peak) released by HepG2 after exposure to palmitic acid and isolated by ultracentrifugation. Presence of hepatocyte-derived MPs was measured in MP-free supernatant (brown peak) and 1% BSA (black peak), which were used as negative controls. FIG. 2B Dynamic light scattering analysis of the size (nm, diameter) of hepatocyte derived MPs released after exposure to palmitic acid. FIG. 2C Transmission electron microscopy (TEM) micrographs of hepatocyte-derived MPs released after palmitic acid treatment. Bar, 500 and 100 nm. FIG. 2D Quantification graph of flow cytometry analysis to quantify Annexin V-positive MPs released by HepG2 treated with 0.25 mM of palmitic acid (PA), stearic acid (SA), oleic acid (OA) or 1% BSA (FFAs control vehicle). FIG. 2E Quantification graph of flow cytometry analysis of the number of Annexin V-positive MPs after co-incubation of HepG2 with a caspase 3 inhibitor. FIG. 2F Quantification graph of flow cytometry analysis to quantify Annexin V-positive MPs released by primary rat hepatocytes treated with 0.25 mM of palmitic acid (PA), stearic acid (SA) and oleic acid (OA) or 1% BSA (FFAs control vehicle). FIG. 2G Quantification graph of flow cytometry analysis of the number of Annexin V-positive MPs after co-incubation of primary rat hepatocytes with a caspase 3 inhibitor. Values represent mean±S.D. from three independent experiments. * P<0.05; ** P<0.01; ***P<0.001, compared to controls.

FIGS. 3A-3D. Endothelial cells tube formation and migration depend on MPs released from fat laden HepG2 cells. FIG. 3A Representative micrographs and corresponding quantification graph of tube formation of HUVECs after exposure to MP-free supernatant or HepG2-derived MPs for up to 6 h. FIG. 3B Representative micrographs and corresponding quantification graph of Boyden's chamber assay (chemotaxis assay) of HUVECs exposed to HepG2-derived MPs, MP-free supernatant and controls. FIG. 3C Representative micrographs and corresponding quantification graph of wound healing assay of HUVECs. FIG. 3D Masson's trichrome representative micrographs of angiogenesis and migration assay in vivo and corresponding quantification graph showing the number of endothelial cells (dark red) migrated into the plug removed from athymic BALB/c nude mice which received a subcutaneous injection of 0.5-1 mL of a Matrigel solution in presence or absence (control) of 100 ng/mL VEGF, MP-free supernatant and HepG2-derived MPs. A 10× or 4× magnifications was used for images. VEGF (100 ng/ml) was used as a positive control and serum-free media as negative control (Control). Values represent mean±S.D. from three independent experiments.* P<0.05; ** P<0.01; ***P<0.001 compared to controls.

FIGS. 4A-4F. Hepatocytes-derived microparticles express VNN1 and are internalized into the endothelial cells. FIG. 4A Western blotting analysis of VNN1 in HepG2 and HepG2-derived MPs released after exposure to 1% BSA (FFA control vehicle), 0.25 mM of saturated (palmitic acid, PA) or unsaturated (oleic acid, OA) FFAs. Controls refer to untreated HepG2 or MP-free supernatant. FIG. 4B Internalization of HepG2-derived PKH26-positive MPs (red) into HUVECs (F-actin fibers, green and nuclei, blue) assessed by indirect immunofluorescence after 1 h and 6 h of incubation with MPs. FIG. 4C TEM micrographs of HUVECs incubated with CTB-HRP conjugate and VNN1-gold antibody conjugate to MPs. CTB-HRP conjugate labeling is shown on the plasma membrane of HUVECs (arrows, upper panel) and VNN1 antibody-gold conjugate is shown on MPs internalized in the HUVECs (arrows, lower panel). Bar, 50 nm. FIG. 4D Representative micrographs and corresponding quantification graph of internalization of MPs assessed by immunofluorescence in HUVECs pre-treated with 10 mM Methyl-β-cyclodextrin (MβCD) for 15 minutes at 37° C. or neutralizing antibody for caveolin-1 (cav-1 nAb) for 30 min. at 37° C. PKH26-positive MPs (red), F-actin fibers of HUVECs (green) and nuclei (DAPI, blue). MP-free supernatant (MP-free sup.) was used as negative control. FIG. 4E Representative micrographs of tube formation and FIG. 4(F) Boyden's chamber assay (chemotaxis assay) of HUVECs pre-treated with Rho-associated kinase inhibitor (Y27632, 10 μM) and with cdc42 siRNA or control siRNA (Ctrl siRNA). Images were acquired by Olympus FV1000 Spectral Confocal with 40× lens and analyzed by using ImageJ. * P<0.05; ** P<0.01; ***P<0.001 compared to controls.

FIGS. 5A-5H. Genetic suppression of VNN1 reduces MP internalization and pro-angiogenic effects on endothelial cells. FIG. 5(A) Western blotting analysis of VNN1 expression in HepG2-derived MPs and HepG2, treated with VNN1 siRNA or control siRNA (Ctrl siRNA) and exposed to 0.25 mM of palmitic acid (PA) for 24 h. FIG. 5(B) Flow cytometry analysis of VNN1-positive MPs isolated from HepG2 exposed to 0.25 mM of palmitic acid for 24 h in presence or absence of VNN1 siRNA. FIG. 5(C) Flow cytometry analysis of Annexin V-positive MPs released from HepG2 treated with VNN1 siRNA or control siRNA and exposed to palmitic acid (PA). FIG. 5(D) Flow cytometry analysis and FIG. 5(E) corresponding quantification graph of number of Calcein/FITC-positive HUVECs exposed to MPs isolated from HepG2 treated with VNN1 siRNA or control siRNA (Ctrl siRNA) and exposed to 0.25 mM of palmitic acid. FIG. 5(F) Internalization of PKH26-positive HepG2-derived MPs or MPs lacking of VNN1 (VNN1−/−) (red) into HUVECs (green), assessed by indirect immunofluorescence. FIG. 5(G) Quantification graph of tube formation and FIG. 5(H) Boyden's chamber assay (chemotaxis) of HUVECs incubated with MPs isolated from HepG2 treated with VNN1 siRNA or control siRNA (Ctrl siRNA) and exposed to palmitic acid (PA). A dose of 100 ng/mL of VEGF was used as positive control and serum-free media as negative control. Values represent mean±S.D. from three independent experiments. * P<0.05; ** P<0.01; ***P<0.001, compared to controls.

FIGS. 6A-6E. Microparticles are released into circulation in a diet-induced NASH murine model. FIG. 6A Flow cytometry analysis of circulating Annexin V-positive MPs isolated from wild-type (WT) and Caspase 3 KO mice (n=5) fed with the MCD or control diet (MCS) for 6 weeks to induce NASH. FIG. 6B Western blotting analysis of VNN1 expression in circulating MPs isolated from MCDor MCS-fed wild-type and Caspase 3 KO mice. The lanes were run on the same gel but were noncontiguous. FIG. 6C TEM representative micrographs of circulating MPs isolated from wild type MCD-fed mice for 6 weeks (left panel) and of MPs localized in liver specimens of MCD-fed mice for 6 weeks (right panel). Space of Disse (SD), microvilli (m), hepatocytes (H), lipid droplet (Ld). FIG. 6D Dynamic light scattering Zetasizer analysis of the size (diameter, nm) of MCD-fed mouse circulating purified MPs. FIG. 6E QPCR of hepatocyte-specific miR-122 level in the circulating MPs isolated from MCD- or MCS-fed mice for 6 weeks. Mean values were normalized to U6 snRNA. Values represent mean±S.D. from three independent experiments. * P<0.05; ** P<0.01; ***P<0.001 compared to controls.

FIGS. 7A-7G. In vivo genetic knockdown of hepatic VNN1 resulted in a drastic decrease of MPs internalization and pro-angiogenic effects on endothelial cells. FIG. 7A QPCR of hepatic VNN1 mRNA expression in mice (n=8 per group) placed on MCD or control diet (MCS) for 6 weeks. MCD-fed mice were treated intravenously with VNN1 siRNA-Invivofectamine Rx, control siRNA (Ctrl siRNA) or PBS (mock) as vehicle. Mean values were normalized to 18S gene. FIG. 7B Western blotting analysis of VNN1 expression in circulating MPs isolated from MCS-fed mice (control diet) and MCD-fed mice treated with VNN1 siRNA, control siRNA or PBS (mock). FIG. 7C Flow cytometry analysis of Annexin V positive circulating MPs collected from MCS-fed mice (control diet) and MCD-fed mice treated with VNN1 siRNA, control siRNA or PBS (mock). FIG. 7D Representative micrograph and FIG. 7E corresponding quantification graph of internalization of MPs isolated from MCS-fed mice (control diet) and MCD-fed mice treated with VNN1 siRNA, control siRNA or PBS (mock). Micrographs show PKH26 positive MPs (red), F-actin fibers of HUVECs (green) and DAPI (nuclei, blue). FIG. 7F Quantification graph of tube formation and FIG. 7G Boyden's chamber assay (chemotaxis) of HUVECs incubated with MPs isolated from MCS-fed mice (control diet) and MCD-fed mice treated with VNN1 siRNA, control siRNA or PBS (mock). A dose of 100 ng/mL of VEGF was used as positive control and serum-free media as negative control. Values represent mean±S.D. from three independent experiments. * P<0.05; ** P<0.01; ***P<0.001, compared to controls.

FIG. 8. HepG2 exposed to different saturated and unsaturated free fatty acids. Representative micrographs of Oil red-O staining for lipid droplets in HepG2 exposed to 1% BSA (control) or 0.25 mM of oleic, palmitic and stearic acid for 24 h. 20× magnification was used for acquisition of the pictures.

FIG. 9. Effect of lipotoxic FFAs is counteracted by non-lipotoxic FFAs. Flow cytometry to quantify Annexin V positive MPs released by HepG2 exposed to 0.25 mM of palmitic acid (PA), oleic acid (OA) and a mixture of PA and OA. Values represent mean±S.D. * P<0.05; ** P<0.01; ***P<0.001 compared to controls.

FIG. 10. Cellular localization and molecular function of proteins from hepatocyte-derived MPs. Proteins obtained by three different proteomics analysis of MPs from HepG2 cells exposed to palmitic acid were organized based on cellular localization (top pie chart) and molecular function (bottom pie chart) according to the GO Consortium. Percentages over the total number of proteins were reported in the pie charts.

FIGS. 11A-11D. MPs released by fat-laden rat primary hepatocytes are potent inducers of angiogenesis. FIG. 11A Representative micrographs of tube formation of HUVECs after exposure up to 6 h to rat primary hepatocytes-derived MPs, MP-free supernatant. FIG. 11B HUVECs total tube length has been measured and reported in the quantification graph. FIG. 11C Representative micrographs of Boyden's chamber assay (chemotaxis) of HUVECs treated with primary hepatocytes-derived MPs or MP-free supernatant for 16 h. A 10× magnification was used for images. FIG. 11D Average of HUVECs migrated into the filter is reported in the graph. VEGF (100 ng/ml) was used as a positive control and serum-free media as negative control (control). Values represent mean±S.D. from three independent experiments. * P<0.05; ** P<0.01; ***P<0.001 compared to controls.

FIGS. 12A-12B. Pro-angiogenic effect of hepatocytes-derived MPs is dose-dependent. HepG2 were treated with 0.25 mM of palmitic acid up to 24 h and MPs were isolated from the supernatant by ultracentrifugation. MPs samples were quantified by BCA protein assay and the concentration was determined. Different doses (50, 125, 250 and 500 μg/mL) of MPs were used for assessing FIG. 12A HUVECs tube formation and FIG. 12B Boyden's chamber assay (chemotaxis). 100 ng/mL of VEGF were used as positive control and serum-free media as negative control. Values represent mean±S.D. * P<0.05; ** P<0.01; ***P<0.001 compared to controls.

FIG. 13. Hepatocyte-derived MPs are detectable into HUVECs tube structures. Representative micrographs of tube formation of HUVECs after exposure to HepG2-derived Calcein positive MP (MPsCalcein) up to 6 hours. HUVECs tube formation was captured by an Olympus FV1000 Spectral Confocal with 20× lens.

FIGS. 14A-14B. Genetic suppression of VNN1 expression on MPs significantly reduced MP-mediated angiogenic response of endothelial cell. HepG2 were exposed to palmitic acid (PA) for 24 h and then treated with VNN1 siRNA or control siRNA (Ctrl siRNA). Representative micrographs of (FIG. 14A) tube formation and (FIG. 14B) Boyden's chamber assay (chemotaxis) of HUVECs treated with hepatocyte-derived MPs, MP-free supernatant (MP-free sup.) for 6 h and 16 h, respectively. A dose of 100 ng/mL of VEGF was used as positive control and serum-free media as negative control. A 4× magnification was used for acquisition of pictures.

FIGS. 15A-15F. Pro-angiogenic effect of hepatocytes-derived microparticles acts through VNN1-dependent internalization. FIG. 15A Flow cytometry of Calcein intensity in HUVECs treated with HepG2-derived Calcein positive MPs (MPsCalcein) or MPs incubated with either VNN1 neutralizing antibody (MPsCalcein+VNN1 nAb) or control neutralizing antibody (GAPDH nAb) for 6 hours. FIG. 15B Quantification graph of number of Calcein/FITC-positive HUVECs exposed to MPCalcein with or without VNN1 nAb were reported in graph; FIG. 15C quantification graph of HUVECs tube formation after exposure for 6 hours to HepG2-derived MPs with or without VNN1 nAb; FIG. 15D quantification graph of Boyden's chamber assay (chemotaxis) of HUVECs after incubation with HepG2-derived MPs with or without VNN1 nAb. FIG. 15E Western blotting analysis of endothelial cells activation markers (ICAM-1 and VCAM-1) after exposure for 6 hours with hepatocytes-derived MPs or MPs pre-incubated with VNN1 nAb. GAPDH was used as loading control. FIG. 15F Glutathione activity assay analysis in HUVECs treated with HepG2-derived MPs with or without VNN1 nAb. Glutathione activity is reported in RFU. 100 ng/mL of VEGF were used as positive control and serum-free media as negative control * P<0.05; ** P<0.01; ***P<0.001, compared to controls.

FIGS. 16A-16B. Proangiogenic effects of VNN1 positive MPs are not mediated by induction of endothelial cell proliferation or modulation of PPAR-γ expression. Fat-laden HepG2-derived MPs were incubated with or without VNN1 neutralizing antibody (VNN1 nAb). BrdU proliferation assay was performed by incubating HUVECs for 16 h with MPs or MPs VNN1 nAb. FIG. 16A. Flow cytometry analysis gating (P3) proliferating-FITC-positive HUVECs. BrdU negative staining was included as negative control. FIG. 16B. QPCR of PPAR-γ mRNA expression after incubation of HUVECs with MPs or MPs VNN1 nAb. Mean values were normalized to the housekeeping gene 18S. 100 ng/mL of VEGF were used as positive control and serum-free media as negative control (control). Values represent mean±S.D. * P<0.05; ** P<0.01; ***P<0.001 compared to controls.

FIGS. 17A-17E. Increase of VNN1 in hepatocytes during lipotoxicity is independent of PPARα and γ. FIG. 17A. QPCR of PPARγ mRNA expression in HepG2 treated with 0.25 mM of palmitic acid (PA) or 1% BSA (FFA control vehicle). FIG. 17B. QPCR for PPARα and FIG. 17C VNN1 mRNA in HepG2 treated with 1% BSA, 0.25 mM of palmitic acid (PA), oleic acid (OA) or palmitic acid/oleic acid mix for 24 h. FIG. 17D. QPCR for PPARα and FIG. 17E VNN1 mRNA in HepG2 exposed to 1% BSA, 0.25 mM of palmitic acid (PA), oleic acid (OA) or palmitic acid/oleic acid mix for 24 h and treated with PPARα siRNA or control siRNA (Ctrl siRNA).

FIGS. 18A-18C. Release of circulating microparticles depends on the stage of NASH. C57/B6 mice (n=5) were placed on the MCD, high fat/high carbohydrates (HF/HCarb) or normal chow diet for 6 weeks. FIG. 18A. Circulating MPs were isolated by centrifugation and Annexin V positive MPs were detected by flow cytometry. FIG. 18B. H-E staining. (C) NAFLD activity score. Values represent mean±S.D. * P<0.05; ** P<0.01; ***P<0.001 compared to controls.

FIGS. 19A-19B. Circulating MPs from mice with NASH stimulate angiogenesis ex vivo. C57/B6 mice were paced on the MCD, high fat/high carbohydrates (HF/HCarb) and normal chow diet for 6 weeks. Platelet-free plasma was harvested and MPs were isolated by centrifugation. MPs and MPs-free supernatant were used to induce HUVECs tube formation and chemotaxis (Boyden's chamber assay) ex vivo. Quantification of (FIG. 19A) HUVECs tube formation (total tube length) and (FIG. 19B) Boyden's chamber assay (chemotaxis) is reported in the graph. A dose of 100 ng/mL of VEGF was used as positive control and serum-free media as negative control (control). Values represent mean±S.D. * P<0.05; ** P<0.01; ***P<0.001 compared to controls.

FIGS. 20A-20B. Liver specific in vivo silencing of VNN1 does not affect VNN1 expression in other tissues. Expression of VNN1 mRNA was assessed in (FIG. 20A) kidney and (FIG. 20B) intestine harvested from MCD-fed mice treated with VNN1 siRNA, control siRNA (Ctrl siRNA) or PBS (Mock) to confirm the liver-specific knockdown of VNN1. The housekeeping gene 18S was used as internal control. Values represent mean±S.D. * P<0.05; ** P<0.01; ***P<0.001 compared to controls.

FIGS. 21A-21B. Mice treated with VNN1 siRNA showed a marked reduction of angiogenesis development during diet-induced NASH. FIG. 21A. QPCR for pro-angiogenic genes VEGF-A and VE-Cadherin mRNA expression in liver samples from C57/B6 mice (n=8) placed on the NASH inducing MCD diet for 6 weeks and treated with VNN1 siRNA, control siRNA and PBS (mock). The housekeeping gene 18S was used as an internal control. FIG. 21B. Representative micrographs of the immunostaining for the neovessel formation marker CD31 in liver sections of MCD-fed mice treated with VNN1 siRNA, control siRNA and PBS (mock). A 20× magnification was used for micrographs. Values represent mean±S.D. * P<0.05; ** P<0.01; ***P<0.001 compared to controls.

FIGS. 22A-22C. Casp3−/− mice are protected from MCD-induced pathological angiogenesis. FIG. 22A. Representative micrographs for H-E staining, immunostaining for CD-31 and vonWillebrand factor (vWF) on liver specimens harvested from WT and Caspase 3 KO mice fed the MCD or control diet (MCS) diet for 6 weeks. A 20× magnification was used for micrographs. FIG. 22B. Total area in pixel (px) is reported in the graph to show the quantification of CD31 immunostaining. FIG. 22C. QPCR of expression of pro-angiogenic VEGF-A and FGF-β mRNA. The housekeeping gene 18S was used as an internal control. Values represent mean±S.D. * P<0.05; ** P<0.01; ***P<0.001 compared to controls.

FIG. 23A-23B. CDAA resembles the histopathological features of human NASH. FIG. 23A. Liver specimens from wild type C57BL/6 mice (n=6) fed with the CDAA (CD), CSAA (CS) or chow diet for 20 weeks, were used for assessing liver damage (Hematossilin & Eosin staining, H&E), cell death (TUNEL staining), fibrosis by detecting the collagen deposition (Sirius red staining) and pathological angiogenesis (CD31 staining). FIG. 23B. Analysis of the expression of the transcripts for VEGF-A, FGF-β, Collagen type-I and α-SMA by quantitative real-time PCR. The housekeeping gene 18S was used as the internal control. Values are mean±standard error. *P<0.05, **P<0.005, ***P<0.0005.

FIGS. 24A-24E. Characterization of blood and liver extracellular vesicles (EV). FIG. 24A. TEM images of blood samples with circulating EVs isolated from CDAA-fed mice for 20 weeks and FIG. 24B hepatic EVs (red arrows) released in the space of Disse (DS). Hepatocytes (H), (Ld) lipid droplet, (m) microvilli. FIG. 24C. Dynamic Light Scattering analysis was performed to measure the size of EVs isolated from the platelet-free plasma of CDAA-fed mice for 20 weeks. The graph shows a predominant peak of big vesicles (mean 530 nm) corresponding to microparticles and a peak of smaller vesicles (mean 50 nm), corresponding to the exosomes. FIG. 24D. Western blot analysis of the circulating exosome (EXO) and microparticles (MP) by using antibodies against vesicles markers (Cd63, Cd81, Icam-1 and Vanin-1). FIG. 24E. Flow cytometry analysis of circulating Calcein+extracellular vesicles (μL of plasma) isolated from CDAA (CD), CSAA (CS) or chow diet fed mice for 20 weeks. *P<0.05, **P<0.005, ***P<0.0005.

FIG. 25. Gene Ontology analysis. Extracellular vesicles were isolated from CDAA-fed mice for 20 weeks and purified by ultracentrifugation. Circulating extracellular vesicle proteins identified by the LC-MS/MS analysis were classified based on molecular function and biological processes according to the GO consortium.

FIGS. 26A-26D. Release of circulating extracellular vesicles is time-dependent and correlates with the histopathological features of NASH. C57BL/6 mice (n=6) were fed with a CDAA (CD) or chow diet for 4 weeks and 20 weeks to mimic an early stage and late stage of NAFLD, respectively. Circulating EVs were stained with 1 μM of Calcein AM and detected by flow cytometry as described in methods. FIG. 26A. FACS analysis detected a massive amount of extracellular vesicles per μL of plasma over time. Levels of circulating extracellular vesicles strongly correlate with the histopathological features of NASH, in particular with (FIG. 26B) cell death, (FIG. 26C) liver fibrosis and (FIG. 26(D)) pathological angiogenesis, as shown by the strongly statistically significant Pearson's coefficients. *P<0.05, **P<0.005, ***P<0.0005.

FIG. 27. CDAA up regulates pathological angiogenesis and fibrosis related genes. Analysis of the expression of transcripts for additional pro-angiogenic (CD126) and pro-fibrogenic (CTGF and TIMP-1) genes in liver samples collected from mice fed a chow, CDAA or CSAA diet for 20 weeks. The housekeeping gene 18S was used as an internal control. Values are mean±standard error. *P<0.05, **P<0.005, ***P<0.0005.

FIGS. 28A-28B. Liver damage, fibrosis, cell death and pathological angiogenesis developed over time in experimental NASH. Wild type C57BL/6 mice (n=6) were fed a CDAA (CD) or chow diet for 4 weeks and 20 weeks to mimic an early stage and late stage of NAFLD, respectively. FIG. 28A. Histopathological representative panels of hematoxilin and eosin (H&E), Sirius red staining to evaluate collagen deposition, TUNEL staining to detect cell death and CD31 to identify neovessels formation. FIG. 28B. Gene expression of VEGF-A, CD126 (VE-Cadherin), FGF-β, α-SMA, CTGF and Collagen type-I was evaluated by quantitative real-time PCR. The housekeeping gene 18S was used as an internal control. Values are mean±standard error. *P<0.05, **P<0.005, ***P<0.0005.

FIGS. 29A-29B illustrate that the number of circulating MPs were increased in mouse models of NAFLD, either wild type fed with a high fat diet or leptin deficient (ob/ob) mouse (FIG. 29A, *** p<0.001); and the number of circulating MPs were decreased when human patients with NAFLD were placed on a low calorie diet for 3 months (FIG. 29B).

FIG. 30 illustrates a change from baseline after 1 year of therapy with Pentoxifylline (PTX) vs. placebo (PLC) in levels of Vanin-1 (pg/mL). In the box-and-whisker plot the lower boundary indicates the 25^{th} percentile, the line within the box indicates the median value and the upper boundary of the box indicates the 75^{th} percentile. The whiskers extend to the most extreme data points.

### DETAILED DESCRIPTION OF THE INVENTION

The present invention may be understood more readily by reference to the following detailed description of the preferred embodiments of the invention and the Examples included herein. However, before the present methods and compositions are disclosed and described, it is to be understood that this invention is not limited to specific methods, specific conditions, specific sequences, specific host cells, etc., as such may, of course, vary, and the numerous modifications and variations therein will be apparent to those skilled in the art. It is also to be understood that the terminology used herein is for the purpose of describing specific embodiments only and is not intended to be limiting. It is also to be understood that as used in the specification and in the claims, “a” or “an” can mean one or more, depending upon the context in which it is used. Thus, for example, reference to “a vesicle” can mean that at least one vesicle can be utilized.

The invention provides a non-invasive or minimally invasive diagnostic method of detecting, monitoring, or assessing the degree, severity, or progression of liver damage in a subject with nonalcoholic fatty liver disease (NAFLD). In contrast to prior art diagnostic methods, the diagnostic method of the invention is able to readily diagnose liver damage using a bodily sample that is obtained from the subject by non-invasive or minimally invasive methods. The bodily sample can include, for example, bodily fluids, such as blood, serum, or plasma that are obtained by minimally invasive methods. The invention can also be used as a diagnostic test to distinguish steatosis from non-alcoholic steatohepatitis (NASH), and detect early stages of liver fibrosis.

The invention also provides a method for monitoring the response of a subject to treatment of liver disease or liver damage and to a method of monitoring the pathogenesis of liver damage caused by an agent administered to a subject. The invention may also be used to detect or monitor the progression of other forms of liver disease, besides NAFLD, such as Alagille syndrome, α-1-antitrypsin deficiency, autoimmune hepatitis, biliary atresia, chronic hepatitis, cancer of the liver, cirrhosis, liver cysts, fatty liver, galactosemia, Gilbert's syndrome, primary biliary cirrhosis, hepatitis A, hepatitis B, hepatitis C, primary sclerosing cholangitis, Reye's syndrome, sarcoidosis, tyrosinemia, type I glycogen storage disease, Wilson's disease, hemochromatosis, and neonatal hepatitis.

Unless specifically addressed herein, all terms used have the same meaning as would be understood by those of skilled in the art of the present invention. The following definitions will provide clarity with respect to the terms used in the specification and claims to describe the present invention.

The term “monitoring” as used herein refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

The term “quantitative data” as used herein refers to data associated with any dataset components (e.g., markers, clinical indicia, metabolic measures, or genetic assays) that can be assigned a numerical value. Quantitative data can be a measure of the level of a marker and expressed in units of measurement, such as molar concentration, concentration by weight, etc. For example, if the marker is the circulating extracellular vesicles (EVs) or hepatocyte-derived microparticles (MPs), or biomarkers expressed thereon, quantitative data for that marker can be the EVs or MPs or the biomarkers measured using methods known to those skilled in the art and expressed in mM or mg/dL concentration units.

The term “subject” as used herein relates to an animal, such as a mammal including a small mammal (e.g., mouse, rat, rabbit, or cat) or a larger mammal (e.g., dog, pig, or human). In particular aspects, the subject is a large mammal, such as a human, that is suspected of having or at risk of NAFLD, NASH, or other liver damage or diseases.

The term “diagnosing NAFLD, NASH, or other liver damage or diseases” as used herein refers to a process aimed at one or more of: determining if a subject is afflicted with NAFLD, NASH, or other liver damage or diseases; determining the severity or stage of NAFLD, NASH, or other liver damage or disease related pathologies in a subject; determining the risk that a subject is afflicted with NAFLD, NASH, or other liver damage or diseases; and determining the prognosis of a subject afflicted with NAFLD, NASH, or other liver damage or diseases.

The terms “biological sample” or “bodily sample” are used herein in its broadest sense. A biological or bodily sample may be obtained from a subject (e.g., a human) or from components (e.g., tissues) of a subject. The sample can be obtained either invasively or non-invasively from the subject but is preferably obtained non-invasively. The sample includes any biological sample that is suspected of containing EVs, MPs, and/or any biomarkers of interest. The sample obtained from the subject can potentially include body fluids, such as blood, plasma, serum, urine, blood, fecal matter, saliva, mucous, and cell extract as well as solid tissue, such as cells, a tissue sample, or a tissue or fine needle biopsy samples; and archival samples with known diagnosis, treatment and/or outcome history. Frequently, the sample will be a “clinical sample”, i.e., a sample derived from a patient. The sample also encompasses any material derived by processing the biological sample. Processing of the sample may involve one or more of, filtration, distillation, extraction, concentration, inactivation of interfering components, addition of reagents, and the like. It will be appreciated by one skilled in the art that other biological or bodily samples not listed can also be used in accordance with the present invention.

The terms “normal” and “healthy” are used herein interchangeably. They refer to an individual or group of control individuals who have not shown any symptoms of NAFLD, NASH, or other liver damage or diseases, such as liver inflammation, fibrosis, steatosis, and have not been diagnosed with NAFLD, NASH, or other liver damage or diseases. The normal individual (or group of individuals) is not on medication affecting NAFLD, NASH, or other liver damage or diseases. In certain embodiments, normal individuals have similar sex, age, body mass index as compared with the individual from which the sample to be tested was obtained. The term “normal” is also used herein to qualify a sample isolated from a healthy individual.

The subject may be an “apparently healthy” subject. “Apparently healthy”, as used herein, means individuals who have not been previously diagnosed with liver damage, liver disease and/or who have not been previously diagnosed as having any signs or symptoms indicating the presence of liver damage or liver disease. Additionally, apparently healthy subjects may include those individuals having low or no risk for developing liver disease. In addition to apparently healthy subjects, subjects may include individuals having pre-existing liver disease and/or may be at an elevated risk of developing liver damage or liver disease. Subjects having an elevated risk of developing liver damage or liver disease can include, for example, individuals with a family history of liver disease, elevated serum alanine aminotransferase (ALT) and glutamyl-transferase (GGT) activity, hepatitis B surface antigen, hepatitis C virus-RNA positivity, visceral obesity, elevated lipid levels, insulin resistance, hyperglycemia, and hypertension. Subjects at risk of having or developing liver disease, e.g., NAFLD, can also include individuals with elevated liver enzymes and evidence of clinical components of the metabolic syndrome (e.g., any one of obesity, diabetes, hypertension, and hyperlipidemia) in the absence of alternate causes of elevated ALTs.

The terms “control” or “control sample” or “control dataset” as used herein refer to one or more biological samples isolated from an individual or group of individuals that are normal (i.e., healthy). The term “control”, “control value” or “control sample” can also refer to the compilation of data derived from samples of one or more individuals classified as normal, one or more individuals diagnosed with NAFLD, one or more individuals diagnosed with NASH, one or more individuals diagnosed with hepatic steatosis, and/or one or more individuals diagnosed with liver fibrosis.

The term “indicative of NAFLD, NASH, or other liver damage or diseases” as used herein, when applied to an amount of circulating extracellular vesicles (EVs) or hepatocyte-derived microparticles (MPs) or at least one biomarker expressed in the EVs or MPs in a sample, refers to a level or an amount, which is diagnostic of NAFLD, NASH, or other liver damage or diseases such that the level is found significantly more often in subjects with the disease than in patients without the disease or another stage of NAFLD, such as hepatic steatosis (as determined using routine statistical methods setting confidence levels at a minimum of 95%). In certain embodiments, a level, which is indicative of NAFLD, NASH, or other liver damage or diseases, is found in at least about 60%, at least about 70%, at least about 75%, at least about 80%, at least about 85%, at least about 90%, at least about 95% or more in patients who have the disease of patients and is found in less than about 10%, less than about 8%, less than about 5%, less than about 2.5%, or less than about 1% of subjects who do not have the disease.

The term “biomarker” as used herein refers to an indicator and/or prognostic factor of biologic or pathologic processes or pharmacologic responses to a therapeutic intervention. As used herein, the term “prognostic factor” refers to any molecules and/or substances contributing to a predicted and/or expected course of NAFLD, NASH, angiogenesis associated with NASH, liver fibrosis, or other liver damage or diseases in a subject including various developments, changes and outcomes of the disease. As used herein, the term “detecting reagents” refer to any substances, chemicals, solutions used in chemical reactions and processes that are capable of detecting, measuring, and examining biomarker of interest, and isoforms thereof. In certain preferred embodiments, the biomarker refers to the circulating extracellular vesicles (EVs) or hepatocyte-derived microparticles (MPs) detected and/or associated with NAFLD, NASH, liver fibrosis and their associated liver damage. In other embodiments, the biomarker refers to the gene or protein molecules expressed or detected in the EVs or MPs. The protein biomarkers expressed and/or detected in the EVs or MPs include, but are not limited to, those listed in Tables 1-4 below. In other preferred embodiments, the biomarker detecting reagents used herein comprise chemicals, substances, and solutions that are suitable for determining either mRNA or protein, or both expression levels of the biomarker of interest, or isoforms or associated molecules thereof.

As used herein, the term isoforms or homologs of a biomarker of interest refer to a protein, or its encoded nucleic acid, having at least 60%, 75%, 85%, 90%, 95%, 96%, 97%, 98%, 99% or more identical, to a wild type of the protein biomarker core amino acid domain, or the nucleic acid domain encoding the said core amino acid domain. Identity can be determined using the BLAST program on default settings. As used herein, the core domain comprises one or more biologically active portions of the proteins or the nucleic acid portions encoding said proteins. As used herein, the “biologically active portions” include one or more fragments of the protein, or the nucleic acid fragment encoding said protein, comprising amino acid or nucleic acid sequences sufficiently homologous to, or derived from, the amino acid or nucleic acid sequence of the proteins, or their nucleic acids, which include fewer amino acids, or nucleic acids than the full length protein or its nucleic acid, and exhibit at least one activity of the full-length protein. Typically a biologically active portion comprises a domain or motif with at least one activity of the protein. A biologically active portion of a protein can be a polypeptide which is, for example, 10, 25, 50, 100, 200 or more amino acids in length. In one embodiment, a biologically active portion of these proteins can be used as a target for developing agents which modulate activities of these proteins.

Moreover, the protein biomarkers used herein include the proteins and/or enzymes encoded by polynucleotides that hybridize to the polynucleotide encoding these proteins under stringent conditions. As used herein, “hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under different stringent conditions. The invention includes polynucleotides capable of hybridizing under reduced stringency conditions, certain stringent conditions, or highly stringent conditions, to polynucleotides encoding the protein biomarker of interest described herein. As used herein, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in certain embodiments, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al., eds., Greene Publishing and Wiley-Interscience, New York, 1995; and Tijssen, 1993, Laboratory Techniques in Biochemistry and Molecular Biology: Hybridization with Nucleic Acid Probes, Part I, Chapter 2, Elsevier, New York, 1993.

As used herein, the term “expression level” refers to an amount of a gene and/or protein that is expressed in a cell. As used herein, a “gene” includes a polynucleotide containing at least one open reading frame that is capable of encoding a particular polypeptide or protein after being transcribed and translated. Any of the polynucleotide sequences described herein may also be used to identify larger fragments or full-length coding sequences of the gene with which they are associated. Methods of isolating larger fragment sequences are known to those of skill in the art.

As used herein, the term “protein” or “polypeptide” is interchangeable, and includes a compound of two or more subunit amino acids, amino acid analogs, or peptidomimetics. The subunits may be linked by peptide bonds. In another embodiment, the subunit may be linked by other bonds, e.g., ester, ether, etc. As used herein, the term “amino acid” includes either natural and/or unnatural or synthetic amino acids, including both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

As used herein, the terms “polynucleotide,” “nucleic acid/nucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, DNA, cDNA, genomic DNA, recombinant polynucleotides, branched polynucleotides, plasmids, vectors, isolated DNA of any sequence, isolated RNA of any sequence, nucleic acid probes, and primers. Polynucleotides may be naturally-occurring, synthetic, recombinant or any combination thereof.

As used herein, a “naturally-occurring” polynucleotide molecule includes, for example, an RNA (mRNA) or DNA molecule having a nucleotide sequence that occurs in nature (e.g., encodes a natural protein). As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant” also encompasses the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence amplified using a primer of the invention.

A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form. The “polynucleotide sequence” is the alphabetical representation of a polynucleotide molecule. A polynucleotide is composed of a specific sequence of four nucleotide bases: adenine (A); cytosine (C); guanine (G); thymine (T); and uracil (U) in place of guanine when the polynucleotide is RNA. This alphabetical representation can be inputted into databases in a computer and used for bioinformatics applications such as, for example, functional genomics and homology searching.

As used herein, the term “primer” refers to a segment of DNA or RNA that is complementary to a given DNA or RNA sequences (e.g. sequences of a particular biomarker of interest or its isoform) and that is needed to initiate replication by DNA polymerase, and a term “probe” refers to a substance, such as DNA, that is radioactively labeled or otherwise marked and used to detect or identify another substance in a sample. As used herein, the term “primer” and “probe” are used interchangeably, and typically comprise a substantially isolated oligonucleotide typically comprising a region of nucleotide sequence that hybridizes under stringent conditions to at least about 12, preferably about 25, more preferably about 40, 50, or 75 consecutive nucleotides of a sense and/or an antisense strands of a nucleotide sequence of a biomarker of interest and its isoforms thereof; or naturally occurring mutants thereof. As used herein, primers based on the nucleotide sequence of a biomarker of interest, and isoforms thereof, can be used in PCR reactions to clone homologs of the biomarker and its isoforms. Probes based on the nucleotide sequences of the biomarker of interest, and isoforms thereof, can be used to detect transcripts or genomic sequences encoding the same or substantially identical polypeptides or proteins. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which express or over-express the biomarker of interest, or isoforms thereof, such as by measuring a level of encoding nucleic acid, in a sample of cells, e.g., detecting mRNA levels or determining whether a genomic gene has been mutated or deleted.

As used herein, the term “therapeutic agent” refers to any molecules naturally occurred or synthesized, including but not limited to, small molecule, biologics, peptide, proteins, or antibodies. The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target protein biomarker and its isoforms of interest. The term “antibody fragments” comprise a portion of a full length antibody, generally the antigen binding or variable region thereof. Examples of antibody fragments include Fab, Fab′, F(ab′)², and Fv fragments. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

The term “single-chain Fv” or “sFv” antibody fragments comprise the VH and VL domains of antibody, wherein these domains are present in a single polypeptide chain. Generally, the Fv polypeptide further comprises a polypeptide linker between the VH and VL domains which enables the sFv to form the desired structure for antigen binding.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-polyacrylamide gel electrophoresis under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

In order to avoid potential immunogenicity of the monoclonal antibodies in humans, the monoclonal antibodies that have the desired function are preferably human or humanized. “Humanized” forms of non-human (e.g., murine) antibodies are chimeric antibodies that contain minimal sequence derived from non-human immunoglobulin. For the most part, humanized antibodies are human immunoglobulins (recipient antibody) in which hyper variable region residues of the recipient are replaced by hyper variable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hyper variable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. Antibodies capable of immunoreacting to particular protein biomarker of interest and their isoforms are made using conventional methods known in the art.

The therapeutic agent may also refer to any oligonucleotides (antisense oligonucleotide agents), polynucleotides (e.g. therapeutic DNA), ribozymes, dsRNAs, siRNA, RNAi, and/or gene therapy vectors. The term “antisense oligonucleotide agent” refers to short synthetic segments of DNA or RNA, usually referred to as oligonucleotides, which are designed to be complementary to a sequence of a specific mRNA to inhibit the translation of the targeted mRNA by binding to a unique sequence segment on the mRNA. Antisense oligonucleotides are often developed and used in the antisense technology. The term “antisense technology” refers to a drug-discovery and development technique that involves design and use of synthetic oligonucleotides complementary to a target mRNA to inhibit production of specific disease-causing proteins. Antisense technology permits design of drugs, called antisense oligonucleotides, which intervene at the genetic level and inhibit the production of disease-associated proteins. Antisense oligonucleotide agents are developed based on genetic information.

As an alternative to antisense oligonucleotide agents, ribozymes or double stranded RNA (dsRNA), RNA interference (RNAi), and/or small interfering RNA (siRNA), can also be used as therapeutic agents for regulation of gene expression in cells. As used herein, the term “ribozyme” refers to a catalytic RNA-based enzyme with ribonuclease activity that is capable of cleaving a single-stranded nucleic acid, such as an mRNA, to which it has a complementary region. Ribozymes can be used to catalytically cleave target mRNA transcripts to thereby inhibit translation of target mRNA. The term “dsRNA,” as used herein, refers to RNA hybrids comprising two strands of RNA. The dsRNAs can be linear or circular in structure. The dsRNA may comprise ribonucleotides, ribonucleotide analogs, such as 2′-O-methyl ribosyl residues, or combinations thereof. The term “RNAi” refers to RNA interference or post-transcriptional gene silencing (PTGS). The term “siRNA” refers to small dsRNA molecules (e.g., 21-23 nucleotides) that are the mediators of the RNAi effects. RNAi is induced by the introduction of long dsRNA (up to 1-2 kb) produced by in vitro transcription, and has been successfully used to reduce gene expression in variety of organisms. In mammalian cells, RNAi uses siRNA (e.g. 22 nucleotides long) to bind to the RNA-induced silencing complex (RISC), which then binds to any matching mRNA sequence to degrade target mRNA, thus, silences the gene.

As used herein, the therapeutic agents may also include any vectors/virus used for gene therapy. The term “gene therapy” refers to a technique for correcting defective genes or inhibiting or enhancing genes responsible for disease development. Such techniques may include inserting a normal gene into a nonspecific location within the genome to replace a nonfunctional gene; swapping an abnormal gene for a normal gene through homologous recombinants, repairing an abnormal gene to resume its normal function through selective reverse mutation; and altering or regulating gene expression and/or functions of a particular gene. As used herein, a term “vector/virus” refers to a carrier molecule that carries and delivers the “normal” therapeutic gene to the patient's target cells. Because viruses have evolved a way of encapsulating and delivering their genes to human cells in a pathogenic manner, most common vectors for gene therapy are viruses that have been genetically altered to carry the normal human DNA. As used herein, the viruses/vectors for gene therapy include retroviruses, adenoviruses, adeno-associated viruses, and herpes simplex viruses. The term “retrovirus” refers to a class of viruses that can create double-stranded DNA copies of their RNA genomes, which can be further integrated into the chromosomes of host cells, for example, Human immunodeficiency virus (HIV) is a retrovirus. The term “adenovirus” refers to a class of viruses with double-stranded DNA genomes that cause respiratory, intestinal, and eye infections in humans, for instance, the virus that cause the common cold is an adenovirus. The term “adeno-associated virus” refers to a class of small, single-stranded DNA viruses that can insert their genetic material at a specific site on chromosome 19. The term “herpes simplex viruses” refers to a class of double-stranded DNA viruses that infect a particular cell type, neurons. Herpes simplex virus type 1 is a common human pathogen that causes cold sores.

As used herein, the term “biologically effective amount” or “therapeutically effective amount” of therapeutic agent is intended to mean a nontoxic but sufficient amount of such therapeutic agents to provide the desired therapeutic effect. The amount that is effective will vary from subject to subject, depending on the age and general condition of the individual, the particular active agent or agents, and the like. Thus, it is not always possible to specify an exact effective amount. However, an appropriate effective amount in any individual case may be determined by one of ordinary skill in the art using routine experimentation.

As used herein, the term “pharmaceutical composition” contemplates compositions comprising one or more therapeutic agents as described above, and one or more pharmaceutically acceptable excipients, carriers, or vehicles. As used herein, the term “pharmaceutically acceptable excipients, carriers, or vehicles” comprises any acceptable materials, and/or any one or more additives known in the art. As used herein, the term “excipients,” “carriers” or “vehicle” refer to materials suitable for drug administration through various conventional administration routes known in the art. Excipients, carriers, and vehicles useful herein include any such materials known in the art, which are nontoxic and do not interact with other components of the composition in a deleterious manner.

One aspect of the invention therefore relates to a method of predicting, detecting, monitoring, or assessing the degree or severity of NAFLD, NASH, angiogenesis associated with NASH, or other liver damage or diseases in a subject. In certain embodiments, the method includes obtaining a bodily sample from the subject, and determining an amount of circulating EVs in the sample. An increased level of the circulating EVs in the subject compared to a control is indicative of an increase in degree or severity of NAFLD and potentially nonalcoholic steatohepatitis (NASH), angiogenesis or liver fibrosis or other damage in the subject. In certain embodiments, the majority of the circulating EVs are hepatocyte-derived microparticles (MPs). In these embodiments, an increased level of the circulating MPs in the subject compared to a control is indicative of an increase in degree or severity of NAFLD and potentially nonalcoholic steatohepatitis (NASH), angiogenesis or liver fibrosis or other damage in the subject.

In other embodiments, the invention method comprises detecting and determining an expression level of at least one biomarker expressed or detected in the circulating EVs and/or MPs in a bodily sample. These biomarkers are involved in molecule function and cellular localization. In certain embodiments, the biomarkers expressed or detected in the circulating EVs or MPs are involved in capsase 3 activation. Exemplary biomarkers expressed in the circulating EVs or MPs are listed in Tables 1-4 below (See EXAMPLES). In one embodiment, the biomarker is Vanin-1 protein.

In certain embodiments, the bodily sample can comprise a blood sample obtained non-invasively from the subject. In some aspects, the amount of blood taken from a subject is about 0.1 ml or more. In an exemplary embodiment, the bodily sample is blood plasma isolated from a whole blood sample obtained from a subject. Blood plasma may be isolated from whole blood using well known methods, such as centrifugation. The bodily samples can be obtained from the subject using sampling devices, such as syringes, swabs or other sampling devices used to obtain liquid and/or solid bodily samples either invasively (i.e., directly from the subject) or non-invasively. These samples can then be stored in storage containers. The storage containers used to contain the collected sample can comprise a non-surface reactive material, such as polypropylene. The storage containers should generally not be made from untreated glass or other sample reactive material to prevent the sample from becoming absorbed or adsorbed by surfaces of the glass container.

Collected samples stored in the container may be stored under refrigeration temperature. For longer storage times, the collected sample can be frozen to retard decomposition and facilitate storage. For example, samples obtained from the subject can be stored in a falcon tube and cooled to a temperature of about −80° C. The collected bodily sample can be stored in the presence of a chelating agent, such as ethylenediaminetetraacetic acid (EDTA). The collected bodily sample can also be stored in the presence of an antioxidant, such as butylated hydroxytoluene (BHT) or diethylenetriamine pentaacetic acid, and/or kept in an inert atmosphere (e.g., overlaid with argon) to inhibit oxidation of the sample.

Bodily samples obtained from the subject can then be contacted with a solvent, such as an organic solvent. The solvent can include any chemical useful for the removal (i.e., extraction) of the EVs and/or MPs of interest from a bodily sample. For example, where the bodily sample comprises plasma, the solvent can include a water/methanol mixture. It will be appreciated by one skilled in the art that the solvent is not strictly limited to this context, as the solvent may be used for the removal of lipids from a liquid mixture, with which the liquid is immiscible in the solvent. Those skilled in the art will further understand and appreciate other appropriate solvents that can be employed to extract lipids from the bodily sample. The solvent can include solvent mixtures comprising miscible, partially miscible, and/or immiscible solvents. The solvent can also be combined with other solvents which can act as carriers facilitating mixing of the solvent with the bodily sample or transfer of the extracted EVs and/or MPs from the bodily sample.

The bodily sample may be pre-treated as necessary by dilution in an appropriate buffer solution, heparinized, concentrated if desired, or fractionated by any number of methods including, but not limited to, ultracentrifugation, fractionation by fast performance liquid chromatography, or other known methods. Any of a number of standard aqueous buffer solutions, employing one of a variety of buffers, such as phosphate, Tris, or the like at a physiological pH can be used.

After obtaining the bodily sample (e.g., blood, serum, plasma), the amount of EVs and/or MPs in the bodily sample, or an expression level of one or more biomarkers expressed in the EVs or MPs, is detected, measured, and/or quantifying to determine the level of EVs, MPs, or the biomarkers of interest in the subject. An increase in the amount of EVs or MPs, or the expression level of the biomarker of interest is associated with an increase in liver damage and/or liver disease.

In certain embodiments, the circulating EVs or MPs in the bodily sample, as well as protein biomarkers expressed therein, can be detected and/or quantified using an immunoassay, such as an enzyme-linked immunoabsorbent assay (ELISA), or other assays, now known or later developed, that can be used to detect and/or quantify EVs, MPs, and biomarkers of interest in the bodily sample. These assays include, but are not limited to, flow cytometry (FACS) analysis, radioimmunoassays, both solid and liquid phase, fluorescence-linked assays, competitive immunoassays, mass spectrometry (MS)-based methods (e.g., liquid chromatography MS), and HPLC.

Once the level or amount of the EVs or MPs or the expression level of the biomarker of interest in a sample are determined, the level/amount can be compared to a predetermined value or control value to provide information for diagnosing, monitoring, or assessing NAFLD, NASH, and/or liver fibrosis in a subject. For example, the level/amount of EVs, MPs or the expression level of the biomarker expressed therein in a sample can be compared to a predetermined value or control value to determine if a subject is afflicted with NAFLD, NASH, liver fibrosis, or other liver damage or diseases.

The level/amount of EVs, MPs, or the expression level of the biomarkers expressed therein, in the subject's bodily sample may also be compared to the level/amount of the EVs or MPs, or the expression level of the biomarkers of interest obtained from a bodily sample previously obtained from the subject, such as prior to administration of therapeutic. Accordingly, the method described herein can be used to measure the efficacy of a therapeutic regimen for the treatment of NAFLD, NASH, liver fibrosis, or other liver damage or diseases in a subject by comparing the level/amount of EVs, MPs, or the expression level of the biomarkers of interest in bodily samples obtained before and after a therapeutic regimen. Additionally, the method described herein can be used to measure the progression of NAFLD, NASH, liver fibrosis, or other liver damage or diseases in a subject by comparing the level/amount of EVs, MPs, or the expression level of the biomarker of interest in a bodily sample obtained over a given time period, such as days, weeks, months, or years.

The level/amount of EVs, MPs, or the expression level of the biomarker of interest in a sample may also be compared to a predetermined value or control value to provide information for determining the severity of the disease in the subject or the tissue of the subject (e.g., liver tissue). Thus, in some aspect, a level/amount of EVs, MPs, or the expression level of the biomarker of interest may be compared to control values obtained from subjects with well-known clinical categorizations, or stages, of histopathologies related to NAFLD and/or NASH (e.g., lobular liver inflammation, liver steatosis, and liver fibrosis). In one particular embodiment, a level/amount of EVs, MPs, or the expression level of the biomarker of interest in a sample can provide information for determining a particular stage of fibrosis in the subject. For example, stages of fibrosis may be defined as Stage 1: no fibrosis or mild fibrosis; Stage 2: moderate fibrosis; Stage 3 and 4: severe fibrosis.

A predetermined value or control value can be based upon the level/amount of EVs, MPs, or the expression level of the biomarker of interest in comparable samples obtained from a healthy or normal subject or the general population or from a select population of control subjects. In some aspects, the select population of control subjects can include individuals diagnosed with NAFLD and/or NASH. For example, a subject having a greater level/amount of EVs, MPs, or the expression level of the biomarker of interest compared to a control value may be indicative of the subject having a more advanced stage of a histopathology related to NASH.

The select population of control subjects may also include subjects afflicted with NALFD in order to distinguish subjects afflicted with NASH from those with hepatic steatosis by comparing the level/amount of EVs, MPs, or the expression level of the biomarker of interest in the samples. In some aspects, the select population of control subjects includes individuals afflicted with NALFD having none or minimal steatosis and none or minimal inflammation and who were classified as normal liver biopsy. In some aspects, the select population of control subjects may include a group of individuals afflicted with hepatic steatosis. In another aspect, the select population of control subjects can include individual patients with chronic hepatitis C or alcohol liver disease in order to distinguish subjects afflicted with NASH from those with other chronic liver diseases by comparing the level/amount of EVs, MPs, or the expression level of the biomarker of interest in the samples.

The predetermined value can be related to the value used to characterize the level/amount of EVs, MPs, or the expression level of the biomarker of interest in the bodily sample obtained from the test subject. Thus, if the level/amount of EVs, MPs, or the expression level of the biomarker of interest is an absolute value, the predetermined value can also be based upon the absolute value in subjects in the general population or a select population of human subjects. Similarly, if the level/amount of EVs, MPs, or the expression level of the biomarker of interest is a representative value such as an arbitrary unit, the predetermined value can also be based on the representative value.

The predetermined value can take a variety of forms. The predetermined value can be a single cut-off value, such as a median or mean. The predetermined value can be established based upon comparative groups such as where the level/amount of EVs, MPs, or the expression level of the biomarker of interest in one defined group is double the level/amount of EVs, MPs, or the expression level of the biomarker of interest in another defined group. The predetermined value can be a range, for example, where the general subject population is divided equally (or unequally) into groups, or into quadrants, the lowest quadrant being subjects with the lowest level/amount of EVs, MPs, or the expression level of the biomarker of interest, the highest quadrant being individuals with the highest level/amount of EVs, MPs, or the expression level of the biomarker of interest. In an exemplary embodiment, two cutoff values are selected to minimize the rate of false positive and negative results.

Predetermined values of the EVs, MPs or the expression level of the biomarkers expressed therein, such as for example, mean levels, median levels, or “cut-off” levels, are established by assaying a large sample of subjects in the general population or the select population and using a statistical model such as the predictive value method for selecting a positively criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for EVs, MPs, or the expression level of each biomarker that is assayed.

In other embodiments, the invention relates to a method for generating a result useful in diagnosing and monitoring NAFLD, NASH, liver fibrosis, or other liver damage or diseases by obtaining a dataset associated with a sample, where the dataset includes quantitative data about the amounts of EVs, MPs, or the expression level of the biomarkers expressed therein which have been found to be predictive of severity of NASH and/or liver fibrosis with a statistical significance less than 0.2 (e.g., p value less than about 0.05), and inputting the dataset into an analytical process that uses the dataset to generate a result useful in diagnosing and monitoring NAFLD, NASH, liver fibrosis, or other liver damage or diseases. In certain embodiments, the dataset also includes quantitative data about other clinical indicia or other marker associated with NAFLD.

Datasets containing quantitative data, typically the level/amount of EVs, MPs, or the expression levels of the biomarker of interest used herein, and quantitative data for other dataset components can be inputted into an analytical process and used to generate a result. The analytical process may be any type of learning algorithm with defined parameters, or in other words, a predictive model. Predictive models can be developed for a variety of NAFLD classifications by applying learning algorithms to the appropriate type of reference or control data. Multivariable modeling can be applied to generate a risk score for diagnosing NASH. A risk score can be derived from the amount of total- or hepatocyte-derived EVs or MPs or the expression level of the biomarker of interest as determined by the methods described herein. The risk score can be compared to a control value, to provide information for diagnosing NASH in a subject. The result of the analytical process/predictive model can be used by an appropriate individual to take the appropriate course of action.

In certain embodiments, a scoring system or risk score can be generated by the analytical process to diagnose and monitor NAFLD, NASH, liver fibrosis, or other liver damage and diseases. In some aspects, the analytical process can use a dataset that includes the level/amount of total- or hepatocyte-derived EVs, MPs, or the expression level of the biomarker of interest in a subject's sample as determined by the methods described herein. The risk score can then be compared to a control value, to provide information for diagnosing or monitoring or assessing NASH and/or liver fibrosis or other liver damage or diseases in a subject.

In other aspects, the analytical process can use a reference dataset that includes the determined level/amount of EVs, MPs, or the expression level of the biomarker of interest and quantitative data from one or more clinical indicia to generate a risk score. The risk score can be derived using an algorithm that weights the level/amount of EVs, MPs, or the expression level of the biomarker of interest in the sample and one more clinical indicia (or anthropometric features or measures) including but not limited to, age, gender, race, with or without diabetes, with or without hypertension, with or without hyperlipidemia, BMI, weight, height, waist circumference, hip/waste ratio, and other laboratory data including but not limited to aspartate aminotransferase (AST), alanine aminotransferase (ALT), AST/ALT ratio, gamma GT, bilirubin, alkaline phosphatase, albumin, prothrombin time, platelet count, creatinine, total cholesterol, HDL, LDL, Triglycerides, triglyceride:HDL ratio, fasting glucose, fasting insulin, glucose/insulin ratio and Homeostatic Model Assessment index measuring insulin resistance. By way of example, a score derived from the formula: risk score=−10.051+0.0463*Age (year)+0.147*BMI (kg/m²)+0.0293*AST (IU/L)+2.658*Total EVs (EV number/microliter).

In certain embodiments, the one or more clinical indicia can include at least one of the subject's age, body mass index, or concentration of aspartate transaminase or alanine transaminase. In other embodiments, the one or more clinical indicia can include at least two of the subject's age, body mass index, and concentration of aspartate transaminase or alanine transaminase. In other embodiments, the dataset can include the determined level/amount of EVs, MPs, or the expression level of the biomarker of interest, the subject's age, body mass index, and concentration of aspartate transaminase or alanine transaminase.

The analytical process used to generate a risk score may be any type of process capable of providing a result useful for classifying a sample, for example, comparison of the obtained dataset with a reference dataset, a linear algorithm, a quadratic algorithm, a decision tree algorithm, or a voting algorithm. Prior to input into the analytical process, the data in each dataset can be collected by measuring the values for EVs, MPs, or the biomarkers expressed therein usually in triplicate or in multiple triplicates. The data may be manipulated, for example, raw data may be transformed using standard curves, and the average of triplicate measurements used to calculate the average and standard deviation for each patient. These values may be transformed before being used in the models, e.g. log-transformed or Box-Cox transformed. This data can then be input into the analytical process with defined parameters. The analytical process may set a threshold for determining the probability that a sample belongs to a given class. The probability preferably is at least 50%, at least 60%, at least 70%, at least 80%, at least 90%, or higher.

In certain embodiments, the analytical process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method. In some embodiments, the analytical process is based on a regression model, preferably a logistic regression model. Such a regression model includes a coefficient for EVs, MPs, or each of the biomarkers in a selected set of biomarkers disclosed herein. In such embodiments, the coefficients for the regression model are computed using, for example, a maximum likelihood approach. In particular embodiments, molecular marker data from the two groups (e.g., healthy and diseased) is used and the dependent variable is the status of the patient for which marker characteristic data are from.

By way of example, the analytical process can include a logistic regression model that generates a risk score based on the following algorithm: risk score=[−10.051+0.0463*Age(years)+0.147*BMI(kg/m²)+0.0293*AST(IU/L)+2.658*Total EVs (EV number/microliter)]*10) is determined. The risk score can be converted to a probability distribution with a value of 0 to 100 by the following algorithm; evNASH=100*exp(z)/[1+exp(z)], wherein evNASH is the probability distribution and z is the risk score calculated using the above noted algorithm.

It will be appreciated, that other analytical processes can be used to generate a risk score. These analytical processes can include for example a Linear Discriminant Analysis model, a support vector machine classification algorithm, a recursive feature elimination model, a prediction analysis of microarray model, a classification and regression tree (CART) algorithm, a FlexTree algorithm, a random forest algorithm, a multiple additive regression tree (MART) algorithm, or Machine Learning algorithms.

A risk score or result generated by the analytical process can be any type of information useful for making a NAFLD classification, e.g., a classification, a continuous variable, or a vector. For example, the value of a continuous variable or vector may be used to determine the likelihood that a sample is associated with a particular classification.

NAFLD classification refer to any type of information or the generation of any type of information associated with NAFLD, NASH, and/or liver fibrosis, for example, diagnosis, staging, assessing extent of NAFLD, NASH, and/or liver fibrosis progression, prognosis, monitoring, therapeutic response to treatments, screening to identify compounds that act via similar mechanisms as known NAFLD, NASH, and/or liver fibrosis treatments.

In some aspects, the result is used for diagnosis or detection of the occurrence of NASH. In this embodiment, a reference or training set containing “healthy” and “NASH” samples is used to develop a predictive model. A dataset, preferably containing level/amount of EVs, MPs, or the expression level of the biomarker expressed therein, indicative of NASH, is then inputted into the predictive model in order to generate a result. The result may classify the sample as either “healthy” or “NASH” or staging of “NASH”. In other embodiments, the result is a continuous variable providing information useful for classifying the sample, e.g., where a high value indicates a high probability of being a “NASH” sample and a low value indicates a low probability of being a “healthy” sample.

In other embodiments, the result is used for NAFLD, NASH, and/or liver fibrosis staging. In these embodiments, a reference or training dataset containing samples from individuals with disease at different stages is used to develop a predictive model. The model may be a simple comparison of an individual dataset against one or more datasets obtained from disease samples of known stage or a more complex multivariate classification model. In certain embodiments, inputting a dataset into the model will generate a result classifying the sample from which the dataset is generated as being at a specified NAFLD, NASH, and/or liver fibrosis disease stage. Similar methods may be used to provide NAFLD, NASH, and/or liver fibrosis prognosis, except that the reference or training set will include data obtained from individuals who develop disease and those who fail to develop disease at a later time.

In other embodiments, the result is used determine response to NAFLD, NASH, and/or liver fibrosis treatments. In this embodiment, the reference or training dataset and the predictive model is the same as that used to diagnose NAFLD, NASH, and/or liver fibrosis (samples of from individuals with disease and those without). However, instead of inputting a dataset composed of samples from individuals with an unknown diagnosis, the dataset is composed of individuals with known disease which have been administered a particular treatment and it is determined whether the samples trend toward or lie within a normal, healthy classification versus an NAFLD, NASH, and/or liver fibrosis classification.

In other embodiments, the result is used for drug screening, i.e., identifying new agents that target EVs, MPs, or the biomarkers expressed therein to either internalize the circulating EVs or MPs in to the endothelial cells, or reduce the expression level of the biomarkers expressed therein so as to inhibit liver damage or hepatocyte lipotoxicity to angiogenesis and disease progression. Any drug screening methods now known or later developed in the art will be encompassed by the invention. In certain embodiments, the invention provides a drug screening for an agent that is capable of internalizing circulating EVs or MPs into the endothelial cells. In other embodiments, the invention provides a drug screening for an agent that is capable of interacting with at least one biomarker listed in Tables 1-4, which are expressed in the circulating EVs or MPs. In other embodiments, the invention provides a drug screening for an agent that is capable of inhibiting caspase 3 activation.

In one embodiment, the biomarker is Vanin-1, and the agent is capable of blocking Vanin-1 gene or protein expression and/or activities associated with, such agent includes, but not limited to, siRNA and/or antisense against Vanin-1 gene, anti-Vanin-1 antibody, or synthetic small molecule that interacts with Vanin-1 gene or protein so as to inhibit its expression and/or activity level.

In other embodiments, the result is used for drug screening, i.e., identifying compounds that act via similar mechanisms as known NAFLD, NASH, and/or liver fibrosis drug treatments. In this embodiment, a reference or training set containing individuals treated with a known NAFLD, NASH, and/or liver fibrosis drug treatment and those not treated with the particular treatment can be used develop a predictive model. A dataset from individuals treated with a compound with an unknown mechanism is input into the model. If the result indicates that the sample can be classified as coming from a subject dosed with a known NAFLD, NASH, and/or liver fibrosis drug treatment, then the new compound is likely to act via the same mechanism.

One of skill will also recognize that the results generated using these methods can be used in conjunction with any number of the various other methods known to those of skill in the art for diagnosing and monitoring NAFLD, NASH, liver fibrosis, or other liver damage or diseases.

Using methods described herein, skilled physicians may select and prescribe treatments adapted to each individual subject based on the diagnosis of NAFLD, NASH, and/or liver fibrosis provided to the subject through determination of the level/amount of EVs, MPs, or the expression level of the biomarkers expressed therein in a subject's sample. In particular, the present invention provides physicians with a non-subjective means to diagnose NAFLD, NASH, and/or liver fibrosis, which will allow for early treatment, when intervention is likely to have its greatest effect. Selection of an appropriate therapeutic regimen for a given patient may be made based solely on the diagnosis provided by the inventive methods. Alternatively, the physician may also consider other clinical or pathological parameters used in existing methods to diagnose NAFLD, NASH, and/or liver fibrosis and assess its advancement.

The invention further provides a method of treating NAFLD, NASH, liver fibrosis or other associated liver damage or diseases using any drugs, compounds, small molecules, proteins, antibodies, nucleotides, and pharmaceutical compositions thereof, that are capable of reducing circular EVs and/or hepatocyte-derived MPs by internalizing the EVs and/or MPs into endothelial cells so as to reduce pro-angiogenesis or other factors associated with the degree and/or progression of liver damage or diseases. In certain embodiments, the invention provides a method of treating NAFLD, NASH, liver fibrosis or other associated liver damage or diseases using any drugs, compounds, small molecules, proteins, antibodies, nucleotides, and pharmaceutical compositions thereof, that are capable of interacting one or more protein biomarkers expressed and/or detected on the EVs or MPs so as to reducing their expression and/or activity level by inhibiting caspase 3 activation. In certain embodiments, the pharmaceutical composition of the invention comprises an anti-Vanin-1 antibody or an antisense or SiRNA against Vanin-1 protein biomarker.

The invention contemplates any conventional methods for formulation of pharmaceutical compositions as described above. Various additives, known to those skilled in the art, may be included in the formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

Effective dosages and administration regimens can be readily determined by good medical practice and the clinical condition of the individual subject. The frequency of administration will depend on the pharmacokinetic parameters of the active ingredient(s) and the route of administration. The optimal pharmaceutical formulation can be determined depending upon the route of administration and desired dosage. Such formulations may influence the physical state, stability, rate of in vivo release, and rate of in vivo clearance of the administered compounds.

Depending on the route of administration, a suitable dose may be calculated according to body weight, body surface area, or organ size. Optimization of the appropriate dosage can readily be made by those skilled in the art in light of pharmacokinetic data observed in human clinical trials. The final dosage regimen will be determined by the attending physician, considering various factors which modify the action of drugs, e.g., the drug's specific activity, the severity of the damage and the responsiveness of the patient, the age, condition, body weight, sex and diet of the patient, the severity of any present infection, time of administration and other clinical factors.

The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims.

It is to be noted that throughout this application various publications and patents are cited. The disclosures of these publications are hereby incorporated by reference in their entireties into this application in order to describe fully the state of the art to which this invention pertains.

### Example 1

### Hepatocytes Release Microparticles During Lipotoxicity that have Potent Pro-Angiogenic Effects In Vitro and In Vivo Through Vanin-1-Dependent Internalization

The studies in this example provide evidence that fat-overloaded hepatocytes after exposure to the saturated but not unsaturated free fatty acids secrete pro-angiogenic signals. Through several lines of evidence hepatocyte-derived microparticles (MPs) were identified as the putative pro-angiogenic factor both in vitro and in vivo in a process involving caspase 3 activation in hepatocytes and Vanin-1 (VNN1)-dependent internalization of MPs into the endothelial cells. These findings uncover a novel mechanism linking hepatocyte lipotoxicity to angiogenesis and identify putative new therapeutic targets to inhibit angiogenesis and disease progression. More specifically, these findings demonstrated that hepatocyte-derived microparticles (MPs) are critical pro-angiogenic signals that regulate endothelial cell angiogenesis during lipotoxicity.

Supernatants of human and murine hepatocytes exposed to saturated free fatty acids induced marked angiogenesis. These effects were completely abolished after removing vesicles in the supernatants by ultracentrifugation. Further characterization by light scattering, electron microscopy, and FACS analysis identified MPs as the main type of vesicle released by hepatocytes during lipotoxicity. Proteomic analysis of fat-laden hepatocytes-derived MPs identified Vanin-1 (VNN1) as selectively expressed on MPs surface. The release of MPs from hepatocytes was highly regulated and dependent on caspase 3 activation. Isolated hepatocyte-derived MPs induce endothelial cell motility and marked angiogenesis both in vitro and in vivo. This process was dependent on MPs internalization through an interaction of VNN1 with lipid raft domains of the endothelial cells. Furthermore, high levels of hepatocyte derived MPs were detected in two common diet-induced murine models of steatohepatitis and the levels correlated with disease severity. These changes were associated with marked angiogenesis and early fibrosis in the livers of these mice. Genetic inhibition of caspase 3 or VNN1 protected mice from angiogenesis and resulted in a loss of pro-angiogenic effects of MPs ex vivo. These data identify hepatocyte-derived MPs as critical signals that contribute to angiogenesis and liver damage in steatohepatitis and suggest a novel therapeutic target for this condition.

The more detailed results are presented as follows:

Lipid Loaded Hepatocytes Release Factors that Induced Endothelial Cell Migration and Angiogenesis

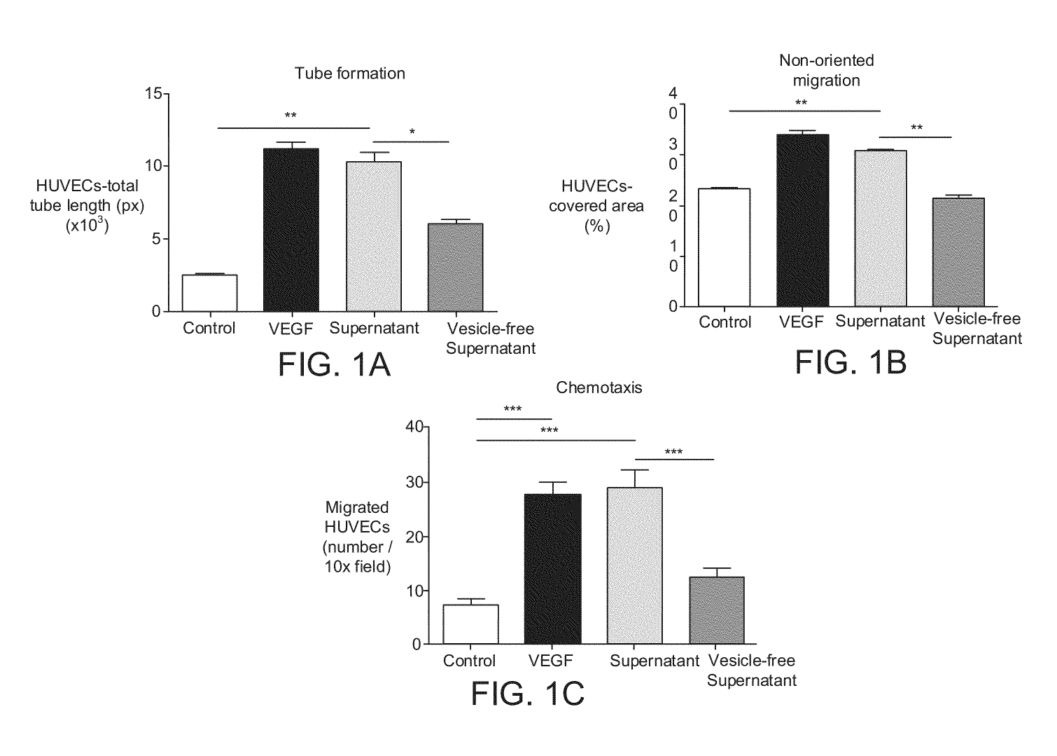

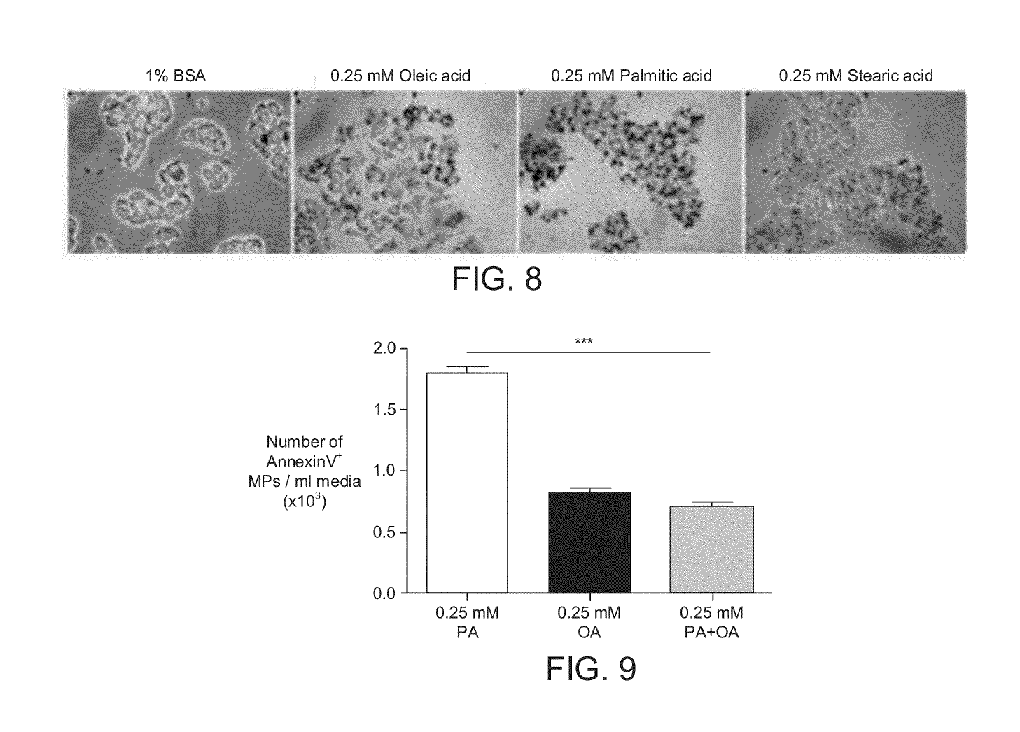

Lipid accumulation in hepatocytes is a critical event in NASH development and is thought to be mainly a result of increased uptake of FFA from the circulation [13]. It has been reported that overloading hepatocytes with saturated FFA such as palmitic acid, rather than monounsaturated or polyunsaturated FFA results in lipotoxicity [14-16]. To determine whether over accumulation of lipotoxic lipids in hepatocytes results in the release of factor/s that induces angiogenesis, cell-free supernatants were initially collected from hepatocytes exposed to the lipotoxic FFAs including palmitic and stearic acid, as well as non-lipotoxic FFA, oleic acid or controls (FIG. 8) and the potential effects on angiogenesis were examined by performing multiple functional assays. For this purpose, HepG2 cells, a well-differentiated human and hepatoma cell line as well as primary rat hepatocytes were incubated with different concentrations of the FFAs for up to 24 hours. Cell-free supernatants from hepatocytes treated with palmitic or stearic acid but not those treated with oleic acid induced marked increase in both tube formation and endothelial cell oriented (chemotaxis) and non-oriented migration to an extent similar to the one induced by pro-angiogenic doses of vascular endothelial growth factor A (VEGF-A), used as positive control (FIGS. 1A-1C). These results strongly suggest that overloading hepatocytes with lipotoxic lipids, results in the formation and release of pro-angiogenic factors.

### The Pro-Angiogenic Effects of Lipid-Loaded Hepatocytes are Mediated by Release of Membrane Vesicles

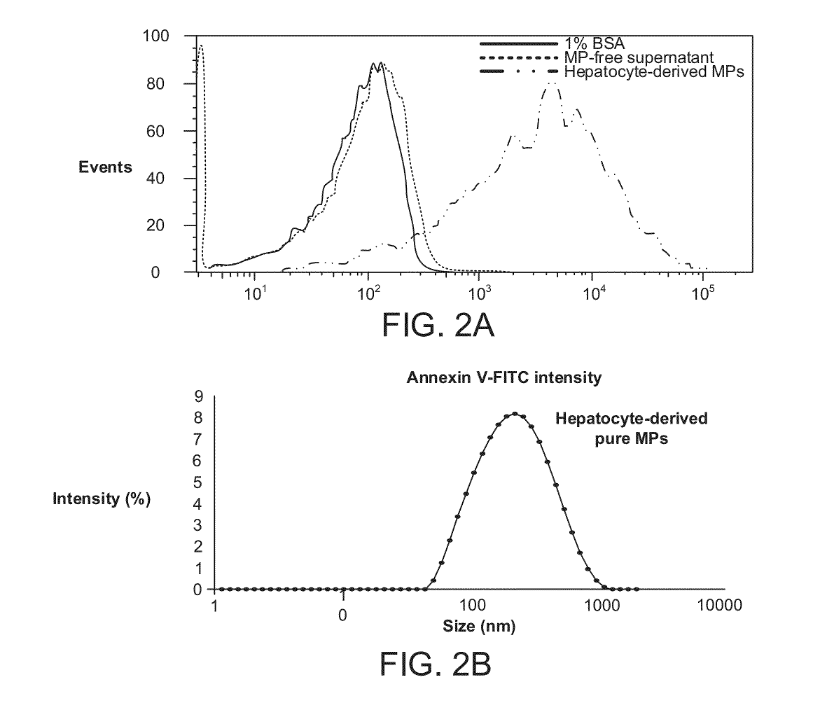

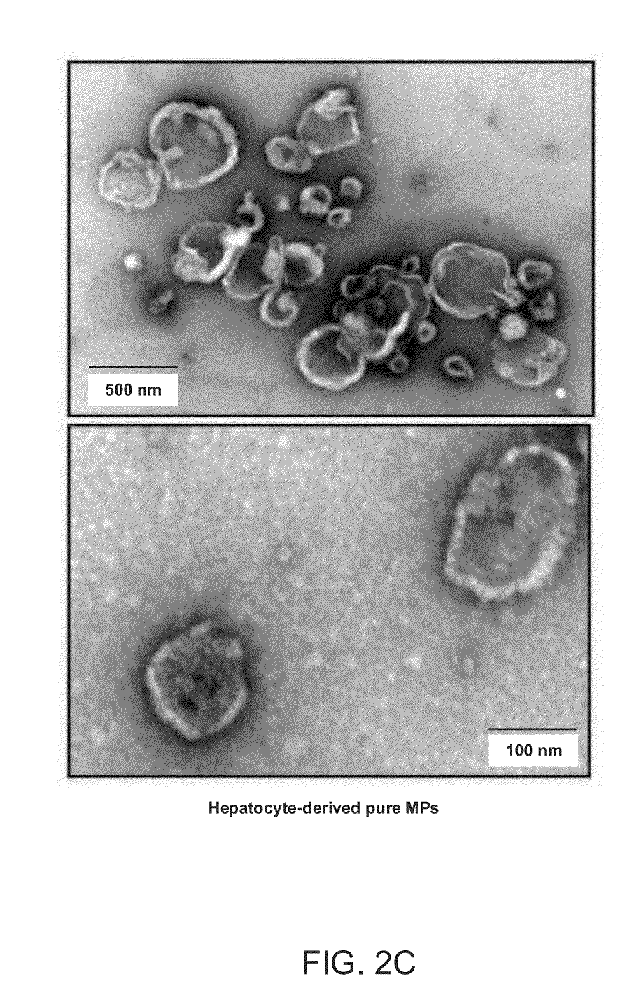

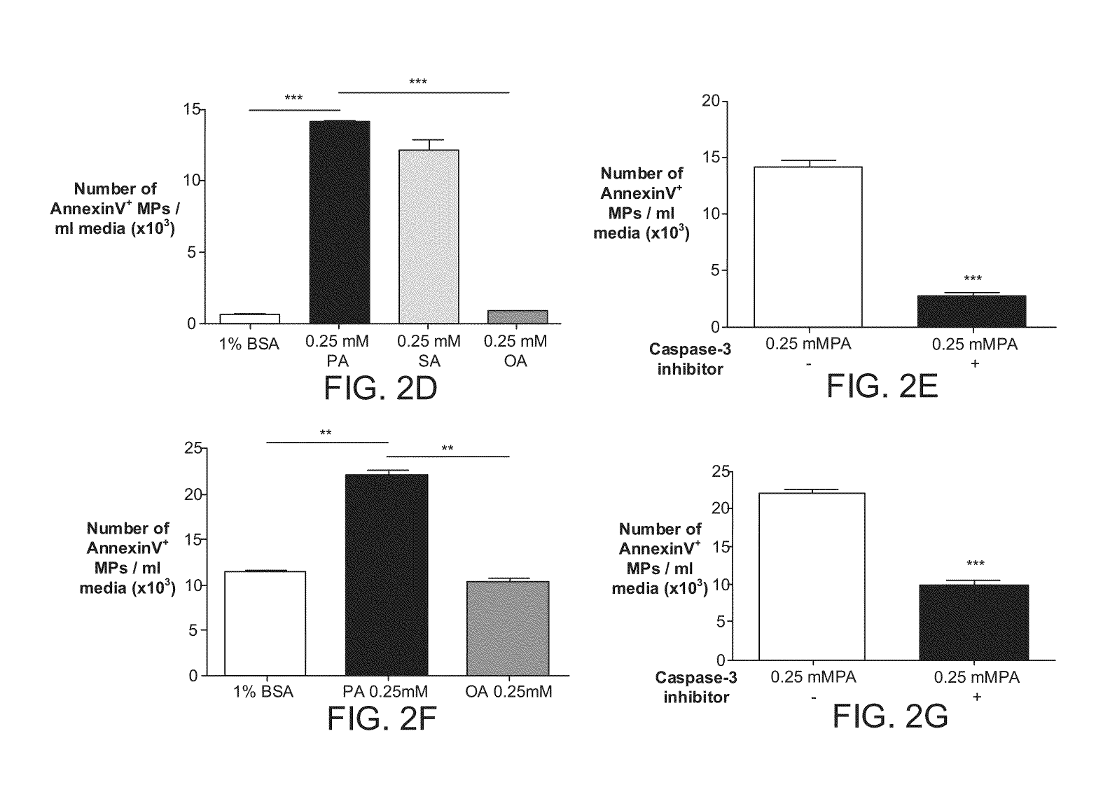

The nature of the pro-angiogenic factor/s released into the supernatants of stressed hepatocytes was then determined. During initial studies focused on physical and biochemical characterization of the pro-angiogenic factor/s, all cell vesicles were removed from palmitic acid treated hepatocyte supernatants (vesicles-free supernatant) by ultracentrifugation. It was found that vesicles-free supernatants completely lost the pro-angiogenic effects on endothelial tube formation and migration in vitro (FIGS. 1A-1C). These findings suggested that the pro-angiogenic activity is present in the membranous precipitate and leads to further characterize the membrane vesicles released by hepatocytes during lipotoxicity. A series of studies was then performed, including dynamic light scattering analysis, transmission electron microscopy (TEM), and FACS analysis that identified microparticles (MPs) as the main membrane vesicle population released by hepatocytes during exposure to FFAs (FIGS. 2A-2C). Indeed, it was observed that the vesicles released in the supernatant have a diameter ranging between 100 and 1,000 nm (mean 220 nm) and morphology corresponding to that of MPs (FIGS. 2B-2C) [17, 18]. Moreover, FACS analysis identified a marked increase in Annexin V positive MPs in the supernatants of palmitic acid treated cells compared to controls (FIGS. 2D and 2F). The release of MPs was specifically linked to lipotoxicity as incubation of hepatocytes with a non-lipotoxic FFA failed to induce any increase in MP formation (FIGS. 2D and 2F), while co-incubation of cells with both lipotoxic and non-lipotixic FFA, palmitic acid and oleic acid respectively, also abrogated the release of MPs (FIG. 9). As MPs generation by other cell types such as platelets has been shown to be regulated by caspase 3 [19, 20], it was next assessed whether suppression of caspase 3 activity would abolish or reduce MPs formation and release by palmitic acid treatment. Co-incubation of palmitic acid-treated hepatocytes with a selective caspase 3 inhibitor drastically reduced the formation and release of MPs (FIGS. 2E and 2G).

Next, a comprehensive characterization of the antigenic composition of hepatocytes-derived MPs was performed. For these studies a proteomic approach by LC-MS/MS analysis was used.

Table 1 lists the highest and repeatedly expressed proteins in hepatocytes-derived MP resident proteins based on LC-MS/MS analysis. Pure hepatocytes-derived microparticles were isolated and processed for a complete proteomics analysis as described in the ‘Methods’ paragraph. A short list of the highest and consistently expressed proteins are listed in the table with the corresponding uniprot accession code, sequence of coverage (%), number of peptides and molecular mass based on GO Consortium. For this study Vanin-1 (VNN1) was focused on as novel ectoenzyme which plays an important role in cell adhesion and migration.

TABLE 1
Identification of the highest expressed hepatocytes-
derived MPs proteins based on LC-MS/MS data

| | | Sequence | N. | MW |
|---|---|---|---|---|
| Protein Name | Accession Code | coverage % | peptides | Kda |
| Maltase-glucoamylase | MGA_HUMAN | 17.99 | 20 | 196.9 |
| Ceruloplasmin | CERU_HUMAN | 35.96 | 38 | 122.2 |
| precursor | | | | |
| Amine oxidase, | AOC3_HUMAN | 15.07 | 10 | 84.6 |
| copper containing 3 | | | | |
| Apolipoprotein E | APOE_HUMAN | 30.6 | 6 | 36.1 |
| precursor | | | | |
| Vitamin D-binding | VTDB_HUMAN | 12.66 | 7 | 52.9 |
| protein precursor | | | | |
| Isocitrate | IDHC_HUMAN | 12.56 | 4 | 46.6 |
| dehydrogenase 1, | | | | |
| soluble | | | | |
| fumarylacetoacetate | FAAA_HUMAN | 18.62 | 5 | 46.3 |
| hydrolase | | | | |
| Vanin-1 | VNN1_HUMAN | 13.06 | 3 | 57 |
| Transforming growth | BGH3_HUMAN | 10.25 | 3 | 74.6 |
| factor, beta-induced | | | | |

Table 2 provides an identification of the whole hepatocyte-derived microparticles proteins based on the LC-MS/MS-derived sequences. Pure hepatocyte-derived microparticles were isolated and processed for a complete proteomics analysis as described in the ‘Methods’ paragraph. The proteins detected are listed in the table with the corresponding uniprot accession code, number of peptides, biological function and cellular localization based on GO Consortium.

TABLE 2
IdentificationIdentification of the Whole hepatocytes-derived
microparticles proteins based on the LC-MS/MS-derived sequences

| | | | | | |
|---|---|---|---|---|---|
| | | Number | | | |
| | Accession | of | Molecular | | Cellular |
| Protein name | code | peptides | function | Biological process | localization |

CYTOSKELETON/VESICULATION/ENDOCYTOSIS

| | | | | | |
|---|---|---|---|---|---|
| Tubulin, alpha, ubiquitous | gi\|57013276 | 2 | Nucleotide | Cytoskeleton | Cytoplasm |
| | | | binding | organisation | |
| Tubulin, alpha 1a | gi\|17986283 | 2 | Nucleotide | Mitosis/transport/ | Cytoplasm |
| | | | binding | protein folding/ | |
| | | | | cytoskeleton | |
| | | | | organisation | |
| Tubulin alpha 6 | gi\|14389309 | 2 | Nucleotide | Cytoskeleton | Cytoplasm |
| | | | binding | organisation | |
| Tubulin, alpha 3e | gi\|46409270 | 1 | Nucleotide | Cytoskeleton | Cytoplasm |
| | | | binding | organisation | |
| Tubulin, alpha 3c | gi\|17921993 | 1 | Nucleotide | Cytoskeleton | Cytoplasm |
| | | | binding | organisation | |
| Tubulin, alpha 3d | gi\|156564363 | 1 | Nucleotide | Cytoskeleton | Cytoplasm |
| | | | binding | organisation | |
| Tubulin, beta, 2 | gi\|5174735 | 1 | Nucleotide | Cytoskeleton | Cytoplasm |
| | | | binding | organisation | |
| Tubulin, beta | gi\|29788785 | 1 | Nucleotide | Cytoskeleton | Cytoplasm |
| | | | binding | organisation | |
| Tubulin, beta 8 | gi\|42558279 | 1 | Nucleotide | Cytoskeleton | Cytoplasm |
| | | | binding | organisation | |
| Tubulin, beta 2B | gi\|29788768 | 1 | Nucleotide | Cytoskeleton | Cytoplasm |
| | | | binding | organisation | |
| Tubulin, beta 4 | gi\|21361322 | 1 | Nucleotide | Cytoskeleton | Cytoplasm |
| | | | binding | organisation | |
| Tubulin, beta polypeptide 4, | gi\|55770868 | 1 | Nucleotide | Cytoskeleton | Cytoplasm |
| member Q | | | binding | organisation | |
| Tubulin, beta 6 | gi\|14210536 | 1 | Nucleotide | Cytoskeleton | Cytoplasm |
| | | | binding | organisation | |

ENZYMES/METABOLIC PROCESSES

| | | | | | |
|---|---|---|---|---|---|
| Albumin precursor | gi\|4502027 | 42 | Protein binding | Transport/response to | Extracellular |
| | | | | stress/lipid metabolism | space/ |
| | | | | | cytoplasm |
| Maltase-glucoamylase | gi\|4758712 | 25 | Glucosidase | Carbohydrate | Plasma |
| | | | | metabolism | membrane |
| Ceruloplasmin precursor | gi\|4557485 | 55 | Ferroxidase | Ion transport | Extracellular |
| | | | | | space |
| Apolipoprotein E precursor | gi\|4557325 | 6 | Lipid/protein | Lipid metabolism | Extracellular |
| | | | binding | | space/ |
| | | | | | cytoplasm |
| Amine oxidase, copper | gi\|4502119 | 12 | Oxidase/binding | Catecholamine | Cytoplasm/ |
| containing 3 precursor | | | | metabolism/adhesion/ | plasma |
| | | | | inflammation | membrane |
| Vitamin D-binding protein | gi\|32483410 | 7 | Protein binding | Vitamin D transport | Extracellular |
| precursor | | | | | space |
| Isocitrate dehydrogenase 1 | gi\|28178825 | 4 | Dehydrogenase | Isocitrate oxidative | Cytoplasm/ |
| (NADP+), soluble | | | | decarboxylation | mitochondrion/ |
| | | | | | peroxisome |
| Fumarylacetoacetate | gi\|4557587 | 5 | Hydrolase | Tyrosine catabolism | Cytoplasm |
| hydrolase | | | | | |
| Apolipoprotein A-I | gi\|4557321 | 3 | Lipid/protein | Lipid metabolism/ | Extracellular |
| preproprotein | | | binding | platelet activation/ | space/ |
| | | | | endothelial cell | cytoplasm/ |
| | | | | proliferation | plasma |
| | | | | | membrane |
| Vanin-1 precursor | gi\|4759312 | 3 | Hydrolase/GPI | Inflammatory response/ | Plasma |
| | | | anchor binding | anti-apoptosis/adhesion/ | membrane |
| | | | | cell migration | |
| Eukaryotic translation | gi\|4503471 | 2 | Nucleotide | Transcription/ | Cytoplasm/ |
| elongation factor 1 alpha 1 | | | binding | translation regulation | Nucleus |
| Clustein isoform 2 | gi\|42740907 | 2 | ATPase/ | Apoptosis regulation | Extracellular |
| | | | misfolded protein | | space/ |
| | | | binding | | cytoplasm/ |
| | | | | | mitochondrion |
| Clusterin isoform 1 | gi\|42716297 | 2 | ATPase/ | Apoptosis regulation | Extracellular |
| | | | misfolded protein | | space/ |
| | | | binding | | cytoplasm/ |
| | | | | | mitochondrion |
| Haptoglobin | gi\|4826762 | 2 | Catalytic activity | Hemoglobin binding/ | Extracellular |
| | | | | catabolic process | space |
| Haptoglobin-related protein | gi\|45580723 | 1 | Catalytic activity | Hemoglobin binding | Extracellular |
| | | | | | space |
| Glyceraldehyde-3-phosphate | gi\|7669492 | 3 | Dehydrogenase | Glycolisis | Cytoplasm |
| dehydrogenase | | | | | |
| Dipeptidylpeptidase IV | gi\|18765694 | 1 | Aminopeptidase | Endothelial cell migration/ | Plasma |
| | | | | adhesion/proteolysis | membrane/ |
| | | | | | Golgi |
| | | | | | apparatus |
| Alpha 1 globin | gi\|4504347 | 1 | Transporter | Oxygen transport | Extracellular |
| | | | | | space |
| Alpha 2 globin | gi\|4504345 | 1 | Transporter | Oxygen transport | Extracellular |
| | | | | | space |
| Plasma glutamate | gi\|7706387 | 3 | Peptidase | Proteolysis | Cytoplasm/ |
| carboxypeptidase | | | | | plasma |
| | | | | | membrane/ |
| | | | | | nucleus |
| Eukaryotic translation | gi\|4503475 | 1 | Nucleotide | Translation/anti- | Cytoplasm/ |
| elongation factor 1 alpha | | | binding | apoptosis | nucleus |
| Hornerin | gi\|57864582 | 3 | Isomerase | Tryptophan metabolic | Extracellular |
| | | | | process | space |
| Aspartate aminotransferase | gi\|4504067 | 1 | Transaminase | Amino acid degradation | Cytoplasm |
| 1 | | | | and biosynthesis | |
| Lysozyme precursor | gi\|4557894 | 1 | Hydrolase/ | Inflammatory response/ | Extracellular |
| Serine (or cysteine) | | | Lysozyme activity | cytolysis | space |
| proteinase inhibitor, clade A, | gi\|4507377 | 1 | Protein binding | Regulation of proteolysis | Extracellular |
| member 7 | | | | | space |

RIBONUCLEOPROTEINS

| | | | | | |
|---|---|---|---|---|---|
| Ribosomal protein S10 | gi\|4506679 | 1 | Protein binding | Translation | Nucleus |

EXTRACELLULAR MATRIX

| | | | | | |
|---|---|---|---|---|---|
| Transforming growth factor, | gi\|4507467 | 3 | Extracellular | Angiogenesis/cell | Extracellular |
| beta-induced, 68 kDa | | | matrix binding | adhesion | space |
| Vitronectin precursor | gi\|88853069 | 2 | Extracellular | Cell-matrix adhesion/ | Extracellular |
| | | | matrix binding | migration/integrin | space |
| | | | | binding | |

NUCLEOSOMES

| | | | | | |
|---|---|---|---|---|---|
| Histone cluster 2, H4b | gi\|77539758 | 2 | DNA binding | Nucleosome assembly | Nucleus |
| Histone cluster 2, H4a | gi\|4504323 | 2 | DNA binding | Nucleosome assembly | Nucleus |
| Histone cluster 1, H4i | gi\|4504321 | 2 | DNA binding | Nucleosome assembly | Nucleus |
| Histone cluster 1, H4l | gi\|4504317 | 2 | DNA binding | Nucleosome assembly | Nucleus |
| Histone cluster 1, H4e | gi\|4504315 | 2 | DNA binding | Nucleosome assembly | Nucleus |
| Histone cluster 1, H4b | gi\|4504313 | 2 | DNA binding | Nucleosome assembly | Nucleus |
| Histone cluster 1, H4h | gi\|4504311 | 2 | DNA binding | Nucleosome assembly | Nucleus |
| Histone cluster 1, H4c | gi\|4504309 | 2 | DNA binding | Nucleosome assembly | Nucleus |
| Histone cluster 1, H4k | gi\|4504307 | 2 | DNA binding | Nucleosome assembly | Nucleus |
| Histone cluster 1, H4f | gi\|4504305 | 2 | DNA binding | Nucleosome assembly | Nucleus |
| Histone cluster 1, H4d | gi\|4504303 | 2 | DNA binding | Nucleosome assembly | Nucleus |
| Histone cluster 1, H4a | gi\|4504301 | 2 | DNA binding | Nucleosome assembly | Nucleus |
| Histone cluster 4, H4 | gi\|28173560 | 2 | DNA binding | Nucleosome assembly | Nucleus |
| Histone cluster 1, H4j | gi\|11415030 | 2 | DNA binding | Nucleosome assembly | Nucleus |
| Histone cluster 2, H2be | gi\|4504277 | 2 | DNA binding | Nucleosome assembly | Nucleus |
| Histone cluster 3, H2bb | gi\|28173554 | 2 | DNA binding | Nucleosome assembly | Nucleus |
| Histone cluster 1, H2bj | gi\|20336754 | 2 | DNA binding | Nucleosome assembly | Nucleus |
| Histone cluster 1, H2bo | gi\|16306566 | 2 | DNA binding | Nucleosome assembly | Nucleus |
| Histone cluster 1, H2bb | gi\|10800140 | 2 | DNA binding | Nucleosome assembly | Nucleus |
| Histone cluster 2, H2bf | gi\|66912162 | 1 | DNA binding | Nucleosome assembly | Nucleus |
| Histone cluster 1, H2bi | gi\|4504271 | 1 | DNA binding | Nucleosome assembly | Nucleus |
| Histone cluster 1, H2bh | gi\|4504269 | 1 | DNA binding | Nucleosome assembly | Nucleus |
| Histone cluster 1, H2bf | gi\|4504265 | 1 | DNA binding | Nucleosome assembly | Nucleus |
| Histone cluster 1, H2bm | gi\|4504263 | 1 | DNA binding | Nucleosome assembly | Nucleus |
| Histone cluster 1, H2bn | gi\|4504261 | 1 | DNA binding | Nucleosome assembly | Nucleus |
| Histone cluster 1, H2bl | gi\|4504259 | 1 | DNA binding | Nucleosome assembly | Nucleus |
| Histone cluster 1, H2bg | gi\|4504257 | 1 | DNA binding | Nucleosome assembly | Nucleus |
| Histone cluster 1, H2be | gi\|21396484 | 1 | DNA binding | Nucleosome assembly | Nucleus |
| Histone cluster 1, H2bc | gi\|21166389 | 1 | DNA binding | Nucleosome assembly | Nucleus |
| Histone cluster 1, H2bd | gi\|20336752 | 1 | DNA binding | Nucleosome assembly | Nucleus |
| Histone cluster 1, H2bk | gi\|18105048 | 1 | DNA binding | Nucleosome assembly | Nucleus |

OTHER PROTEINS

| | | | | | |
|---|---|---|---|---|---|
| Fibrinogen, alpha | gi\|4503689 | 1 | Protein binding | Coagulation | Extracellular |
| polypeptide isoforrn alpha-E | | | | | space |
| preproprotein | | | | | |
| Fibrinogen, alpha | gi\|11761629 | 1 | Protein binding | Coagulation | Extracellular |
| polypeptide isoform alpha | | | | | space |
| preproprotein | | | | | |
| Chemokine (C-C motif) | gi\|4759076 | 1 | Chemokine | Chemotaxis/ | Extracellular |
| ligand 20 | | | | inflammation/signal | space |
| | | | | transduction | |
| Growth differentiation factor | gi\|153792495 | 1 | Growth factor | Inflammation response/ | Cytoplasm |
| 15 | | | (TGF-β family) | apoptosis | |

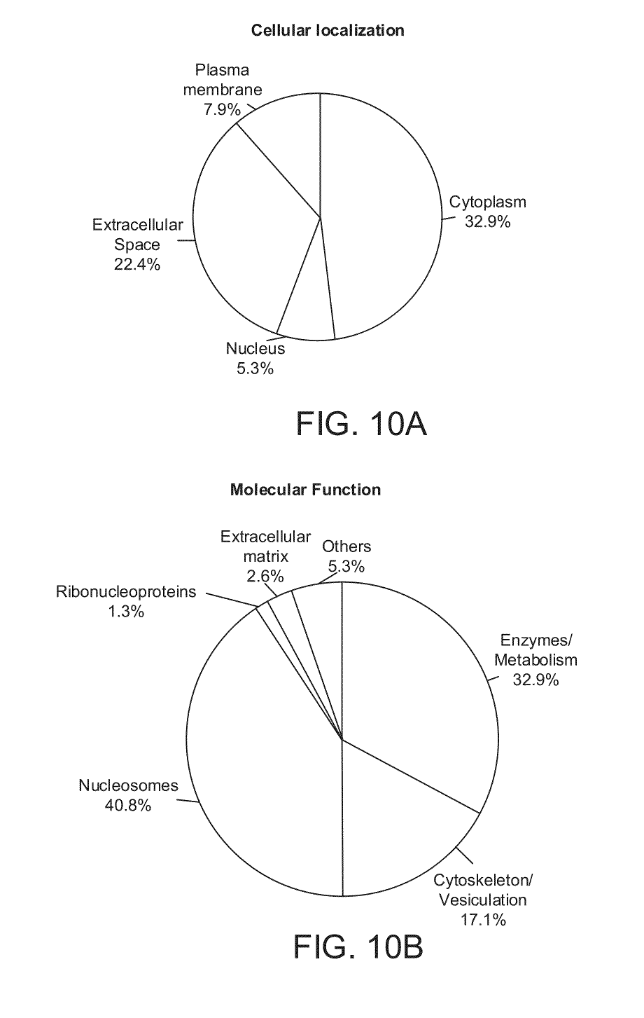

From the newly discovered proteins, hepatocyte-derived MPs carry in order of abundance: cytosolic, extracellular, plasma membrane, and nuclear proteins (FIG. 10). These data demonstrate that during lipotoxicity, MPs carrying a unique antigenic composition are released from hepatocytes in a regulated process dependent on caspase 3 activation and that these hepatocyte-derived MPs may represent critical mediators of the angiogenic effects present in the supernatants of lipid-loaded hepatocytes.

Hepatocyte-Derived MPs are a Novel Pro-Angiogenic Factor that Results in Endothelial Cell Migration and Angiogenesis Both In Vivo and In Vitro in a Process Requiring Vanin-1-Dependent Internalization.

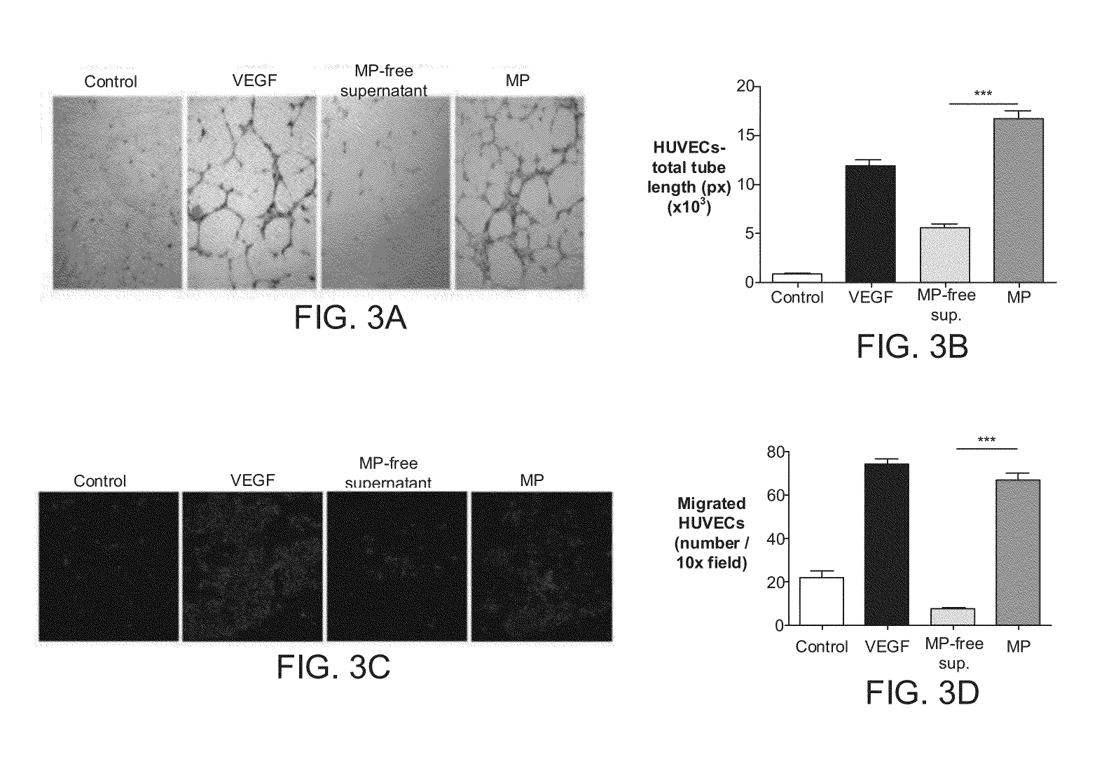

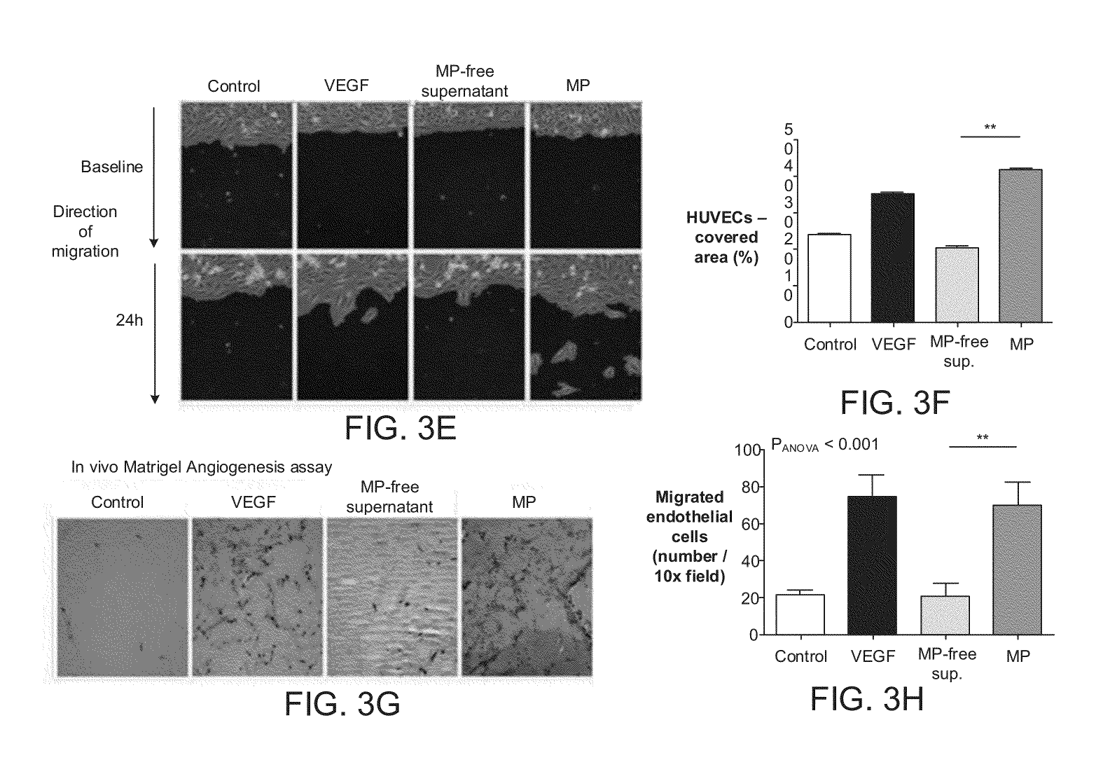

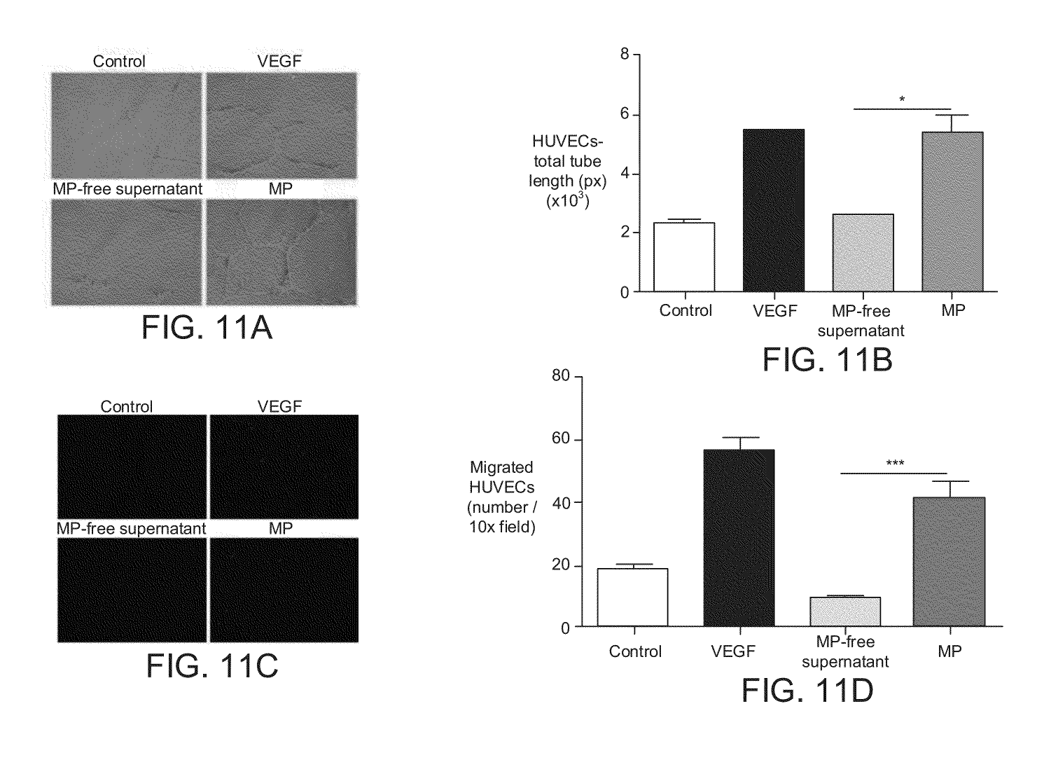

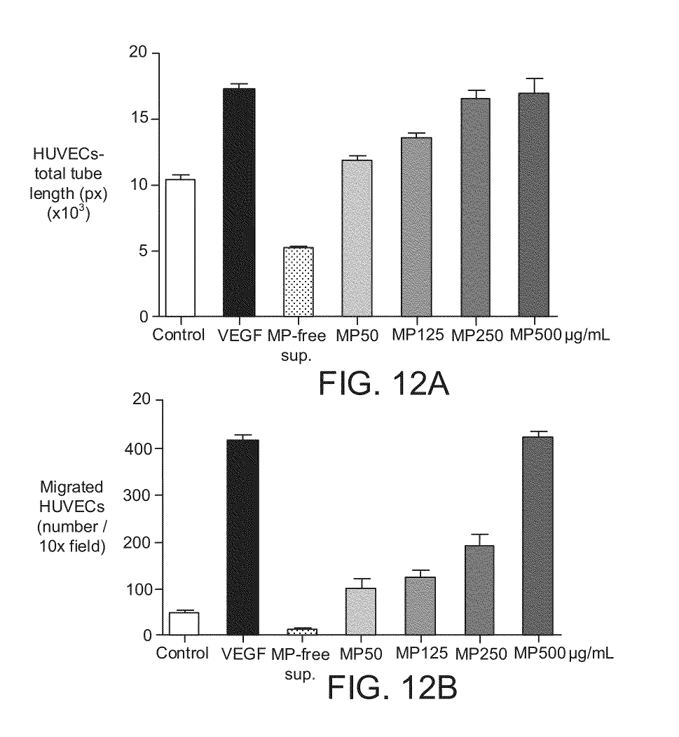

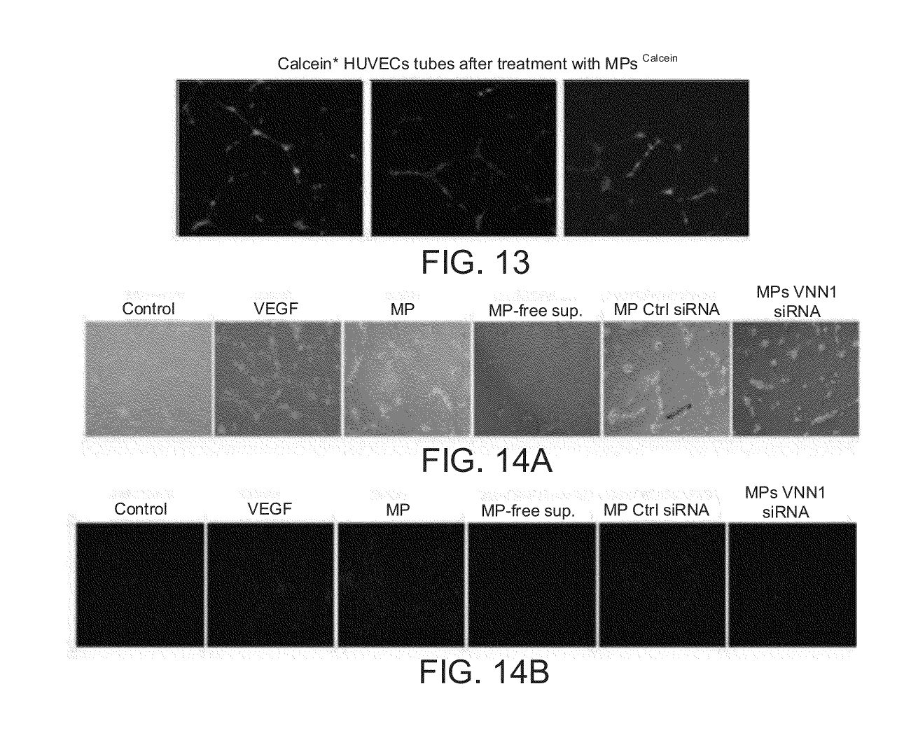

The findings showing that supernatants of lipid-loaded hepatocytes can induce key events involved in angiogenesis and that these pro-angiogenic effects were completely abrogated when used vesicles-free supernatants led to further explore the role of hepatocyte derived MPs in these processes. Isolated hepatocyte-derived MPs from either HepG2 cells (FIGS. 3(A)-3(C)) or primary rat hepatocytes (FIGS. 11(A)-11(D)) treated with palmitic acid resulted in marked increase in endothelial cell migration and tube formation activity in vitro. Further kinetic studies of the response of endothelial cells to MP incubation demonstrated a dose dependent effect of MPs on angiogenesis (FIGS. 12(A) and 12(B)). Moreover, in the tube formation assays, a large number of MPs could be detected inside the tubular structures suggesting that MPs are internalized by endothelial cells (FIG. 13). To elucidate whether hepatocytes-derived MPs could also contribute to angiogenesis in vivo, hepatocytes derived MPs mixed with Matrigel were intradermically injected into athymic BALB/c nude mice. Fourteen days post initial injection Matrigel plugs were removed, fixed and embedded. Similarly to the effects found in in vitro experiments, hepatocyte-derived MPs induced marked in vivo angiogenesis (FIG. 3(D)), to a similar extent or even exceeding the one induced by injecting VEGF.

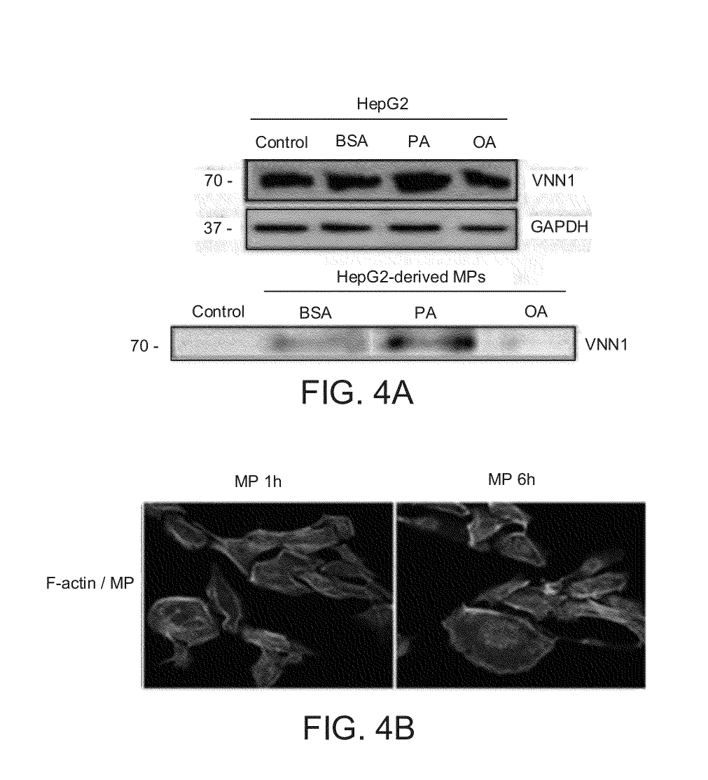

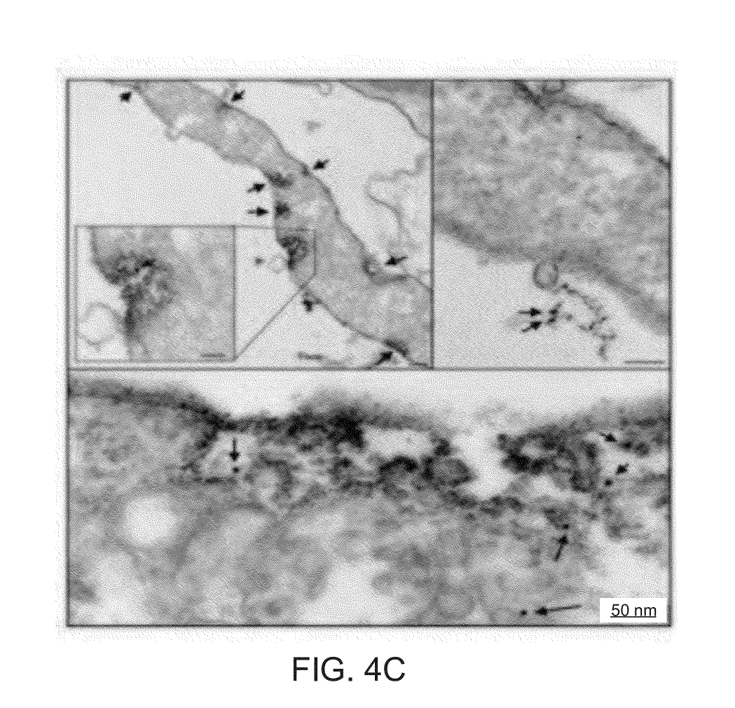

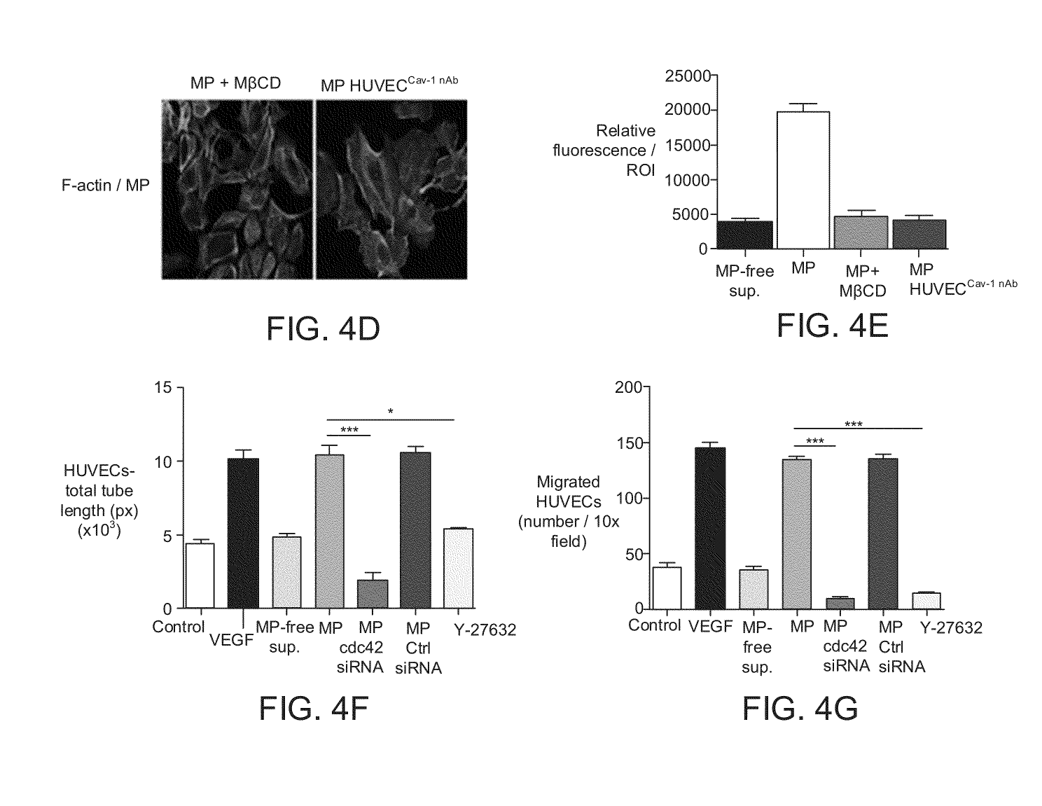

Based on these observations and the findings on the proteomic analysis of microparticles, Vanin-1 (VNN1) was focused on as a plausible functional candidate. Indeed, VNN1 is a novel cell surface expressed enzyme or ectoenzyme that contains a glycosylphosphatidylinositol (GPI)-anchored cleavage site on residue 491. VNN1 is expressed primarily in liver, kidney epithelia, and intestine [21] and has been recently linked to important roles in cell adherence and migration [22]. Western blot analysis confirmed that VNN1 was expressed in hepatocytes and in MPs from palmitic acid but not oleic acid treated cells (FIG. 4A). Next, it was demonstrated that MPs are internalized into the endothelial cells, mainly after 6 h of incubation (FIG. 4B). In order to elucidate how MPs are internalized into the endothelial cells, the role of VNN1 in cell adhesion and affinity with the lipid raft domains on the plasma membrane of the target cells was further investigated. Lipid raft-mediated internalization, cell function, transduction pathways and affinity with GPI-anchored proteins, has been previously described [23, 24]. Particularly, caveolae, flask-like invaginations of the plasma membrane and a subset of lipid rafts, enriched in cholesterol, glycosphingolipids and the structural proteins caveolins are able to modulate cell activity and extracellular vesicles uptake [25-27]. It was found that MPs internalization is mediated by the interaction of VNN1 with lipid raft domains of the endothelial cells. Live endothelial cells were pre-incubated with cholera toxin B HRP conjugate to label lipid raft, as previously reported [28] followed by VNN1-gold labeled MPs incubation. Immunogold-electron microscopy showed a co-localization of VNN1 positive MPs and lipid raft as well as evidence of lipid raft-mediated uptake and trafficking of MPs into the endothelial cells (FIG. 4(C)). Further evidence showed that cholesterol depletion using a no cytotoxic exposure time and concentration of Methyl-β-cyclodextrin (MβCD, previously shown to disrupt lipid raft/caveolae [29]) and using a neutralizing antibody for caveolin-1, resulted in a marked reduction of MPs internalization into the endothelial cells (FIG. 4(D)). Importantly, it was found that the MP-mediated angiogenic effect of endothelial cells could involve the activation of small GTPases, such as RhoA and cdc42, as previously described for the intracellular signal transduction through the lipid raft/GPI-anchored proteins [30-32]. Indeed, specific inhibition of RhoA by the pharmacological inhibitor Y-27632 and the knockdown of cdc42 in endothelial cells resulted in a significant decrease in tube formation and chemotaxis (FIGS. 4E and 4F).

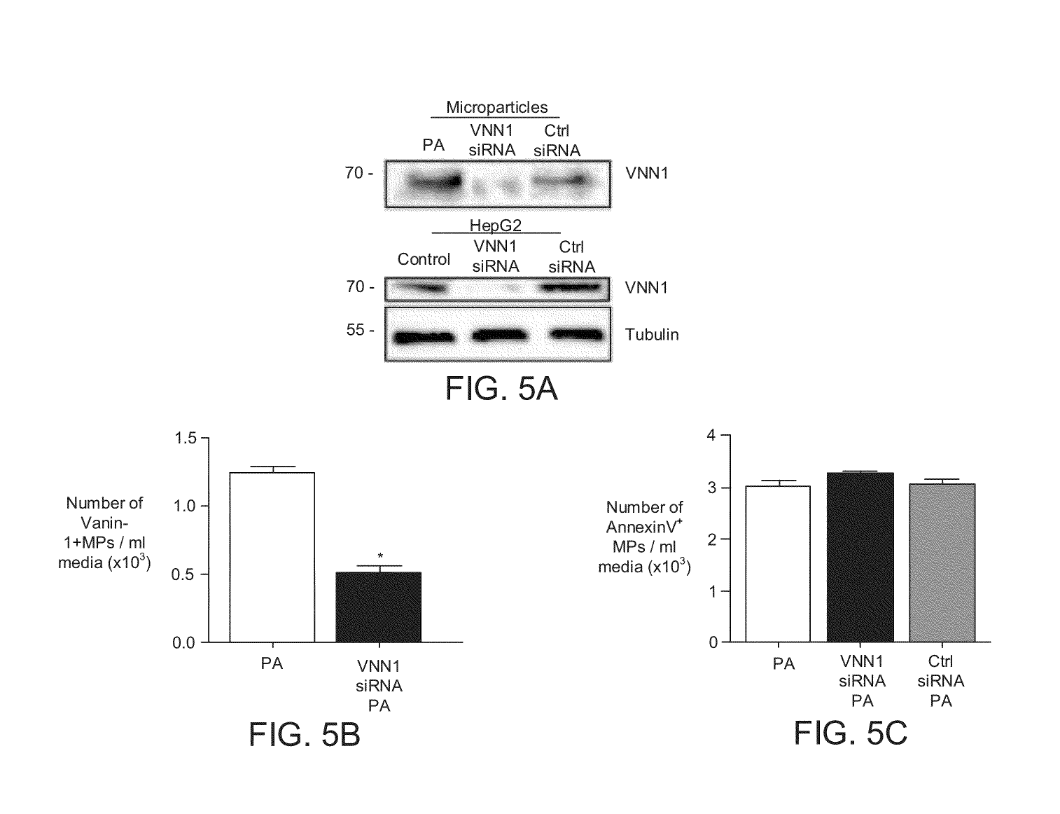

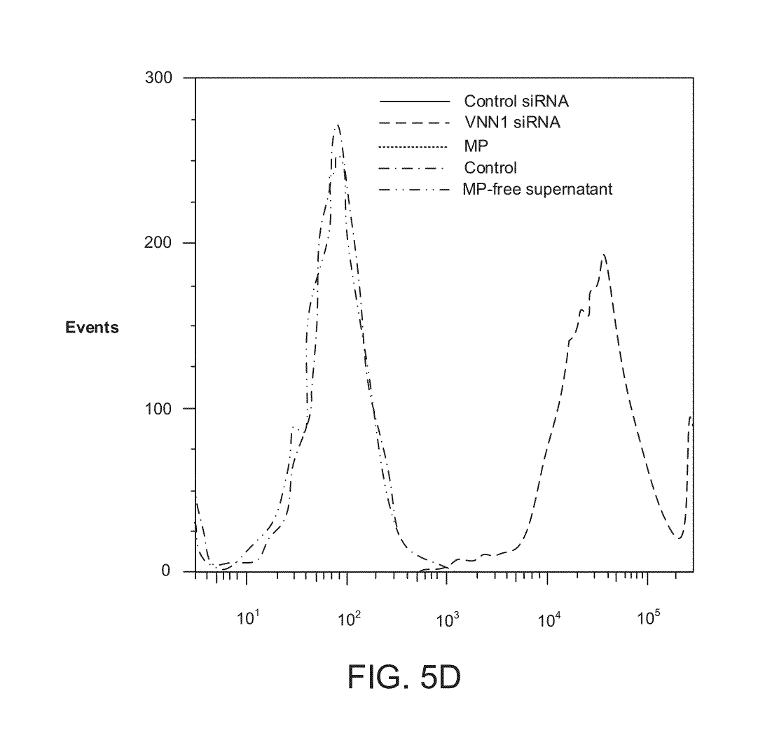

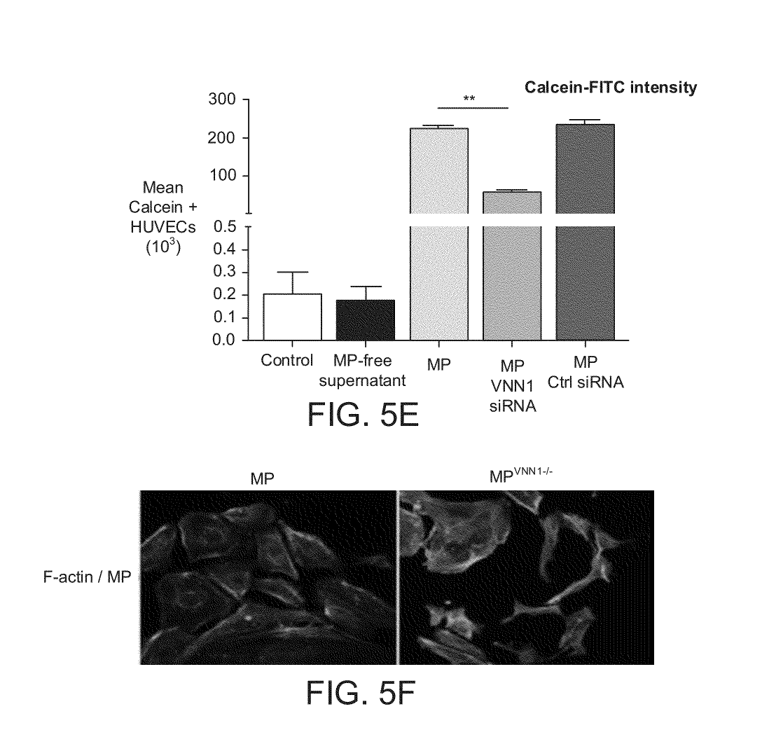

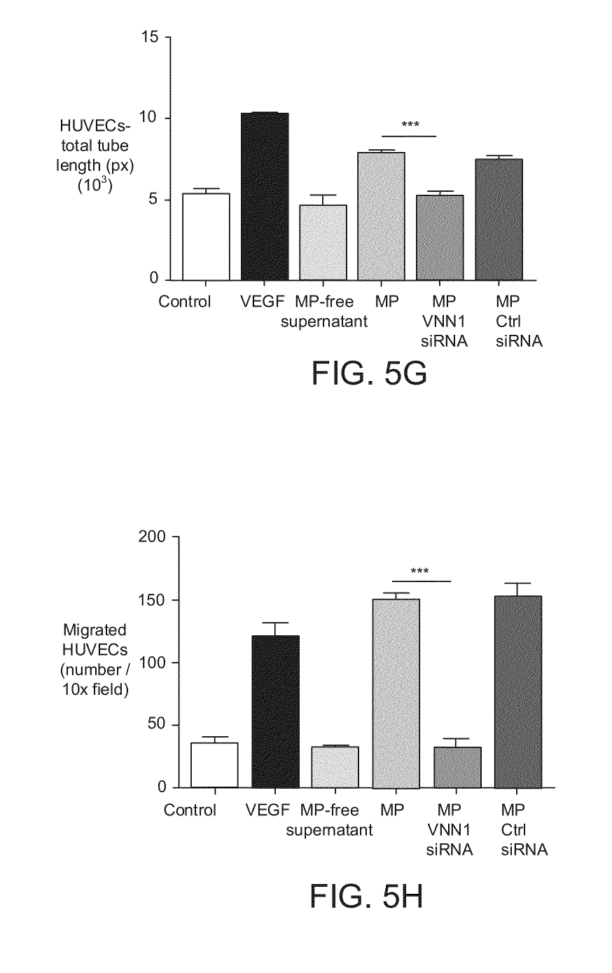

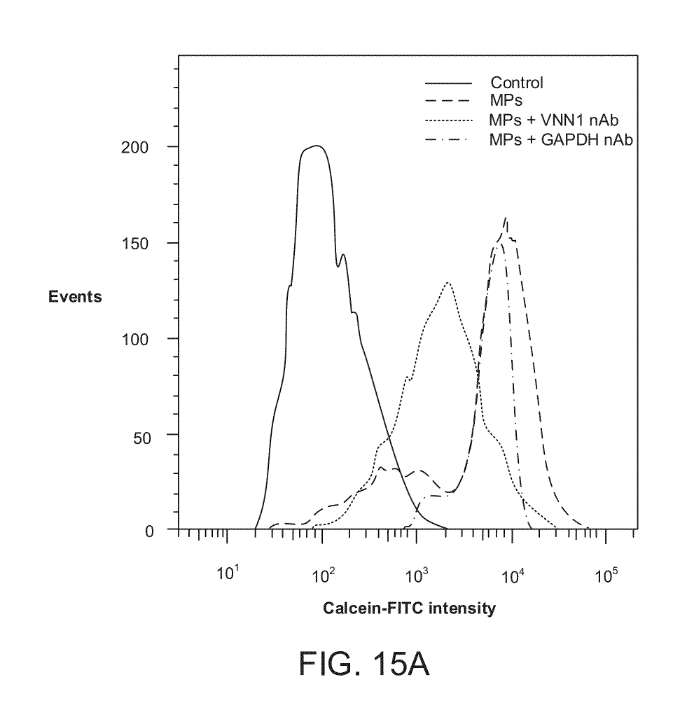

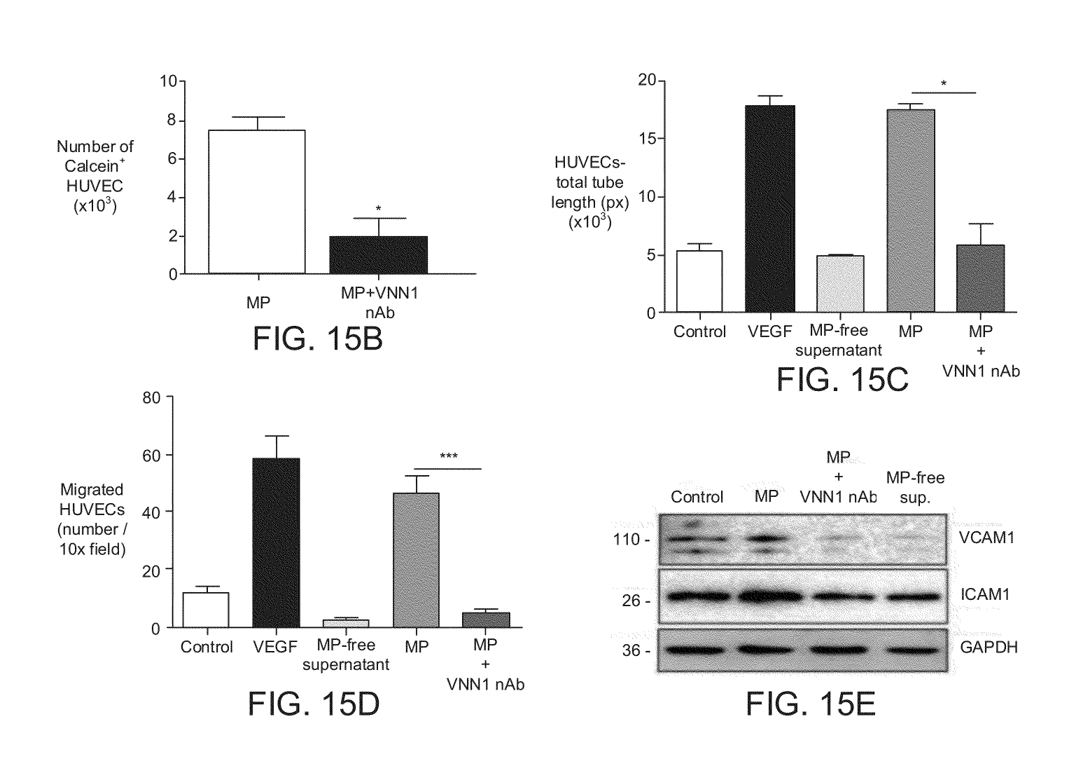

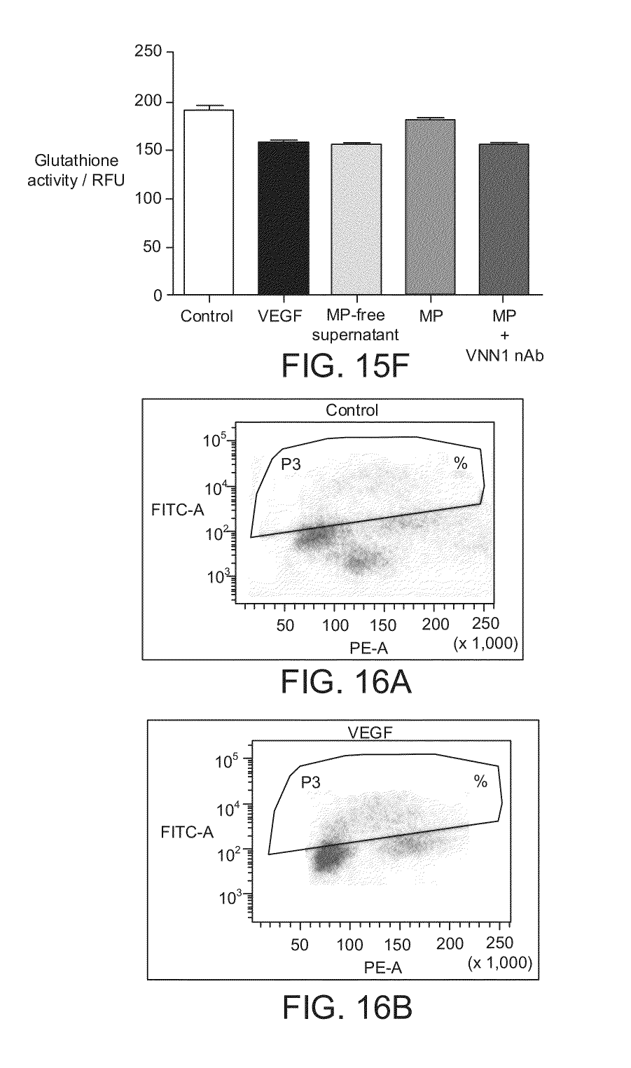

The crucial role of VNN1 in the internalization of MPs was further investigated in vitro by knocking down and neutralizing VNN1 expression in the MPs. For this purpose, HepG2 cells were treated with small interfering RNA (siRNA) for VNN1 or with control siRNA (Ctrl siRNA) and incubated with 0.25 mM of palmitic acid 48 h post-transfection. This approach resulted in a significant reduction of VNN1 expression in both HepG2 and corresponding MPs released after exposure to FFA (FIGS. 5A-5B). While the knockdown of VNN1 did not affect the number of MPs released after exposure to palmitic acid (FIG. 5(C)), interestingly, genetic knockdown of VNN1 expression on MPs external leaflet, significantly reduced MPs internalization by endothelial cells (FIGS. 5(D), 5(E) and 5(F)) and more importantly, the pro-angiogenic effects on endothelial cell migration and tube formation (FIGS. 5(G) and 5(H), and 14(A) and 14(B)). In order to obtain further evidence on the role of VNN1, VNN1 was blocked on the MPs external leaflet by using a specific neutralizing VNN1 antibody. Similar to the findings with the siRNA approach for VNN1, the incubation with VNN1 neutralizing antibody resulted in a marked suppression of MPs internalization into the endothelial cells (HUVECs) and, more importantly of the pro-angiogenic effect of MPs (FIG. 15A-15E). The pantetheinase activity of VNN1 in MPs that result in reduction of intracellular glutathione in the target cell was further analyzed. No significant alterations of glutathione in the endothelial cells treated with VNN1-expressing MPs were observed (FIG. 15F).

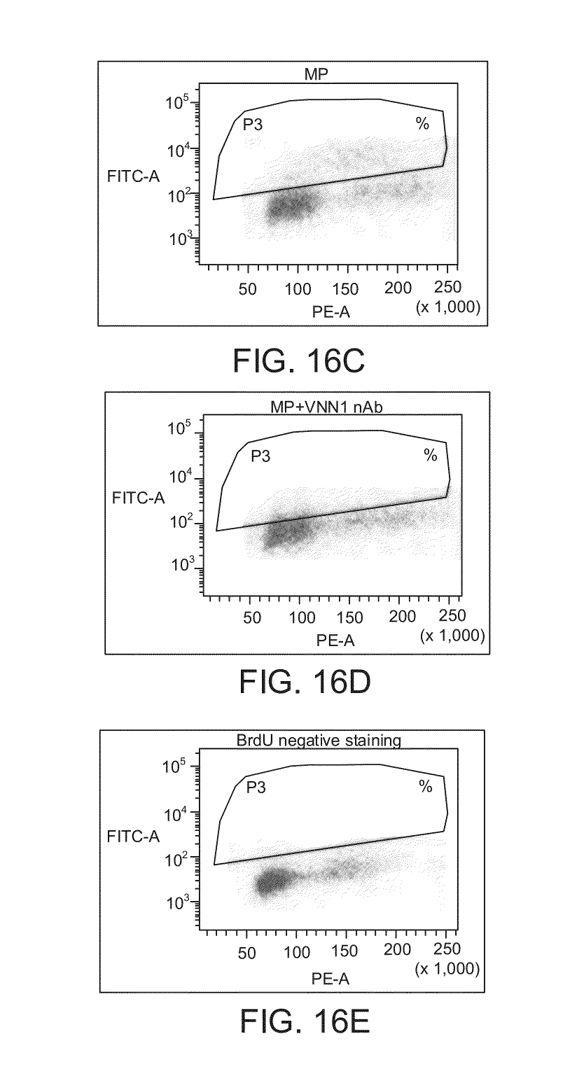

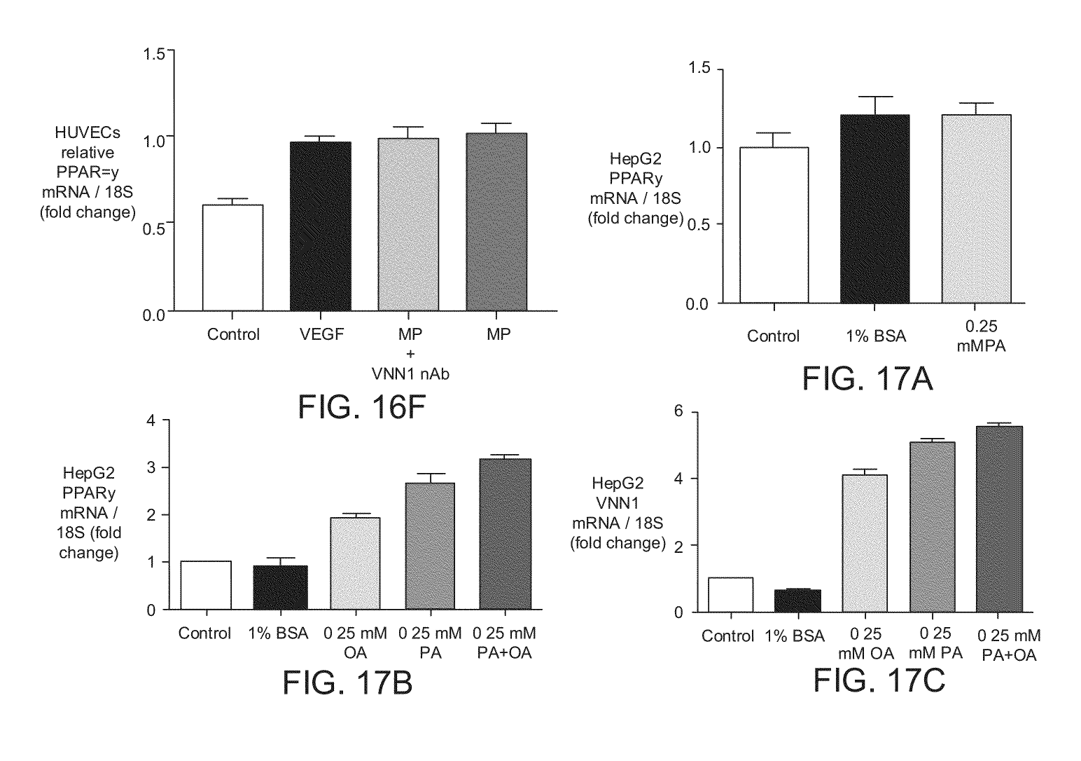

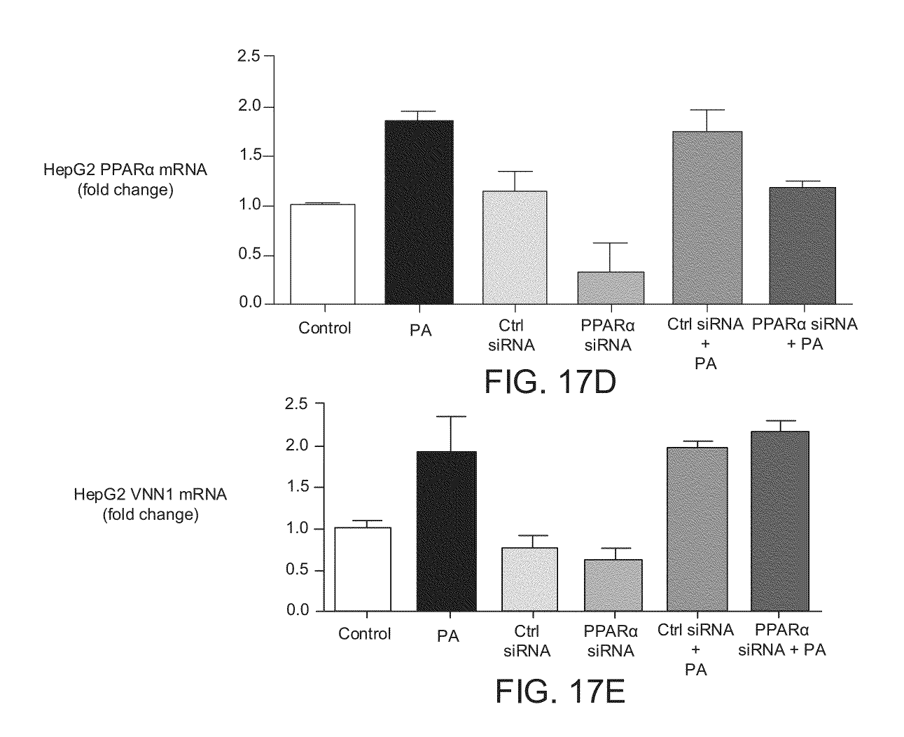

Because VNN1 has been recently shown to potentially modulate vascular smooth muscle cells (SMCs) proliferation partly via peroxisome proliferator-activated receptor gamma (PPARγ) [33], whether internalized VNN1 positive MPs could potentially influence endothelial cell activation through PPARγ was then investigated. It was found that the pro-angiogenic effects of MPs on endothelial cells after exposure for 16 hours did not involve modulation of PPARγ expression and were not caused by significant stimulation of endothelial cell proliferation (FIGS. 16A and 16B). This is consistent with previous reports demonstrating that cells organize themselves into tube-like structures mainly by changing their shape and establishing contacts with neighboring cells but do not significantly proliferate during tube formation and migration [34]. Also, recent studies reported that VNN1 up-regulation during lipotoxicity may depend on PPARs factors [35]. In the instant study, the exposure of HepG2 to the saturated and unsaturated FFA up-regulates the expression of PPARα and VNN1, but not of PPARγ (FIGS. 17A-17C). In order to investigate whether the up-regulation of VNN1 by saturated FFA was through a PPARα-mediated mechanism, HepG2 were treated with PPARα siRNA and incubated with PA for 24 h. Silencing of PPARα and treatment with PA did not affect VNN1 expression in HepG2 (FIGS. 17D and 17E).

These results show that MP-mediated angiogenesis occurs in a VNN1-dependent internalization into target cells.

Circulating MPs are Released During Experimental NASH, Express VNN1 and have Proangiogenic Effects

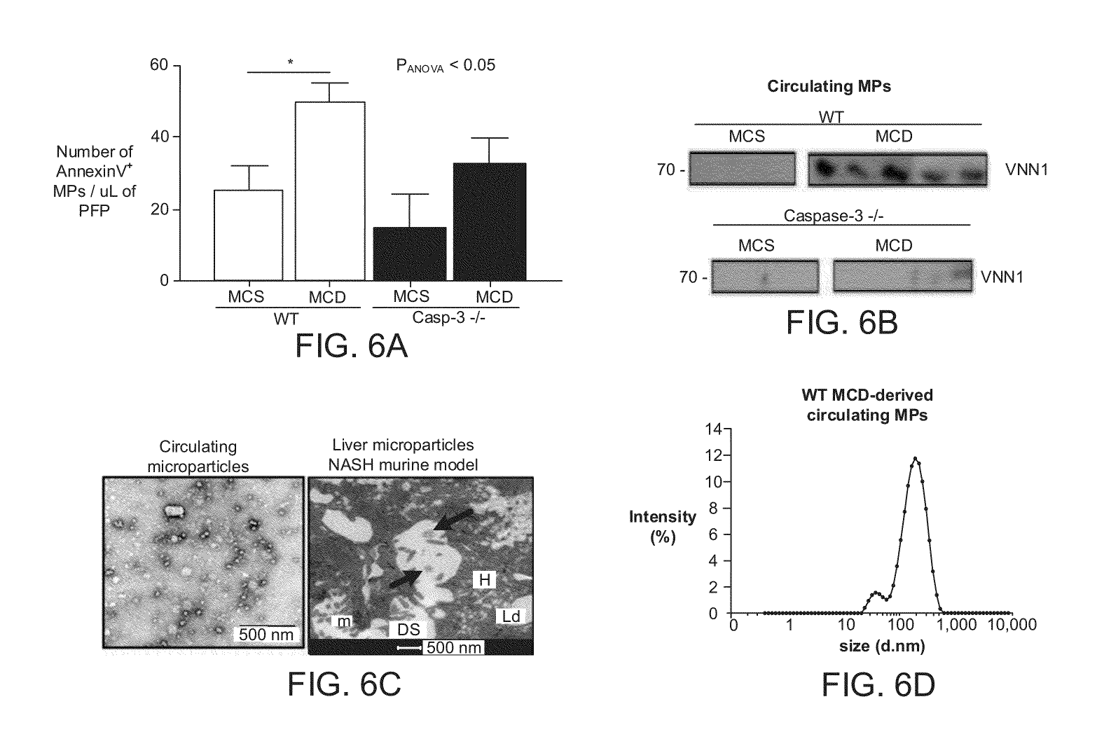

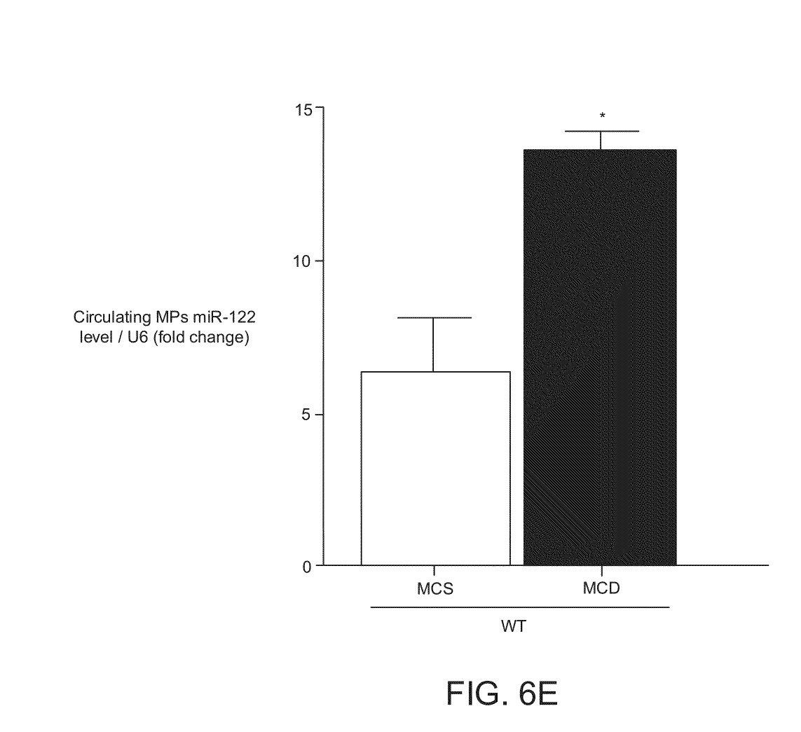

To further gain insight into the potential role of hepatocyte-derived MPs in hepatic angiogenesis during steatohepatitis development in vivo, a common dietary murine model of NASH was used. C57BL/6 mice were placed on a methionine and choline deficient (MCD) diet, which has been extensively shown to result in steatosis associated with significant inflammation and progressive fibrosis pathologically similar to human severe steatohepatitis. Indeed, after six weeks on this diet, mice developed steatohepatitis that histopathologically resembles human NASH. Strikingly, these changes were associated with a marked increase in circulating Annexin V positive MPs compared to mice receiving the control diet (MCS) and this increase resulted partially reduced in caspase 3 knockout mice (FIG. 6(A)), suggesting that caspase 3 activity may play an important role also in production and/or release of circulating MPs during experimental NASH. Furthermore, it was observed that only the circulating MPs isolated from WT MCD-fed mice expressed VNN1, compared to MP either isolated from mice which received the control diet (MCS) or caspase 3 KO mice (FIG. 6(B)).

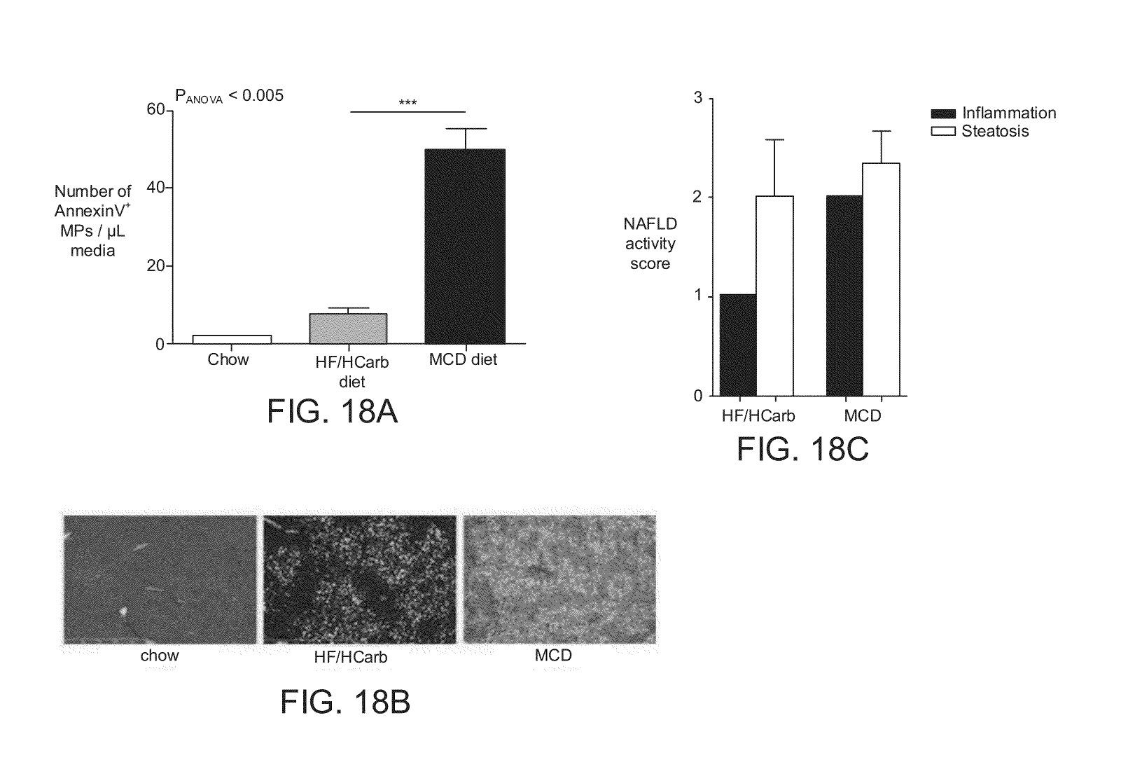

To establish whether the circulating levels of MPs correlate with the severity of liver pathology, the number of Annexin V positive MPs in blood of MCD fed mice was compared to those from animals fed a high fat, high carbohydrate diet (HF/HCarb) that results in hepatic steatosis in the absence of liver injury or inflammation [36, 37]. Indeed, a significant association between blood MPs and severity of liver histopathology was found (FIGS. 18(A)-18(C)). Moreover, light scattering and electron microscopy analysis demonstrated that the size range and morphology of blood MPs present in MCD-treated animals were similar to those found in supernatants of palmitic acid treated hepatocytes (FIGS. 6(C) and 6(D)). Finally, electron microscopy of liver sections from MCD-treated but not MCS-treated mice showed the presence of abundant microvesicles with characteristic morphology of MPs. These vesicles were present predominantly in perisinusoidal areas or space of Disse, the location in the liver between hepatocytes and a sinusoid. The MPs could be easily distinguished from hepatocyte microvilli that typically extend into this space (FIG. 6(C)). To determine the hepatocyte origin of the circulating MPs isolated from MCD- or MCS-fed mice, the expression of the hepatocyte-specific miR-122 [38, 39] was measured in the circulating MPs. Results show that miR-122 expression is markedly up-regulated in the circulating MPs isolated from MCD-fed mice comparing to the MPs isolated from mice that received the control diet (FIG. 6(E)).

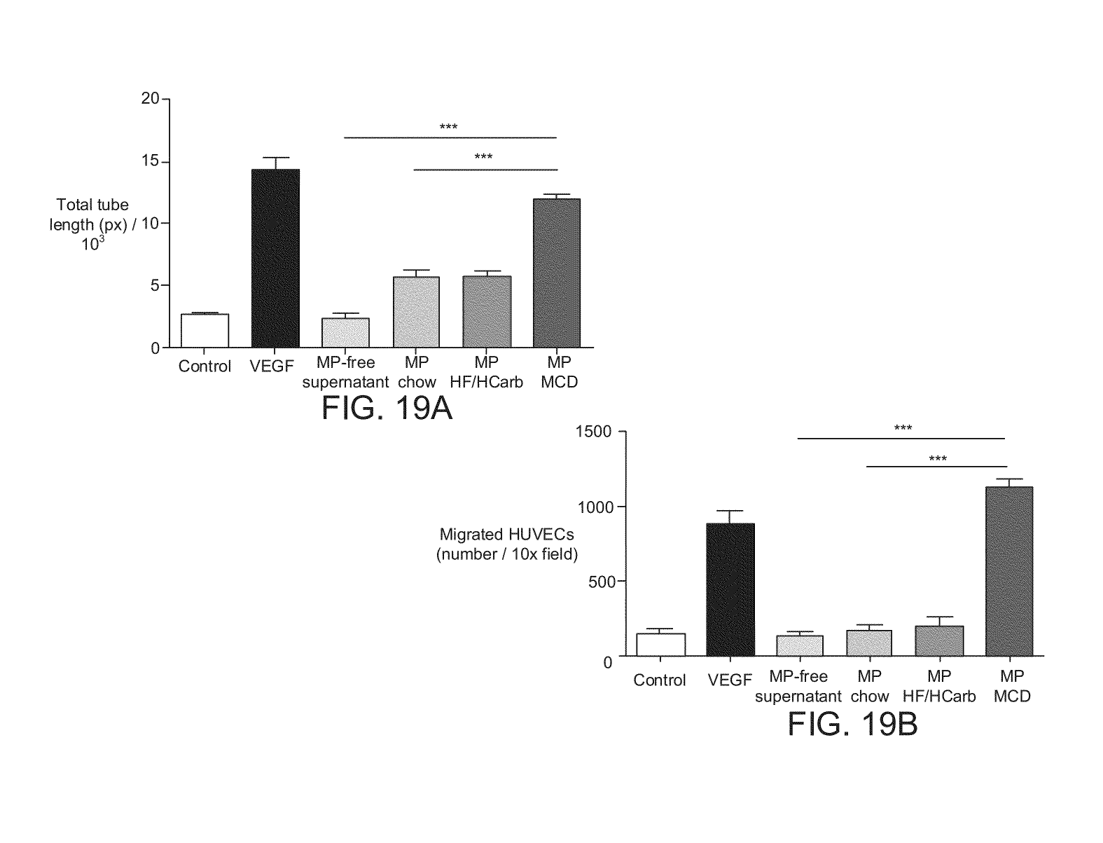

To determine whether circulating VNN1 positive MPs present in MCD-treated mice could have similar biological effects on endothelial cells as MPs derived from hepatocytes in vitro, additional experiments were performed where C57BL/6 mice were placed on MCD, high fat/high carbohydrates (HF/HCarb) or normal chow diets for 6 weeks for isolation of blood MPs. Platelet-free plasma was then harvested from the two groups of mice and MPs isolated. Endothelial cells were then treated with isolated MPs or MPs-free plasma and analyzed for tube formation and oriented migration. Blood MPs from MCD-treated mice induced marked angiogenic effects ex vivo to a similar extent to those found with MPs from hepatocytes treated with palmitic acid (FIGS. 19A and 19B).

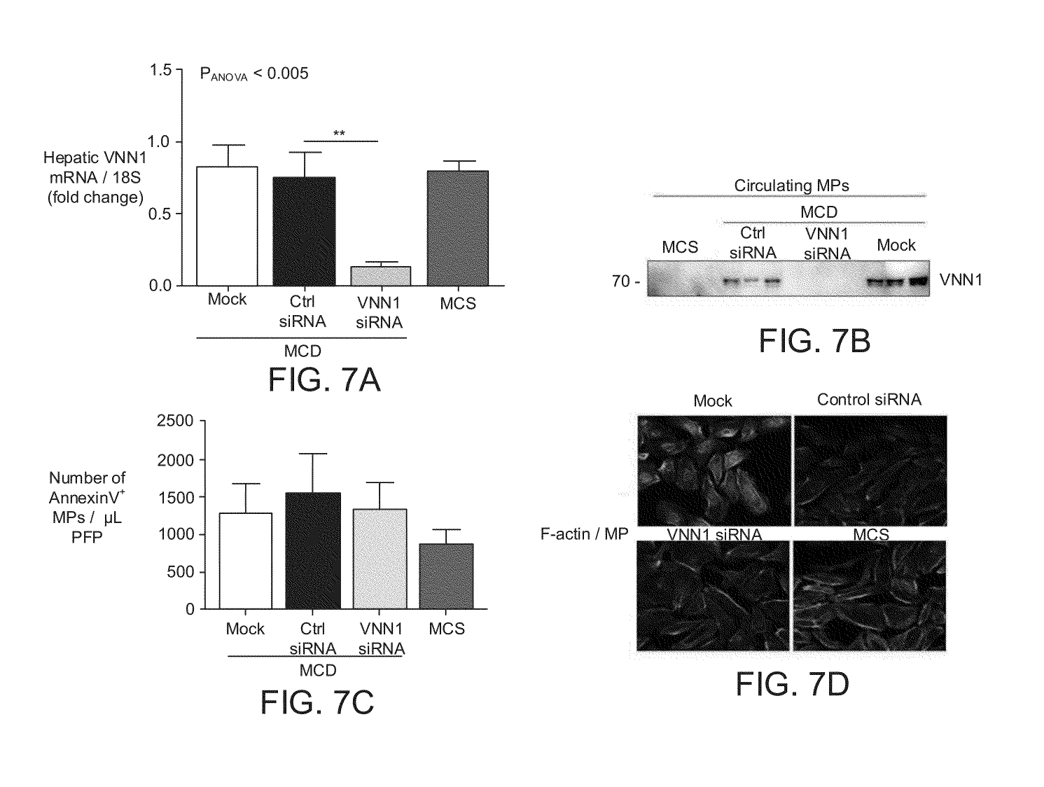

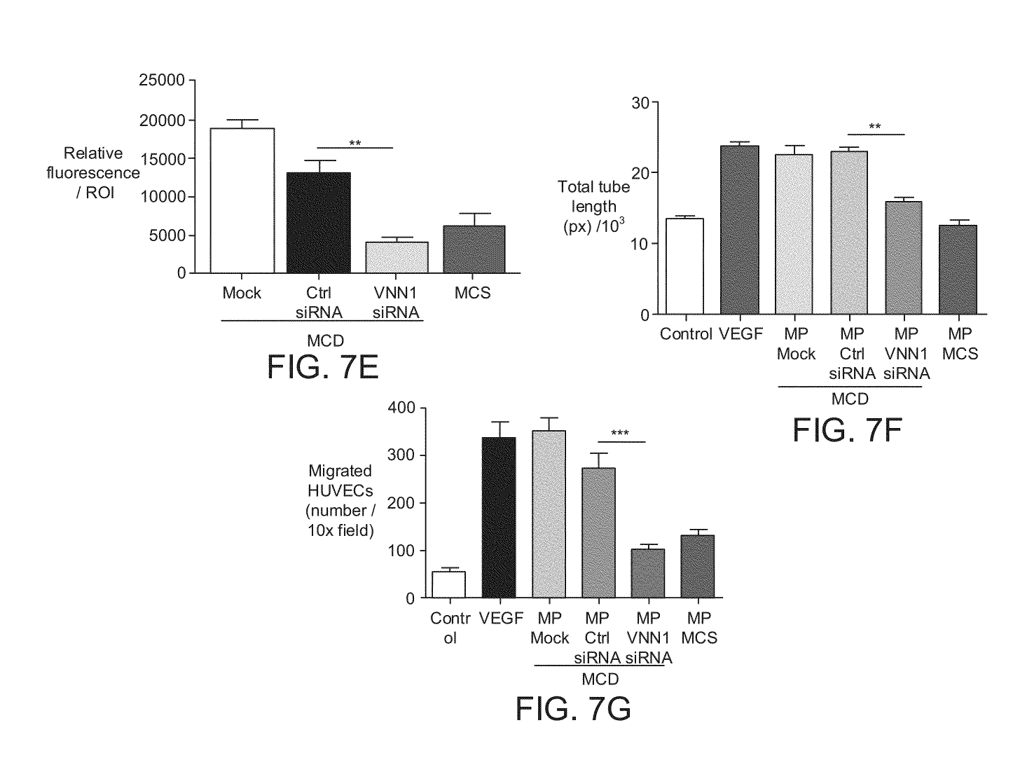

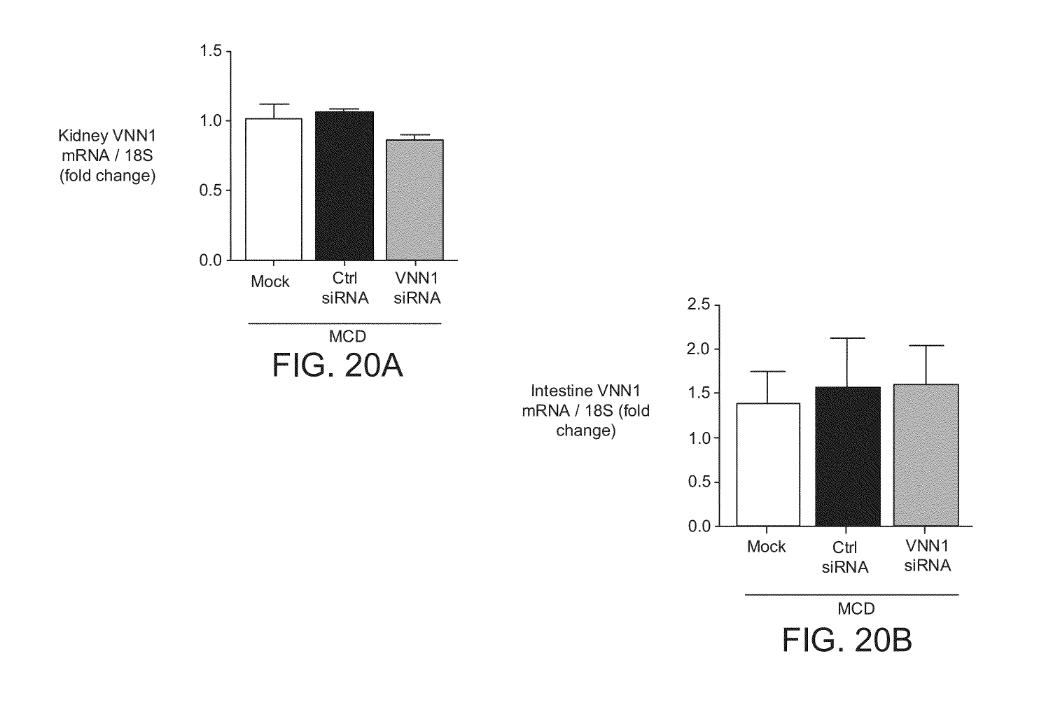

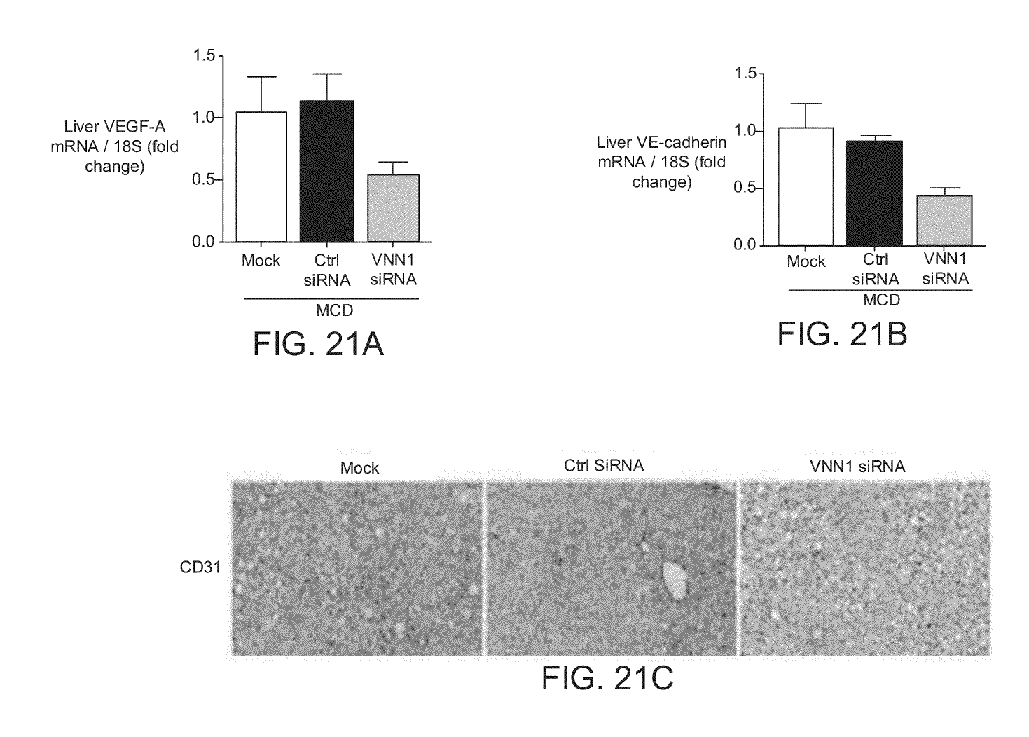

To evaluate the role of VNN1 in circulating MPs, C57BL/6 mice were placed on the MCD or control diet (MCS) for 6 weeks. MCDfed mice were further treated, weekly, with VNN1 siRNA or control siRNA (Ctrl siRNA) formulated with Invivofectamine Rx. Mice in the control PBS-treated group (Mock) were injected with PBS via the tail vein. Silencing RNA achieved significant inhibition of hepatic VNN1 of up to 90% comparing to PBS-treated mice (FIG. 7(A)), whereas VNN1 mRNA expression remained unchanged in two VNN1 expressing organs: kidney and intestine (FIGS. 20(A)-20(C)). Western blotting analysis of VNN1 level in circulating MPs isolated from each animal group confirmed that only MPs isolated from MCD-fed mice and not that isolated from MCS-fed mice, express VNN1. Treatment with VNN1 siRNA generated circulating MPs lacking of VNN1, compared to circulating MPs isolated from mock or control siRNA-treated (Ctrl siRNA) mice where VNN1 was still expressed (FIG. 7B). Importantly, the genetic knockdown of VNN1 did not influence the level of circulating MPs (FIG. 7(C)). Notably, internalization of circulating MPs lacking of VNN1 and more importantly, the ex vivo pro-angiogenic effects of these MPs on endothelial cells tube formation and chemotaxis, resulted drastically reduced compared to that induced by MPs isolated from either mock or control siRNA-treated mice (FIGS. 7D-7G). Additionally, mice placed on the MCD diet for 6 weeks and treated with VNN1 siRNA showed not only a reduction of mRNA expression of pro-angiogenic transcripts for VEGF-A and VE-cadherin but also of the histological marker for neovessels formation CD31, compared to the mice treated with control siRNA or PBS (mock) (FIGS. 21(A)-21(C)).

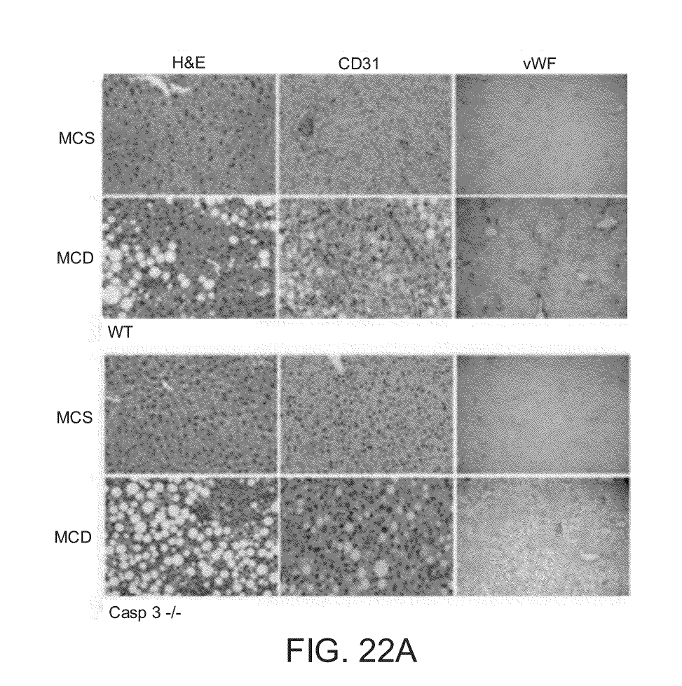

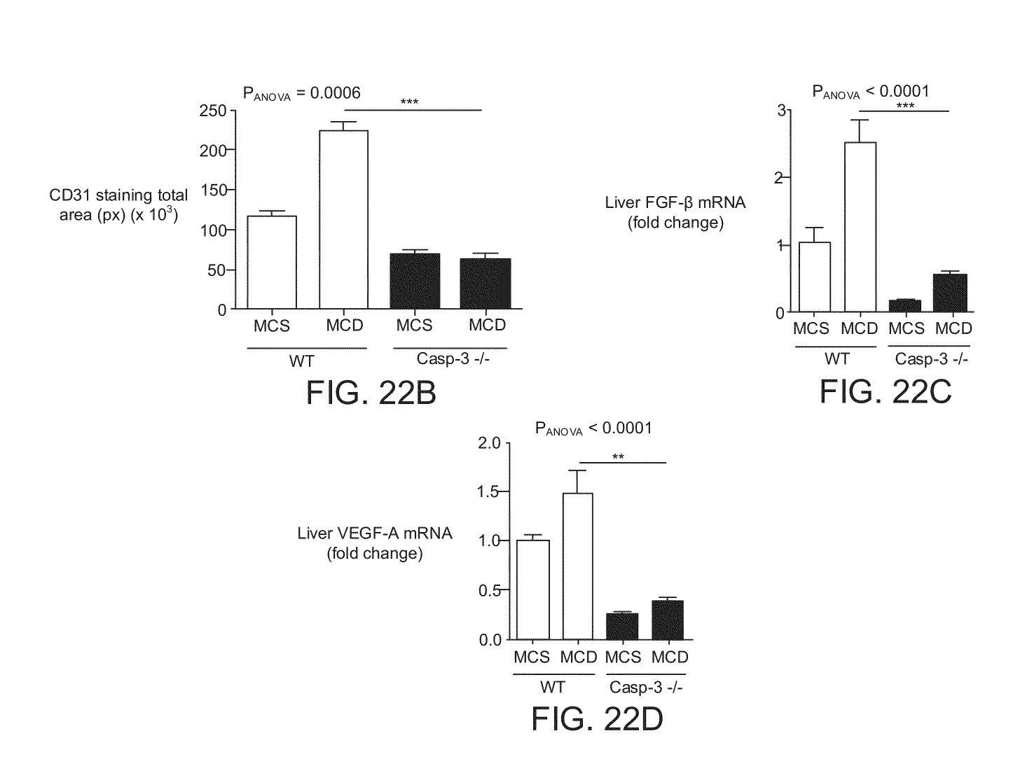

To further examine the potential role of MPs in hepatic pathological angiogenesis during NASH development, a variety of in vivo assays were performed by using liver specimens harvested from MCD- or MCS-fed C57BL/6 wild-type and caspase 3 KO (Casp3_{-/-}) mice. Histological examination showed that both Casp3_{-/-} and WT mice on the MCD diet developed predominantly macro-vesicular steatosis and lobular inflammation (FIG. 22(A)). In contrast, Casp3_{-/-} mice showed a significant reduction in pathological angiogenesis (FIGS. 22A, 22B and 22D) induced during the MCD diet (FIGS. 22A, 22C and 22D). Collectively, these findings identify hepatocyte-derived MPs as a potential novel link between lipotoxicity and angiogenesis with a crucial involvement of caspase 3 in this process.

### DISCUSSION

The main findings of this example relate to the mechanisms linking lipotoxicity in hepatocytes to angiogenesis. The findings demonstrate that overloading of hepatocytes with saturated lipotoxic free fatty acids (FFAs) results in release of pro-angiogenic factors in a process that requires caspase 3 activation. Hepatocyte-derived MPs were further identified as the putative pro-angiogenic factor both in vitro and in vivo. Comprehensive proteomic analysis further identify VNN1, an epithelial ectoenzyme involved in cell adhesion and migration, as one of the most highly expressed surface proteins in hepatocyte-derived MPs and functional studies demonstrate that the novel proangiogenic effects of MPs require internalization by endothelial cells in a process dependent on VNN1 expression and the lipid raft machinery.

Growing evidence suggests that angiogenesis plays a central role in chronic liver disease [7]. Particular attention has been focused on the potential role of formation of new blood vessels in the progression from hepatic steatosis, a generally benign condition characterized by over-accumulation of lipids in the liver to NASH which is, in turn, a potentially more severe condition associated with lipid overloading of the liver, inflammation, and a variable degree of fibrosis. Indeed, marked hepatic neovascularization has been reported in both patients with NASH as well as in experimental models of the disease and described to parallel the extent of detectable fibrosis [8-11, 40]. The pathogenic mechanisms resulting in angiogenesis in NASH remain poorly understood. Increased production and release of certain pro-angiogenic factors such as VEGF-A, likely as a result of local hypoxia due to enlarged hepatocytes (i.e. swollen with accumulated lipids) or VEGF-R2 signaling pathway, have been implicated [7, 41]. More recently, the degree of angiogenesis in the livers of patients with NASH has been shown to tightly correlate with the activation of caspase 3 in hepatocytes [14]. However, the molecular signaling events linking lipid overloading of hepatocytes and lipotoxicity to angiogenesis and liver damage remains completely unknown. Lipid overloaded hepatocytes may release pro-angiogenic signals that regulate endothelial cell migration and angiogenesis.

Using a variety of functional in vivo, ex vivo and in vitro assays, the studies in this example demonstrate that lipotoxicity induced by incubation of hepatocytes with saturated FFAs such as palmitic acid results in the release of proangiogenic factors that induce endothelial cell migration and vascular tube formation. While the contribution of inflammatory cells in angiogenesis during chronic liver damage has been extensively studied and characterized [42, 43], far less is known regarding the potential contribution of hepatocytes to this process. In the current studies, through several lines of evidence, hepatocyte-derived MPs, small membrane-bound particles released in a highly regulated manner from dying or activated cells, were demonstrated as a novel link between hepatocyte lipotoxicity and angiogenesis. MPs are a type of microvesicle that are released from the surface membranes of many cell types spontaneously or upon a variety of stressors [44, 45]. Recent studies have demonstrated an increase in circulating MPs in animal models of biliary cirrhosis and human cirrhosis and NASH [46-48]. An increasing body of evidence indicates that these MPs play a pivotal role in cell-to-cell communication [42, 44]. The function of MPs is dependent on the cell type from which they originate and their content.

Incubation of hepatocytes with palmitic acid resulted in a marked formation and release of MPs. The release of MPs from hepatocytes was dependent on caspase 3 activity suggesting that caspase activation is a critical event in the formation of these vesicles. A comprehensive analysis of the composition of the MPs using proteomic approaches has identified a number of potential novel target proteins that are unique for hepatocyte-derived MPs [49-51]. There were also several proteins that were previously shown to be present in MPs from other cell origins as well as in exosomes secreted by hepatocytes [40, 43, 49, 52]. Studies to systematically compare the protein composition of hepatocyte-derived MPs from MPs from other cell types, as well as from exosomes secreted by hepatocytes from various species could also be done.

The instant studies identified VNN1, a novel ectoenzyme anchored at the surface of epithelial cells that is highly expressed in hepatocytes, kidney and intestine and recently linked to important roles in cell adherence and migration as well as mediator of tissue inflammation in murine models of colitis [22]. The findings of large number of MPs inside tubular structures in the tube formation assays suggest that MPs are internalized by endothelial cells. Indeed, through a variety of functional studies, it was demonstrated that MPs exert their pro-angiogenic effects via internalization and this process depends on VNN1 expression on the surface of these vesicles and lipid rafts on target cells. Incubation of endothelial cells with hepatocyte-derived MPs with genetically knockdown VNN1 expression or in the presence of VNN1 neutralizing antibody prevented MPs endocytosis and abrogated the effects of MPs on angiogenesis. The in vitro observations are further extrapolated to in vivo conditions since it was found that MPs from palmitic acid treated hepatocytes induced marked angiogenesis in Matrigel plugs in athymic BALB/c nude mice.

Moreover, the instant studies demonstrate that a large number of MPs were present in the livers and in circulation of mice fed with the MCD diet, one of the most common animal models of NASH [53] and that a significant amount of these circulating MPs originated from hepatocytes. While only MPs from these mice but not control animals expressed VNN1 and were capable of inducing angiogenesis ex vivo. Silencing of VNN1 in vivo during the experimental NASH resulted in selective knockdown of VNN1 expression in liver and circulating MPs, with a consequent dramatic decrease of internalization as well as pro-angiogenic effects of MPs, but not of their amount in the blood stream. Moreover, MCD-fed animals showed marked increase of pathological angiogenesis in the liver, which resulted reduced in VNN1 siRNA-treated animals.

Similarly, in accordance with the in vitro and ex vivo data, a decrease in circulating VNN1 expressing MPs by suppression of caspase 3 activation in Casp3_{-/-} mice resulted in protection against hepatic angiogenesis induced by the MCD diet. The localization of MPs in the space of Disse, the location in the liver between hepatocytes and a sinusoid is highly suggestive that these microvesicles act in a paracrine manner. While VNN1 was focused on in the instant studies as an important mediator of MPs effects on target cells as well as a biomarker to monitor MPs in blood, other protein targets, lipids as well as the RNA including microRNAs could have additional or synergistic effects to that found for VNN1.

In summary, the data in the present example support a model in which during lipotoxicity, hepatocytes exposed to saturated lipotoxic free fatty acids like palmitic acid, release MPs in a process requiring caspase 3 activation that, in turn, can initiate endothelial cell migration, and angiogenesis via VNN1 mediated MP internalization. Taken together, these results identify hepatocyte-derived VNN1 expressing MPs as an attractive potential target for developing novel anti-angiogenic therapeutic strategies for the treatment of NASH as well as novel circulating biomarkers of liver damage.

### Materials and Methods Animal Studies

C57BL/6 caspase 3 knock out (casp3_{-/-}) mice, were generously provided by Dr. Mina Woo (University of Toronto). These mice were generated by deleting exon 3 of the CPP32/caspase 3 gene as previously described in detail [54]. They appear healthy and do not have a particular acute phenotype; however, they show a slight decrease of life span. Casp3_{-/-} and wild-type littermates, 20 to 25 gm of body weight, 7 weeks old, were placed on a methionine and choline-deficient (MCD) diet (MP biomedicals, Solon, Ohio), which has been extensively shown to result in steatosis associated with significant inflammation and progressive fibrosis, pathologically similar to human severe steatohepatitis (NASH) [28, 55]. A methionine and choline-supplemented (MCS) diet (MP biomedicals, Solon, Ohio) and chow diet were used as control diet. A Western diet with high fat and high carbohydrates (HF/HCarb) content has been used for 6 weeks as a model of diet-induced nonalcoholic fatty liver disease (NAFLD). Total body weight was measured weekly in all mice. Mice were sacrificed after 6 weeks on their respective diets and the liver and blood were collected under deep anesthesia as previously detailed (34). Athymic BALB/c nude mice (Case Western Reserve University athymic nude mice facility, Cleveland, Ohio, USA), 6 weeks-old, 20 to 25 gm of body weight were used for the in vivo Matrigel migration and angiogenesis assay. All animals were treated in compliance with the Guide for the Care and Use of Laboratory Animals (National Academy of Science, Washington, D.C.) and animal procedures were approved by the University of California, San Diego Institutional Animal Care and Use Committee.

### Histopathology and Immunohistochemistry

Liver tissue was fixed in 10% formalin and paraffin embedded. Tissue sections (5 μm) were prepared, stained for hematoxylin and eosin (H&E) to assess histological changes including degree of steatosis, ballooning of hepatocytes, and inflammation under light microscopy. The presence of tissue neovessel formation was assessed by immunostaining for polyclonal rabbit antibody anti-CD-31 (1:25; Abcam, Cambridge, Mass.) and polyclonal rabbit antibody anti-vonWillebrand factor (1:300; Dako, Carpinteria, Calif., USA). De-paraffinized sections were immersed in 3% H₂O₂ in water for 15 minutes to eliminate the endogenous peroxidase activity. Sections were processed for heat-induced epitope retrieval for 20 minutes by using Dako Target Retrieval Solution pH 6.0 (Dako, Glostrup, Denmark) and stained overnight at 4° C. After the incubation with the secondary antibody, immune complexes were detected by using DakoEnVision with HRP system (Dako, Glostrup, Denmark), according to the manufacturer's instructions. The quantification of CD31 staining was performed by customized histology quantification software provided by Wimasis (Munich, Germany) and CD31% of staining was reported in the graph.

### Cell Culture

Primary rat hepatocytes were purchased from Becton & Dickinson (BD, Franklin Lakes, N.J., USA) and maintained in Hepato-STIM hepatocytes defined medium (BD, Franklin Lakes, N.J., USA), supplemented with 5 μg of EGF and 2 mM of L-glutamine. Human hepatoma cell line (HepG2) was maintained in Dulbecco's Modified Eagle's medium (DMEM) (Life Technologies, Grand Island, N.Y., USA), supplemented with 10% fetal bovine serum (CellGro, Manassas, USA), 5,000 U/mL penicillin and 5,000 μg/mL streptomycin sulfate in 0.85% NaCl. Human umbilical vein endothelial (HUVEC) cells were maintained in EGM-2 growth medium (Lonza, Basel, Switzerland), supplemented with several angiogenic and growth factors (SingleQuots, Lonza, Basel, Switzerland), according to the instructions of the manufacturer. Cells were cultured at 37° C. in a 5% CO₂ humidified environment and used between passage 2 and 6. For treatments, HUVECs were incubated with growth factor-free media EBM-2 (Lonza, Basel, Switzerland). Long-chain free fatty acids (FFAs), palmitic, stearic, oleic and linoleic acid (Sigma-Aldrich, St. Louis, Mo., USA) were dissolved in 95% ethanol (stock solution 100 mM) and stored at −20° C. before the experiments.

### Microparticles Isolation and Purification

For microparticles (MPs) isolation, HepG2 and primary rat hepatocytes were seeded onto a 100 mm dish and cultured until reaching 80-85% of confluence. Cells were incubated with 0.25 mM of palmitic, stearic, oleic or linoleic acid (FFA) in serum-free DMEM, supplemented with 1.1% penicillin and streptomycin, 1% endotoxin-free bovine serum albumin (BSA), for up to 24 h. The amount of FFAs used is physiologically relevant and has been extensively shown being consistently comparable to the levels detected in obese patients [15]. The uptake of FFAs from hepatocytes was evaluated by Oil red-O staining kit (Cayman, Ann Arbor, Mich., USA) according to the manufacturer instruction. To determine the effect of caspase 3 activity, hepatocytes were treated with a selective caspase 3 inhibitor, Ac-DEVD-CHO (BD Pharmingen, Franklin Lakes, N.J., US). Control cells were incubated with the same serum-free media in association to the vehicle that was used to dissolve the FFAs. After 24 h, the supernatant were collected and centrifuged twice at 3,000 rpm for 15 minutes to remove cell debris and aggregates. The supernatant was then transferred to new tubes and ultracentrifuged at 100,000 g for 90 minutes at 10° C., to avoid contamination of exosomes [56].

The supernatant was collected in new tubes and used as a MP-free control and the pelleted MPs were resuspended in 200 μl of PBS for flow cytometry or in 500 μl of EGM-2 growth medium for subsequent in vitro studies. The concentration of microparticles μg/mL was determined by the BCA protein assay and different concentrations of MPs (50, 125, 250 and 500 μg/mL) were used to perform the dose-dependent angiogenesis in vitro assays. For selected purposes, in particular for the characterization analyses, crude MPs were purified by 10-70% sucrose gradient ultracentrifugation at 150,000 g for 18 h at 10° C., collected as fractions in new tubes. Fractions were resolved by a SDS-PAGE precast polyacrylamide gels electrophoresis (Biorad, Hercules, Calif., USA), which were then stained by Silver staining (Invitrogen, Grand Island, N.Y., USA) to detect proteins in each fraction. Fractions with a density between 1.17 and 1.25 mg/mL and corresponding to the microparticles, were combined, resuspended in PBS and ultracentrifuged at 100,000 g for 1 h at 10° C. to remove sucrose residues. For the isolation of MPs from caspase 3_{-/-} and wild-type mice, blood was collected by cardiac puncture into BD vacutainer tubes containing 100 μl of acid citrate dextrose (ACD; 0.085 M sodium citrate, 0.0702 M citric acid, 0.111 M dextrose, pH 4.5) as anticoagulant (Medcompare, South San Francisco, Calif., USA). Whole blood was centrifuged for 15 minutes at 200 g at room temperature to get the platelet rich plasma (supernatant). Platelet rich plasma was centrifuged again for 2 minutes at 13,000 g at room temperature to remove contaminating platelets and obtain platelet-free plasma (PFP). MPs were incubated in the dark for 30 minutes at room temperature with or without 5 μl of Alexa® Fluor 488-conjugated Annexin V (Molecular Probe, Eugene, Oreg.). A small aliquot of MPs was used to determine the MPs diameter by using Dynamic Light Scattering Zetasizer (Malvern, Worcestershire, UK).

### Flow Cytometry

Flow cytometry analyses for microparticles (or endothelial cells) detection were performed by using BD LSRII Flow Cytometer System (BD Biosciences, San Jose, Calif., USA) and the data were analyzed using FlowJo software (TreeStar Inc., Ashland, Oreg., USA). For microparticles analysis, a standardization was achieved by using 1 μm latex fluorescent beads (Sigma-Aldrich, St Louis, Mo., USA) and ultraviolet 2.5 μm flow cytometry alignment beads (Invitrogen, Grand Island, N.Y., USA).

Forward scatter (FS) and side scatter (SS) limits were plotted on logarithmic scales to best cover a wide size range. Single staining controls were used to check fluorescence compensation settings and to set up positive regions. For endothelial cells analysis, cells were incubated with labeled microparticles for 16 h, tripsinized and resuspended in fresh medium. Cells were filtered before FACS to eliminate clumped cells.

### Tube Formation

HUVECs (0.5-1×10⁵ cells per well) were seeded onto a coated 24-well plate culture dishes with 200 μl/cm₂ (thick gel method) of Matrigel (BD, Franklin Lakes, N.J., USA) and cultured in EBM-2 basal medium supplemented with 1.1% of streptomycin and penicillin in the presence of 100 ng/mL of VEGF (Peprotech, Rocky Hill, N.J., USA), hepatocytes-derived MPs, MP-free supernatant or conditioned supernatant collected after 24 hours of treatment with FFAs. In selective studies, a specific inhibitor for RhoA-ROCK (Y27632, 10 μM) was used in association with MPs in the treatment of endothelial cells. For the ex vivo studies, PFP isolated from different groups of mice (chow, HF/HCarb, MCD fed mice and mice injected with control RNA, VNN1 siRNA or PBS as vehicle) were ultracentrifuged at 100,000 g for 1 h at 10° C. to pellet the blood MPs. HUVECs were treated with MPs (or blood pure MPs) and incubated for 5 h at 37° C. Tube formation was investigated by using an inverted microscope at 4× magnification and images were captured. Tube formation image analysis was assessed by using Wimtube software (Wimasis, Munich, Germany) and the values referred to total tube length (pixel) were used for statistical and data analysis.

### Cell Migration

In vitro migration studies have been performed by using human umbilical cell endothelial cells (HUVECs). Wound healing assay was performed to analyze chemokinesis (non-oriented migration) by using collagen-coated 6-well culture plate, where a silicon elastomer (PDMS) strip was placed vertically to the bottom of every well to create the artificial wound. HUVECs were plated and grown until reaching the complete confluence. HUVECs were treated with EBM-2 supplemented with 100 ng/mL of VEGF (Peprotech, Rocky Hill, N.J., USA), hepatocytes-derived MPs, MP-free supernatant or supernatant collected after 24 hours of treatment with FFAs. The strips were removed carefully and migration of cells was detected by a confocal microscope, capturing one picture per field every 10 minutes for 48 hours. Mitomycin (1 μg/ml) was used to inhibit cell proliferation during wound healing assay. Data were analyzed by using Wimscratch software (Wimasis, Munich, Germany) in order to get the total cell-covered area (pixel). Boyden's chamber assay was performed to analyze chemotaxis (oriented migration) by using cell culture inserts 8 μm pore size (Millipore, Billerica, Mass., USA) and 24-well plate. Each well was filled with 500 μl of serum free EBM-2 media in presence or absence of the following chemoattractants: 100 ng/mL VEGF; hepatocytes-derived MPs; MP-free supernatant or conditioned supernatant. The inserts were placed on top of each well and 150 μl of cell suspension (5×104 cells) was added. Plates were incubated overnight at 37° C., and then the filters were removed and stained with Vectashield mounting medium with 4′,6-diamino-2-phenylindole (DAPI) (Vector Labs, Burlingame, Calif., USA).

Migrated cells were detected with a fluorescence microscope and number per field of their nuclei was counted. For the ex vivo studies, PFP isolated from different groups of mice (chow, HF/HCarb, MCD fed mice and mice injected with control siRNA, VNN1 siRNA or PBS as vehicle) were ultracentrifuged at 100,000 g for 1 h at 10° C. to pellet the blood MPs.

### In Vivo Matrigel Migration and Angiogenesis Assay

To screen the capability of hepatocyte-derived MPs for induction of angiogenesis, athymic BALB/c nude mice were subcutaneously injected with 0.5-1 mL ice-cold Matrigel Matrix Growth Factors Reduced (BD, Franklin Lakes, N.J., USA), mixed with 100 ng/mL VEGF, hepatocytes-derived MPs or MP-free supernatant. A volume of Matrigel only was used as negative control. After 14 days, Matrigel plugs were removed along with tissue around for orientation, fixed in 10% formalin and embedded in paraffin. Sections (4 μm) were successively stained for Masson's Trichrome to detect endothelial cells (in dark red). Number of cells migrated into the plug following the treatment, was evaluated. The number of cells migrated was reported in the graph as number per field.

### Proliferation Assay

Fat-laden HepG2-derived microparticles were treated with 4 μg/mL of neutralizing rabbit antibody anti-VNN1 (Epitomics, Burlingame, Calif., USA) for 30 minutes. Serum-starved HUVECs were treated with hepatocytes-derived microparticles, MP-free supernatant, VNN1-neutralized microparticles, 100 ng/mL of VEGF and negative control for up to 16 h. Cells were then treated with the 5-bromo-2′-deoxyuridine (BrdU) up to 4 h. After removing the labeling medium, cells were resuspended in anti-BrdU antibody (1:8, BD) for 30 minutes at room temperature. A goat antimouse IgG Alexa-Fluor 488 (1:500, Life Technologies) was added and cells were incubated for 30 minutes at room temperature. Cells were counterstained with propidium iodide. FITC-positive cells were detected by BD LSRII Flow Cytometer System and quantified as described previously [57].

In Vitro and In Vivo siRNA-Mediated Knockdowns

HepG2 were trypsinized and 8×10⁵ cells were seeded in a 6-well plate. To knockdown VNN1 or PPARα in hepatocytes, HepG2 were incubated with 50 nM of silencing RNA (siRNA) for VNN1 or PPARα and control siRNA (Ctrl siRNA) dissolved in Lipofectamine 2000 (Life Technologies, Carlsbad, Calif., USA). Transfection was performed according to the manufacturer instructions. After 48 h post transfection, cells were treated with 0.25 mM of palmitic acid for 24 h and microparticles were isolated from the supernatant by ultracentrifugation. The efficiency of transfection has been confirmed in hepatocytes and microparticles by Western blotting as described in the “Protein analysis” paragraph. Additionally, a flow cytometry analysis of hepatocytes-derived microparticles was performed by using a PE-conjugated anti-VNN1 antibody (Santa Cruz, Calif., USA) to test the efficiency of the transfection. To knockdown cdc42, HUVECs were trypsinized, seeded in a 6-well plate and incubated with 50 nM of the small interfering RNA (siRNA) for cdc42 and control siRNA (Ctrl siRNA) dissolved in Lipofectamine 2000 (Life Technologies). Transfection was performed according to the manufacturer specific protocol. After 48 h post transfection, cells were used for chemotaxis and tube formation assays as detailed in the “tube formation” and “migration studies” paragraphs. In vivo VNN1 knockdown experiments during experimental NASH were assessed by weekly injection of VNN1 stealthRNA (Life Technology), control siRNA (Ctrl siRNA) or PBS as vehicle (mock) in C57BL/6 mice fed with the MCD diet for 6 weeks. To deliver siRNA, we used Invivofectamine Rx (Life Technology) according to the manufacturer's instructions. For the siRNA injection the following approach was used: week 1, 3 and 5 mice received 1.5 mg/Kg of siRNA Invivofectamine Rx or controls whereas week 2, 4 and 6 mice received 0.75 mg/Kg of siRNA Invivofectamine Rx or controls. As previously described, the siRNA injection in vivo does not cause any major liver pathological consequences or side effects in mice [58].

Internalization of Microparticles into Endothelial Cells

Internalization of hepatocyte-derived MPs into HUVECs and the role of VNN1 and lipid raft were evaluated by flow cytometry, indirect immunofluorescence and electron microscopy. To investigate the MPs internalization into HUVECs, by flow cytometry, MPs were stained with 1 μM of Calcein, AM (MPCalcein) (BD Biosciences), a fluorescent dye with an emission wavelength of 515 nm, for 1 h in the dark at 37° C. MPs were centrifuged twice with PBS for 30 minutes at 35,000 rpm and resuspended in 500 μL of EGM medium. To block MPs internalization, MPsCalcein were incubated with or without 4 μg/mL of rabbit polyclonal anti-VNN1 antibody (S3055; Epitomics, Burlingame, Calif., USA) for 30 minutes at RT. A rabbit anti-GAPDH antibody (Abcam, Cambridge, Mass., USA) was used as a control. After the incubation, MPs were incubated with serum-starved HUVECs seeded in a 24-well plate (15×104 cells per well) for 6 h at 37° C. Cells were trypsinized and FITC positive HUVECs were detected by BD LSRII Flow Cytometer System (BD) and counted. For the immunofluorescence studies, HUVECs were seeded in 4-chamber culture slides (BD Biosciences, San Jose, Calif., USA) and serum starved for 4 h. MPs were isolated from the supernatant of HepG2 treated with palmitic acid, as reported above, resuspended in 1 mL of Diluent C (G8278, Sigma-Aldrich, St Louis, Mo., USA) and gently pipetted to insure a complete dispersion. The 1 mL MPs suspension was added to 1 mL of Diluent C with 4 μL of PKH26 solution (MP_{PKH26}), a lipophilic dye that stably integrates into the cell membrane (MINI26, Sigma-Aldrich, St Louis, Mo., USA).

The MPs suspension was incubated in the dark for 4 minutes, and then 2 mL of 1% BSA was added and kept for 1 minute to stop the reaction. MPs suspension was then ultracentrifuged for 30 minutes at 35,000 rpm and pellet was resuspended in 200 μL of HUVECs medium. HUVECs seeded in the culture slide were incubated with MPsPKH26 for 1 and 6 h at 37° C. in the dark. After incubation, medium was removed from each well and HUVECs were washed twice with PBS and fixed with 4% paraformaldehyde solution in PBS for 10 minutes at room temperature. HUVECs were then stained with 5 μL of 488-Phalloidin (Invitrogen, Grand Island, N.Y., USA) in 200 μL of PBS according to the manufacturer instructions. After washing 3 times with PBS, HUVECs were stained with 4′,6-diamino-2-phenylindole (DAPI) (Vector Labs, Burlingame, Calif., USA). Slides were observed and images captured by using an Olympus FV1000 Spectral Confocal. The role of the lipid raft in the internalization of MPs into HUVECs was investigated by cholesterol depletion and inhibition of the lipid raft-associated protein caveolin-1. HUVECs (4×103 cell per well) were seeded onto a 24-well plate and depletion of cholesterol was assessed by a treatment with 10 mM of Methyl-β-cyclodextrin (MβCD) in serum-free EBM-2 medium for 15 minutes at 37° C., followed by wash with fresh medium. To inhibit caveolin-1 (cav-1), HUVECs seeded in a 24-well plate were treated with 2.5 μg/mL of rabbit anti-human caveolin-1 neutralizing antibody (cav-1 nAb) (Sigma Aldrich) in serum-free EBM-2 medium containing 0.01% of Triton X-100 for 30 min at 37° C., as previously described [27]. Cholesterol depleted or cav-1 nAb HUVECs were incubated for 6 h with MPPKH26 to assess the internalization by immunofluorescence.

The interaction between VNN1 on the MPs surface and the lipid raft on the HUVECs was assessed by electron microscopy, as previously reported [59]. After washing, 1-2 mL of cold 4% paraformaldehyde in 0.1 M phosphate buffer was pipetted onto the HUVECs and they were gently lifted off tissue culture plates with cell lifters. After a short low speed spin, fix was removed from pellets and fresh fix added to resuspend the cells. They were then fixed at 4° C. for one hour and washed extensively. After blocking for 30 minutes with 1% BSA in PBS, cells were incubated 0/N at 4° C. with the rabbit polyclonal antibody anti-human VNN1 (ID: 8876; Epitomics) gently rotating. Following washing, 15 nm protein A gold conjugate (1:25 in 1% BSA) (Ted Pella, Redding, Calif., USA) was bound to VNN1 for two hours at room temperature, gently rotating. The cells were washed carefully and HRP reacted with 10 mg/ml 3,3-Diaminobenzidine (DAB) in PBS for 5 minutes then with DAB in PBS with 0.0003% H₂O₂ for 30 minutes in the dark. After washing, cells were refixed with 1% glutaraldehyde and processed for routine electron microscopy. After standard ethanol dehydration, pellets were embedded in LX112 media (Ladd, Willston, Vt., USA) and polymerized for 48 hours at 60° C. Blocks were sectioned to 70 nm and grids viewed unstained to enhance HRP-DAB reaction.

### Electron and Confocal Microscopy

For transmission electron microscope (TEM), microparticles were adhered to 100 mesh Formvar and carbon coated grids for 5 minutes at room temperature. Grids were washed once with water, stained with 1% uranyl acetate (Ladd Research Industries, Williston Vt.) for 1 minute dried and viewed using a JEOL 1200 EXII transmission electron microscope. Images were captured using a Gatan Orius 600 digital camera (Gatan, Pleasanton, Calif.). Liver samples were collected from the MCD mice after a short liver perfusion with 10 mL of 4% paraformaldehyde in 0.15 M sodium cacodylate buffer, pH 7.4 by using a 21 G needle. Samples were immersed in modified Karnovsky's fixative (2.5% glutaraldehyde and 2% paraformaldehyde in 0.15 M sodium cacodylate buffer, pH 7.4) for at least 4 hours, post fixed in 1% osmium tetroxide in 0.15 M cacodylate buffer for 1 hour and stained en bloc in 3% uranyl acetate for 1 hour. Samples were dehydrated in ethanol, embedded in Durcupan epoxy resin (Sigma-Aldrich), sectioned at 50 to 60 nm on a Leica UCT ultramicrotome, and picked up on Formvar and carbon-coated copper grids. Sections were stained with 3% uranyl acetate for 5 minutes and Sato's lead stain for 1 minute. Grids were viewed using a JEOL 1200EX II (JEOL, Peabody, Mass.) transmission electron microscope and photographed using a Gatan digital camera (Gatan, Pleasanton, Calif.). Images of the MPs internalized by HUVECs were captures by an Olympus FV1000 Spectral Confocal by using 40× lens and Alexa-fluor 488 and Texas red filters. Analysis of images was performed by using Image J software (Version 1.4.3.67). For the immunogold-electron microscopy related to the internalization of VNN1-positive MPs through the lipid raft, refer to the paragraph entitled “internalization of microparticles into endothelial cells”.

RNA Isolation, Real-Time PCR and miRNA Analysis

Tissue (liver, intestine, spleen and kidney) from MCD- and MCS-fed mice (Casp-3−/−, WT, treated with VNN1 siRNA and controls) was homogenized using the FastPrep 24 bead homogenization system. Endothelial cells (HUVECs) or HepG2 were trypsinized, resuspended in lysis buffer and homogenized by mechanical fraction by using a 1 mL syringe. Total RNA was isolated using RNeasy kit (Qiagen, Valencia, Calif.) and reverse transcribed by iScript cDNA synthesis kit (Biorad, Hercules, Calif., USA) according to the manufacturer instructions. The concentration and purity of RNA was assessed by NanoDrop (Thermo Scientific). Quantitative Real time PCR was performed on a BioRad Cycler (BioRad) by using SYBRGreen real time PCR master mix (Kapabiosystem, Woburn, Mass., USA) according to the manufacturer instructions. The housekeeping gene 18S was used as an internal control.

For isolation and quantification of hepatocyte-specificmiR-122, PFP isolated from MCD-fed mice, as described in the “Microparticles isolation and purification” paragraph, was incubated with 10 μg/mL of RNase (Roche, Indianapolis, Ind., USA) for 30 min at 37° C. to remove any RNAs eventually stuck on the external leaflet of the circulating MPs. Circulating MPs were then ultracentrifuged at 100,000 g for 90 min at 10° C. Total encapsulated RNAs in MPs, including miRNAs, were isolated by miRNeasy Mini kit (QIAGEN). cDNA was synthesized using specific miRNA primers (Applied Biosystems) in TaqMan microRNA Reverse Transcription kit (Applied Biosystems). MiRNA expressions were detected using TaqMan probe (Applied Biosystems) on 7300 Real time PCR system (Applied Biosystems). The U6 snRNA was used as an internal control and to normalized miR-122 expression.

### Sample Preparation for MS

Protein samples were diluted in THE (50 mM Tris pH 8.0, 100 mM NaCl, 1 mM EDTA) buffer. RapiGest SF reagent (Waters Corp.) was added to the mix to a final concentration of 0.1% and samples were boiled for 5 min. TCEP (Tris(2-carboxyethyl) phosphine) was added to 1 mM (final concentration) and the samples were incubated at 37° C. for 30 min. Subsequently, the samples were carboxymethylated with 0.5 mg/ml of iodoacetamide for 30 min at 37° C. followed by neutralization with 2 mM TCEP (final concentration). Proteins samples prepared as above were digested with trypsin (trypsin:protein ratio—1:50) overnight at 37° C. RapiGest was degraded and removed by treating the samples with 250 mM HCl at 37° C. for 1 h followed by centrifugation at 14,000 rpm for 30 min at 4° C. The soluble fraction was then added to a new tube and the peptides were extracted and desalted using Aspire RP30 desalting columns (Thermo Scientific) [60].

### LC-MS-MS Analysis

Trypsin-digested peptides were analyzed by high pressure liquid chromatography (HPLC) coupled with tandem mass spectroscopy (LC-MS/MS) using nano-spray ionization[61]. The nano-spray ionization experiments were performed using a TripleT of 5600 hybrid mass spectrometer (ABSCIEX) interfaced with nano-scale reversed-phase HPLC (Tempo) using a 10 cm-100 micron ID glass capillary packed with 5-μm C18 Zorbax™ beads (Agilent Technologies, Santa Clara, Calif.). Peptides were eluted from the C18 column into the mass spectrometer using a linear gradient (5-60%) of ACN (Acetonitrile) at a flow rate of 250 μl/min for 1 h. The buffers used to create the ACN gradient were: Buffer A (98% H2O, 2% ACN, 0.2% formic acid, and 0.005% TFA) and Buffer B (100% ACN, 0.2% formic acid, and 0.005% TFA). MS/MS data were acquired in a data dependent manner in which the MS1 data was acquired for 250 ms at m/z of 400 to 1250 Da and the MS/MS data was acquired from m/z of 50 to 2,000 Da. For Independent data acquisition (IDA) parameters MS1-TOF 250 milliseconds, followed by 50 MS2 events of 25 milliseconds each. The IDA criteria, over 200 counts threshold, charge state+2-4 with 4 seconds exclusion. Finally, the collected data were analyzed using MASCOT® (Matrix Sciences) and Protein Pilot 4.0 (ABSCIEX) for peptide identifications.

### Protein Analysis

HepG2 were treated with 1% BSA, 0.25 mM of palmitic acid or oleic acid for 24 hours. Whole cell lysates were digested in 400 μL of RIPA buffer containing Protease Inhibitor Cocktail Tables (Roche). Microparticles were isolated from the same cells or from the animal blood, as described in the ‘MPs isolation and purification’ paragraph, and resuspended in lysis buffer. Proteins were measured by using Pierce BCA Protein Assay kit (Thermo scientific, Rockford, Ill., USA). Approximately 10 μg (MP) and 30-40 μg (HepG2) of proteins were resolved by a 4-20% Criterion Tris-HCl gel electrophoresis (Biorad, Hercules, Calif., USA) and transferred to a polyvinylidene difluoride (PVDF) membrane (Biorad, Hercules, Calif., USA). Membranes were blocked for 1 hour at room temperature with 3-5% low-fat milk (Biorad, Hercules, Calif., USA) in 1×PBS, 0.1% Tween 20 (PBS-T). Primary rabbit polyclonal antibody anti-human VNN1 (ID: 8876; Epitomics), anti-mouse VNN1 (Proteintech, San Diego, Calif., USA), anti-VCAM-1 (ID: 7412; Santa Cruz Biotechnology, Santa Cruz, Calif., USA), mouse monoclonal antibody anti-ICAM-1 (ID: 3383; Abnova, Taipei, Taiwan) and anti-GAPDH (ID: 2597; Cell Signaling, Boston, Mass., USA) were incubated overnight at 4° C. After washing with PBS-T, membranes were incubated with goat anti-rabbit or mouse secondary antibody and proteins were visualized by Supersignal West Pico chemiluminescence substrate (Pierce biotechnology, Rockford, Ill., USA). A densitometry analysis of the specific protein bands was performed by using Image J (Version 1.4.3.67).

### Statistical Analysis

All data were expressed as the mean±SD unless otherwise indicated. Differences between three or more groups were compared by an ANOVA analysis followed by a post-hoc Newman-Keuls test, parametric test or the Kruskall-Wallis nonparametric test. Differences between two groups of normalized data were compared by a Student's t-test. Differences were considered to be statistically significant at P<0.05. All statistical analysis was performed using GraphPad Prism 4.0c (La Jolla, Calif., USA).

### REFERENCES

1. A. Alisi, A. E. Feldstein, A. Villani, M. Raponi, V. Nobili, Pediatric nonalcoholic fatty liver disease: a multidisciplinary approach. Nature reviews. Gastroenterology & hepatology 9, 152-161 (2012).
2. P. Angulo, Nonalcoholic fatty liver disease. The New England journal of medicine 346, 1221-1231 (2002).
3. A. Wieckowska, A. E. Feldstein, Nonalcoholic fatty liver disease in the pediatric population: a review. Current opinion in pediatrics 17, 636-641 (2005).
4. E. M. Brunt, Pathology of fatty liver disease. Modern pathology: an official journal of the United States and Canadian Academy of Pathology, Inc 20 Suppl 1, S40-48 (2007).
5. L. A. Adams, J. F. Lymp, J. St Sauver, S. O. Sanderson, K. D. Lindor, A. Feldstein, P. Angulo, The natural history of nonalcoholic fatty liver disease: a population-based cohort study. Gastroenterology 129, 113-121 (2005).
6. M. S. Ascha, I. A. Hanouneh, R. Lopez, T. A. Tamimi, A. F. Feldstein, N. N. Zein, The incidence and risk factors of hepatocellular carcinoma in patients with nonalcoholic steatohepatitis. Hepatology 51, 1972-1978 (2010).
7. S. Coulon, F. Heindryckx, A. Geerts, C. Van Steenkiste, I. Colle, H. Van Vlierberghe, Angiogenesis in chronic liver disease and its complications. Liver international: official journal of the International Association for the Study of the Liver 31, 146-162 (2011).
8. Y. Yilmaz, O. Yonal, R. Kurt, Y. O. Alandab, O. Ozdogan, C. A. Celikel, E. Ulukaya, N. Imeryuz, C. Kalayci, E. Avsar, Circulating levels of vascular endothelial growth factor A and its soluble receptor in patients with biopsy-proven nonalcoholic fatty liver disease. Archives of medical research 42, 38-43 (2011).
9. M. Kitade, H. Yoshiji, H. Kojima, Y. Ikenaka, R. Noguchi, K. Kaji, J. Yoshii, K. Yanase, T. Namisaki, K. Asada, M. Yamazaki, T. Tsujimoto, T. Akahane, M. Uemura, H. Fukui, Leptin-mediated neovascularization is a prerequisite for progression of nonalcoholic steatohepatitis in rats. Hepatology 44, 983-991 (2006).
10. M. Kitade, H. Yoshiji, H. Kojima, Y. Ikenaka, R. Noguchi, K. Kaji, J. Yoshii, K. Yanase, T. Namisaki, M. Yamazaki, T. Tsujimoto, K. Moriya, H. Kawaratani, T. Akahane, H. Fukui, Neovascularization and oxidative stress in the progression of non-alcoholic steatohepatitis. Molecular medicine reports 1, 543-548 (2008).
11. H. Yoshiji, R. Noguchi, Y. Ikenaka, T. Namisaki, M. Kitade, K. Kaji, Y. Shirai, J. Yoshii, K. Yanase, M. Yamazaki, T. Tsujimoto, H. Kawaratani, T. Akahane, Y. Aihara, H. Fukui, Losartan, an angiotensin-II type 1 receptor blocker, attenuates the liver fibrosis development of non-alcoholic steatohepatitis in the rat. BMC research notes 2, 70 (2009).
12. M. Kitade, H. Yoshiji, R. Noguchi, Y. Ikenaka, K. Kaji, Y. Shirai, M. Yamazaki, M. Uemura, J. Yamao, M. Fujimoto, A. Mitoro, M. Toyohara, M. Sawai, M. Yoshida, C. Morioka, T. Tsujimoto, H. Kawaratani, H. Fukui, Crosstalk between angiogenesis, cytokeratin-18, and insulin resistance in the progression of non-alcoholic steatohepatitis. World journal of gastroenterology: WJG 15, 5193-5199 (2009).
13. J. D. Browning, J. D. Horton, Molecular mediators of hepatic steatosis and liver injury. The Journal of clinical investigation 114, 147-152 (2004).
14. M. Trauner, M. Arrese, M. Wagner, Fatty liver and lipotoxicity. Biochimica et biophysicaacta 1801, 299-310 (2010).
15. A. E. Feldstein, N. W. Werneburg, A. Canbay, M. E. Guicciardi, S. F. Bronk, R. Rydzewski, L. J. Burgart, G. J. Gores, Free fatty acids promote hepatic lipotoxicity by stimulating TNFalpha expression via a lysosomal pathway. Hepatology 40, 185-194 (2004).
16. Z. Z. Li, M. Berk, T. M. McIntyre, A. E. Feldstein, Hepatic lipid partitioning and liver damage in nonalcoholic fatty liver disease: role of stearoyl-CoA desaturase. The Journal of biological chemistry 284, 5637-5644 (2009).
17. B. Gyorgy, T. G. Szabo, M. Pasztoi, Z. Pal, P. Misjak, B. Aradi, V. Laszlo, E. Pallinger, E. Pap, A. Kittel, G. Nagy, A. Falus, E. I. Buzas, Membrane vesicles, current state-of-the-art: emerging role of extracellular vesicles. Cellular and molecular life sciences: CMLS 68, 2667-2688 (2011).
18. E. L. A. S, I. Mager, X. O. Breakefield, M. J. Wood, Extracellular vesicles: biology and emerging therapeutic opportunities. Nature reviews. Drug discovery 12, 347-357 (2013).
19. M. L. Coleman, E. A. Sahai, M. Yeo, M. Bosch, A. Dewar, M. F. Olson, Membrane blebbing during apoptosis results from caspase-mediated activation of ROCK I. Nature cell biology 3, 339-345 (2001).
20. M. Sebbagh, C. Renvoize, J. Hamelin, N. Riche, J. Bertoglio, J. Breard, Caspase 3-mediated cleavage of ROCK I induces MLC phosphorylation and apoptotic membrane blebbing. Nature cell biology 3, 346-352 (2001).
21. G. Pitari, F. Malergue, F. Martin, J. M. Philippe, M. T. Massucci, C. Chabret, B. Maras, S. Dupre, P. Naquet, F. Galland, Pantetheinase activity of membrane-bound Vanin-1: lack of free cysteamine in tissues of Vanin-1 deficient mice. FEBS letters 483, 149-154 (2000).
22. C. Berruyer, L. Pouyet, V. Millet, F. M. Martin, A. LeGoffic, A. Canonici, S. Garcia, C. Bagnis, P. Naquet, F. Galland, Vanin-1 licenses inflammatory mediator production by gut epithelial cells and controls colitis by antagonizing peroxisome proliferator-activated receptor gamma activity. The Journal of experimental medicine 203, 2817-2827 (2006).
23. K. Simons, D. Toomre, Lipid rafts and signal transduction. Nature reviews. Molecular cell biology 1, 31-39 (2000).
24. N. Chazal, D. Gerlier, Virus entry, assembly, budding, and membrane rafts. Microbiology and molecular biology reviews: MMBR 67, 226-237, table of contents (2003).
25. H. H. Patel, P. A. Insel, Lipid rafts and caveolae and their role in compartmentation of redox signaling. Antioxidants & redox signaling 11, 1357-1372 (2009).
26. K. J. Svensson, H. C. Christianson, A. Wittrup, E. Bourseau-Guilmain, E. Lindqvist, L. M. Svensson, M. Morgelin, M. Belting, Exosome Uptake Depends on ERK1/2-Heat Shock Protein Signaling and Lipid Raft-mediated Endocytosis Negatively Regulated by Caveolin-1. The Journal of biological chemistry 288, 17713-17724 (2013).
27. H. Park, Y. M. Go, R. Darji, J. W. Choi, M. P. Lisanti, M. C. Maland, H. Jo, Caveolin-1 regulates shear stress-dependent activation of extracellular signal-regulated kinase. American journal of physiology. Heart and circulatory physiology 278, H1285-1293 (2000).
28. A. Koteish, A. M. Diehl, Animal models of steatosis. Seminars in liver disease 21, 89-104 (2001).
29. A. E. Christian, M. P. Haynes, M. C. Phillips, G. H. Rothblat, Use of cyclodextrins for manipulating cellular cholesterol content. Journal of lipid research 38, 2264-2272 (1997).
30. S. Sabharanjak, P. Sharma, R. G. Parton, S. Mayor, GPI-anchored proteins are delivered to recycling endosomes via a distinct cdc42-regulated, clathrin-independent pinocytic pathway. Developmental cell 2, 411-423 (2002).
31. S. E. Lakhan, S. Sabharanjak, A. De, Endocytosis of glycosylphosphatidylinositol-anchored proteins. Journal of biomedical science 16, 93 (2009).
32. H. Nakabayashi, K. Shimizu, HA1077, a Rho kinase inhibitor, suppresses glioma induced angiogenesis by targeting the Rho-ROCK and the mitogen-activated protein kinase kinase/extracellular signal-regulated kinase (MEK/ERK) signal pathways. Cancer science 102, 393-399 (2011).
33. K. J. Dammanahalli, S. Stevens, R. Terkeltaub, Vanin-1 pantetheinase drives smooth muscle cell activation in post-arterial injury neointimal hyperplasia. PloS one 7, e39106 (2012).
34. J. Liebl, S. B. Weitensteiner, G. Vereb, L. Takacs, R. Furst, A. M. Vollmar, S. Zahler, Cyclin-dependent kinase 5 regulates endothelial cell migration and angiogenesis. The Journal of biological chemistry 285, 35932-35943 (2010).
35. A. Rogue, C. Lambert, R. Josse, S. Antherieu, C. Spire, N. Claude, A. Guillouzo, Comparative gene expression profiles induced by PPARgamma and PPARalpha/gamma agonists in human hepatocytes. PloS one 6, e18816 (2011).
36. J. H. Fu, H. S. Sun, Y. Wang, W. Q. Zheng, Z. Y. Shi, Q. J. Wang, The effects of a fat- and sugar-enriched diet and chronic stress on nonalcoholic fatty liver disease in male Wistar rats. Digestive diseases and sciences 55, 2227-2236 (2010).
37. F. A. Nascimento, S. Barbosa-da-Silva, C. Fernandes-Santos, C. A. Mandarim-de-Lacerda, M. B. Aguila, Adipose tissue, liver and pancreas structural alterations in C57BL/6 mice fed high-fat-high-sucrose diet supplemented with fish oil (n-3 fatty acid rich oil). Experimental and toxicologic pathology: official journal of the Gesellschaft fur Toxikologische Pathologie 62, 17-25 (2010).
38. M. Castoldi, M. Vujic Spasic, S. Altamura, J. Elmen, M. Lindow, J. Kiss, J. Stoke, R. Sparla, L. A. D'Alessandro, U. Klingmuller, R. E. Fleming, T. Longerich, H. J. Grone, V. Benes, S. Kauppinen, M. W. Hentze, M. U. Muckenthaler, The liver-specific microRNA miR-122 controls systemic iron homeostasis in mice. The Journal of clinical investigation 121, 1386-1396 (2011).
39. S. Bala, J. Petrasek, S. Mundkur, D. Catalano, I. Levin, J. Ward, H. Alao, K. Kodys, G. Szabo, Circulating microRNAs in exosomes indicate hepatocyte injury and inflammation in alcoholic, drug-induced, and inflammatory liver diseases. Hepatology 56, 1946-1957 (2012).
40. C. Paternostro, E. David, E. Novo, M. Parola, Hypoxia, angiogenesis and liver fibrogenesis in the progression of chronic liver diseases. World journal of gastroenterology: WJG 16, 281-288 (2010).
41. S. Coulon, V. Legry, F. Heindryckx, C. Van Steenkiste, C. Casteleyn, K. Olievier, L. Libbrecht, P. Carmeliet, B. Jonckx, J. M. Stassen, H. Van Vlierberghe, I. Leclercq, I. Colle, A. Geerts, Role of vascular endothelial growth factor in the pathophysiology of nonalcoholic steatohepatitis in two rodent models. Hepatology 57, 1793-1805 (2013).
42. N. I. Moldovan, Role of monocytes and macrophages in adult angiogenesis: a light at the tunnel's end. Journal of hematotherapy & stem cell research 11, 179-194 (2002).
43. E. Giraudo, M. Inoue, D. Hanahan, An amino-bisphosphonate targets MMP-9-expressing macrophages and angiogenesis to impair cervical carcinogenesis. The Journal of clinical investigation 114, 623-633 (2004).
44. O. Morel, N. Morel, L. Jesel, J. M. Freyssinet, F. Toti, Microparticles: a critical component in the nexus between inflammation, immunity, and thrombosis. Seminars in immunopathology 33, 469-486 (2011).
45. P. E. Rautou, A. C. Vion, N. Amabile, G. Chironi, A. Simon, A. Tedgui, C. M. Boulanger, Microparticles, vascular function, and atherothrombosis. Circulation research 109, 593-606 (2011).
46. P. E. Rautou, J. Bresson, Y. Sainte-Marie, A. C. Vion, V. Paradis, J. M. Renard, C. Devue, C. Heymes, P. Letteron, L. Elkrief, D. Lebrec, D. Valla, A. Tedgui, R. Moreau, C. M. Boulanger, Abnormal plasma microparticles impair vasoconstrictor responses in patients with cirrhosis. Gastroenterology 143, 166-176 e166 (2012).
47. R. P. Witek, L. Yang, R. Liu, Y. Jung, A. Omenetti, W. K. Syn, S. S. Choi, Y. Cheong, C. M. Fearing, K. M. Agboola, W. Chen, A. M. Diehl, Liver cell-derived microparticles activate hedgehog signaling and alter gene expression in hepatic endothelial cells. Gastroenterology 136, 320-330 e322 (2009).
48. M. Kornek, M. Lynch, S. H. Mehta, M. Lai, M. Exley, N. H. Afdhal, D. Schuppan, Circulating microparticles as disease-specific biomarkers of severity of inflammation in patients with hepatitis C or nonalcoholic steatohepatitis. Gastroenterology 143, 448-458 (2012).
49. L. Miguet, K. Pacaud, C. Felden, B. Hugel, M. C. Martinez, J. M. Freyssinet, R. Herbrecht, N. Potier, A. van Dorsselaer, L. Mauvieux, Proteomic analysis of malignant lymphocyte membrane microparticles using double ionization coverage optimization. Proteomics 6, 153-171 (2006).
50. D. B. Peterson, T. Sander, S. Kaul, B. T. Wakim, B. Halligan, S. Twigger, K. A. Pritchard, Jr., K. T. Oldham, J. S. Ou, Comparative proteomic analysis of PAI-1 and TNF-alpha derived endothelial microparticles. Proteomics 8, 2430-2446 (2008).
51. M. Prokopi, G. Pula, U. Mayr, C. Devue, J. Gallagher, Q. Xiao, C. M. Boulanger, N. Westwood, C. Urbich, J. Willeit, M. Steiner, J. Breuss, Q. Xu, S. Kiechl, M. Mayr, Proteomic analysis reveals presence of platelet microparticles in endothelial progenitor cell cultures. Blood 114, 723-732 (2009).
52. J. Conde-Vancells, E. Rodriguez-Suarez, N. Embade, D. Gil, R. Matthiesen, M. Valle, F. Elortza, S. C. Lu, J. M. Mato, J. M. Falcon-Perez, Characterization and comprehensive proteome profiling of exosomes secreted by hepatocytes. Journal of proteome research 7, 5157-5166 (2008).
53. A. A. Nanji, Animal models of nonalcoholic fatty liver disease and steatohepatitis. Clinics in liver disease 8, 559-574, ix (2004).
54. M. Woo, R. Hakem, M. S. Soengas, G. S. Duncan, A. Shahinian, D. Kagi, A. Hakem, M. McCurrach, W. Khoo, S. A. Kaufman, G. Senaldi, T. Howard, S. W. Lowe, T. W. Mak, Essential contribution of caspase 3/CPP32 to apoptosis and its associated nuclear changes. Genes & development 12, 806-819 (1998).
55. R. Kohli, A. E. Feldstein, NASH animal models: are we there yet? Journal of hepatology 55, 941-943 (2011).
56. M. Kornek, Y. Popov, T. A. Libermann, N. H. Afdhal, D. Schuppan, Human T cell microparticles circulate in blood of hepatitis patients and induce fibrolytic activation of hepatic stellate cells. Hepatology 53, 230-242 (2011).
57. C. G. Havens, A. Ho, N. Yoshioka, S. F. Dowdy, Regulation of late G1/S phase transition and APC Cdh1 by reactive oxygen species. Molecular and cellular biology 26, 4701-4711 (2006).
58. S. Wada, S. Obika, M. A. Shibata, T. Yamamoto, M. Nakatani, T. Yamaoka, H. Torigoe, M. Harada-Shiba, Development of a 2′,4′-BNA/LNA-based siRNA for Dyslipidemia and Assessment of the Effects of Its Chemical Modifications In vivo. Molecular therapy. Nucleic acids 1, e45 (2012).
59. M. L. Torgersen, G. Skretting, B. van Deurs, K. Sandvig, Internalization of cholera toxin by different endocytic mechanisms. Journal of cell science 114, 3737-3747 (2001).
60. M. Guttman, G. N. Betts, H. Barnes, M. Ghassemian, P. van der Geer, E. A. Komives, Interactions of the NPXY microdomains of the low density lipoprotein receptor-related protein 1. Proteomics 9, 5016-5028 (2009).

61. A. L. McCormack, D. M. Schieltz, B. Goode, S. Yang, G. Barnes, D. Drubin, J. R. Yates, 3rd, Direct analysis and identification of proteins in mixtures by LC/MS/MS and database searching at the low-femtomole level. Analytical chemistry 69: 767-776 (1997).

### Example 2

### Characterization of Circulating Extracellular Vesicles in Nash Development

Metabolic non-alcohol related Fatty Liver Disease (NAFLD) has become the most common form of chronic liver disease in both children and adults affecting up to 30% of the American Population [1, 2]. Mexican-Americans are at particular risk for this disease, which is now the fastest growing indicator for liver transplantation eligibility [3, 4]. NAFLD is tightly associated with obesity and encompasses a wide spectrum of conditions associated with over accumulation of fat in the liver, ranging from hepatic steatosis to steatohepatitis (NASH) and cirrhosis [5]. Hepatic steatosis is characterized by isolated accumulation of lipids in the liver and is generally thought to follow a relatively benign non-progressive clinical course [6, 7].

NASH is a serious condition, with about 5 to 25% of patients progressing to fibrosis and cirrhosis with its associated complications of portal hypertension, liver failure and hepatocellular carcinoma [5, 8]. Liver biopsy, an invasive procedure associated with possible significant complications, remains at present time the only reliable method to differentiate hepatic steatosis from NASH [9, 10]. It is also the only way to monitor any response to therapeutic interventions. Therefore, there is currently an urgent need to develop noninvasive tests for this condition.

Extracellular vesicles (EVs) are small membrane vehicles released in a highly regulated manner from dying or activated cells. There are two main populations of EVs, namely exosomes and microparticles (MPs), which differ in their size, composition and mechanisms of generation. Exosomes are small, 30-100 nm in diameter, and are released by exocytosis as a result of fusion of multivesicular bodies with the plasma membrane. MPs are between 100-1000 nm in size and are generated through cell membrane shedding in a process that involves a regulated sorting of membrane proteins into the shed MP and the flipping of phosphatidylserine from the inner to the outer membrane during cellular activation or early apoptosis [11, 12]. EVs are key cell-to-cell communicators because EVs have signatures from parenteral cells such as surface receptors, integral membrane proteins, cytosolic and nuclear proteins, RNAs (including miRNAs) [13-15] and deliver these signatures to other cells through interaction with surface receptors or internalization [16, 17]. Notably, released EVs do not only stay in the tissue of origin, but also circulate in the blood stream. Indeed, recent studies have demonstrated that primary murine hepatocytes, as well as different hepatocyte cell lines, are capable of producing and releasing the two main subtypes of EVs: exosomes and MPs [18-21]. In vivo studies in bile duct-ligated rats have found increased circulating MPs, while two recent pilot studies in humans showed increased levels of inflammatory cell derived MPs in patients with NAFLD and in patients with alcohol and/or chronic hepatitis C related cirrhosis [18, 22-24].

Thus, the studies provided herewith were: 1) to examine whether EVs are increased in liver and blood during experimental NASH; 2) to perform detailed characterization of the type of EV population released during NASH development by using electron microscopy (EM), dynamic light scattering analysis, flow cytometry and LC MS/MS proteomic analysis; and 3) assess the utility of monitoring and quantifying EVs in blood as biomarkers of liver damage in NASH. The results demonstrated a marked increase in circulating EVs in NASH and established the antigenic composition of circulating EVs. Moreover, characterization of EVs identified both exosomes and microparticles released in experimental NASH, with the latter being the most predominant. Finally, time course experiments showed that blood EV levels are dynamic and strongly correlate with liver histopathological features. These results suggest that circulating EVs could novel noninvasive biomarkers to monitor liver damage in NASH.

The detailed results of the studies are provided as follows:

Diet-Induced NASH Results in the Production of Extracellular Vesicles that are Composed Mainly of Microparticles

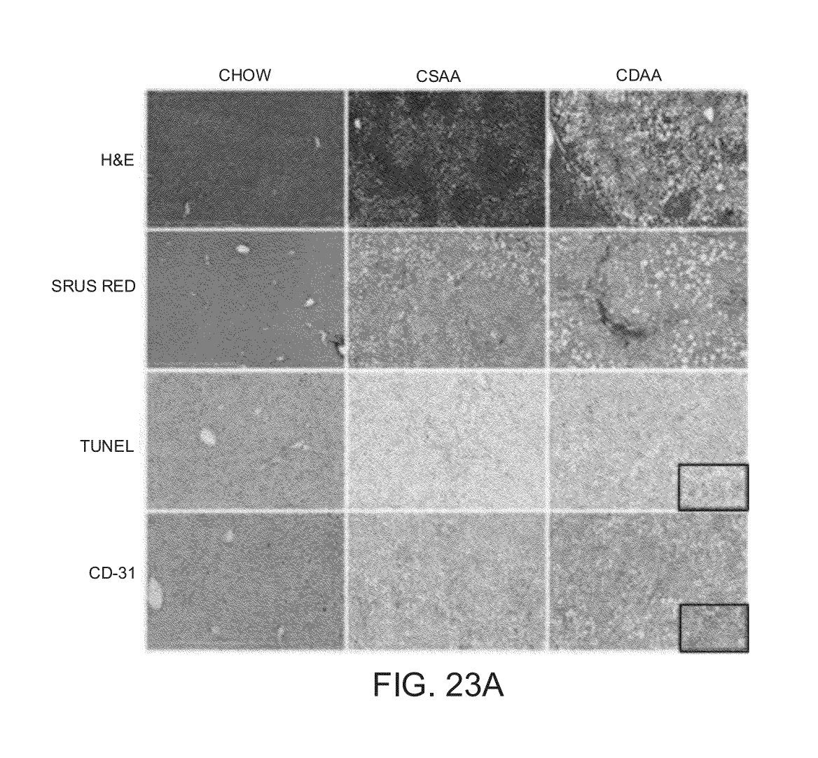

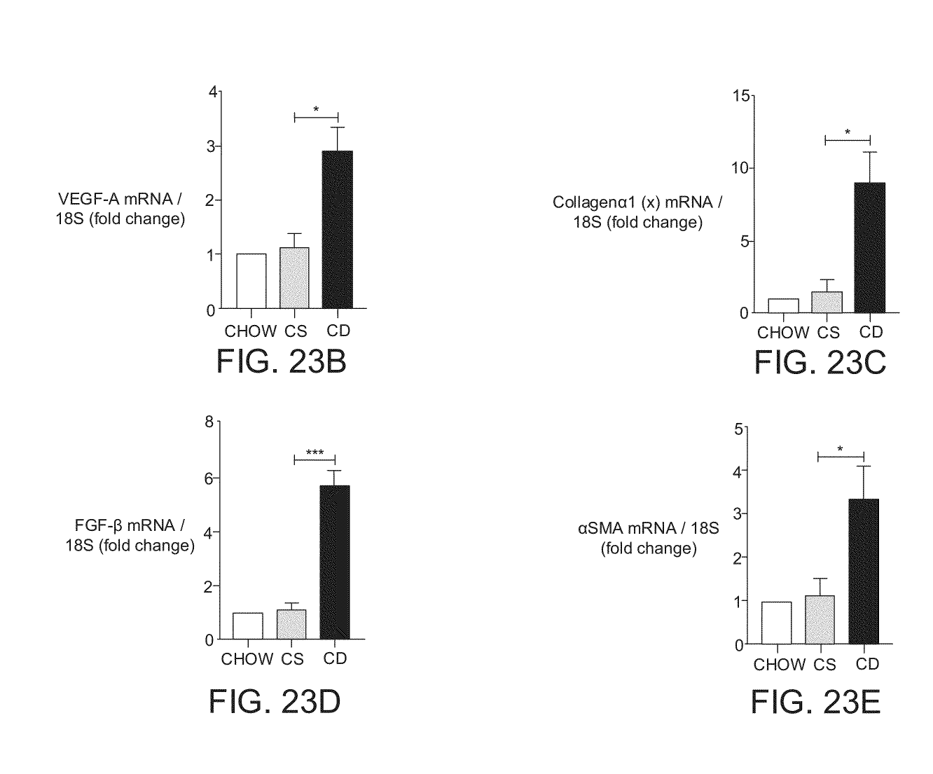

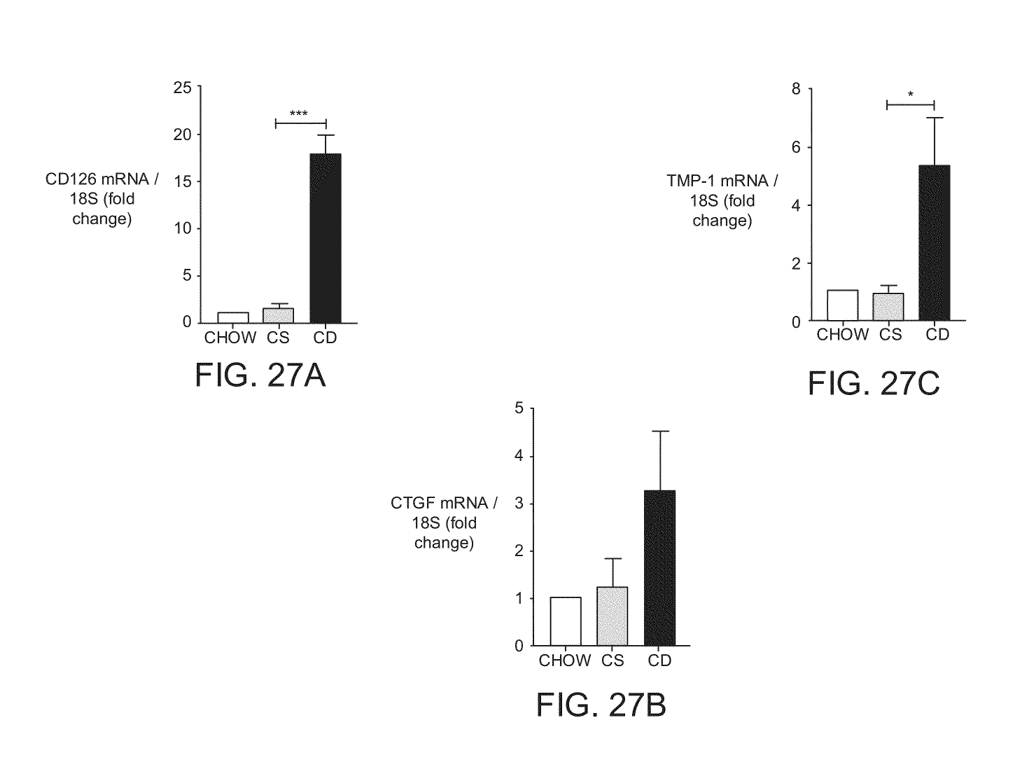

Lipotoxicity and cell death play critical roles in NASH development [6]. To investigate whether EVs are produced and released during NASH, male C57BL/6 mice were placed on a Choline Deficient L-Amino Acid (CDAA) diet-induced NASH, a Choline Supplemented LAmino Acid (CSAA) diet or a regular Chow diet for 20 weeks. Mice on the CDAA diet developed significant predominantly macro-vesicular steatosis, marked inflammation, cell death, pathological angiogenesis and fibrosis, as shown by the histopathological and quantitative-PCR analyses (FIGS. 23 and 27).

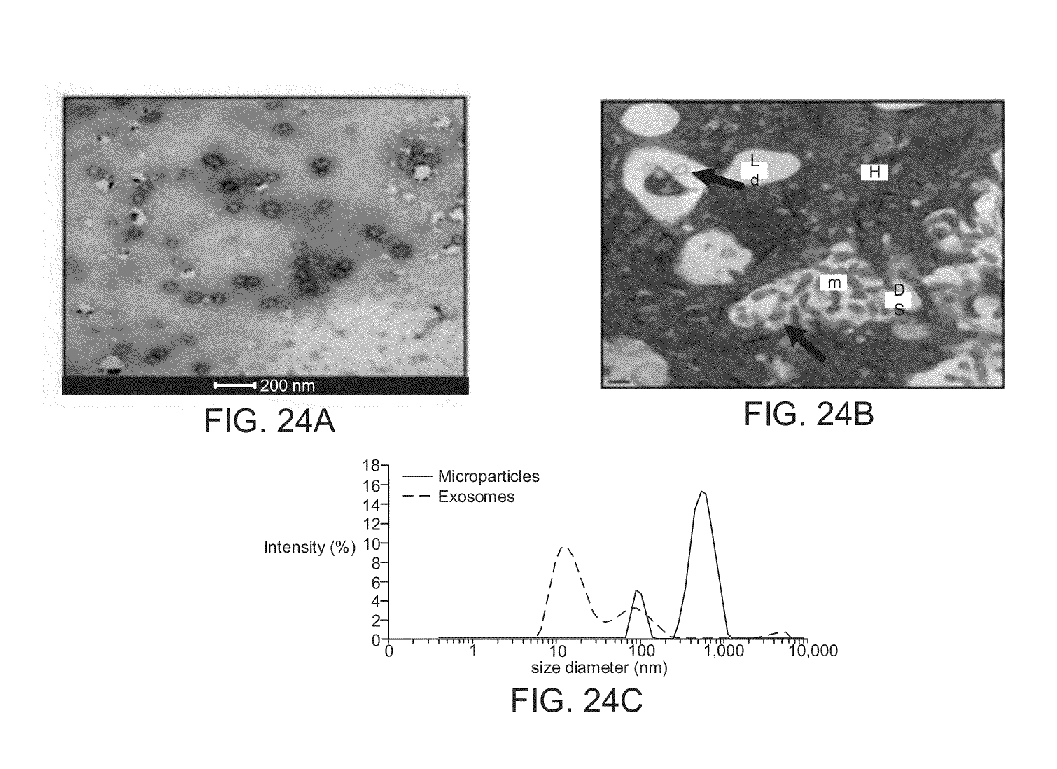

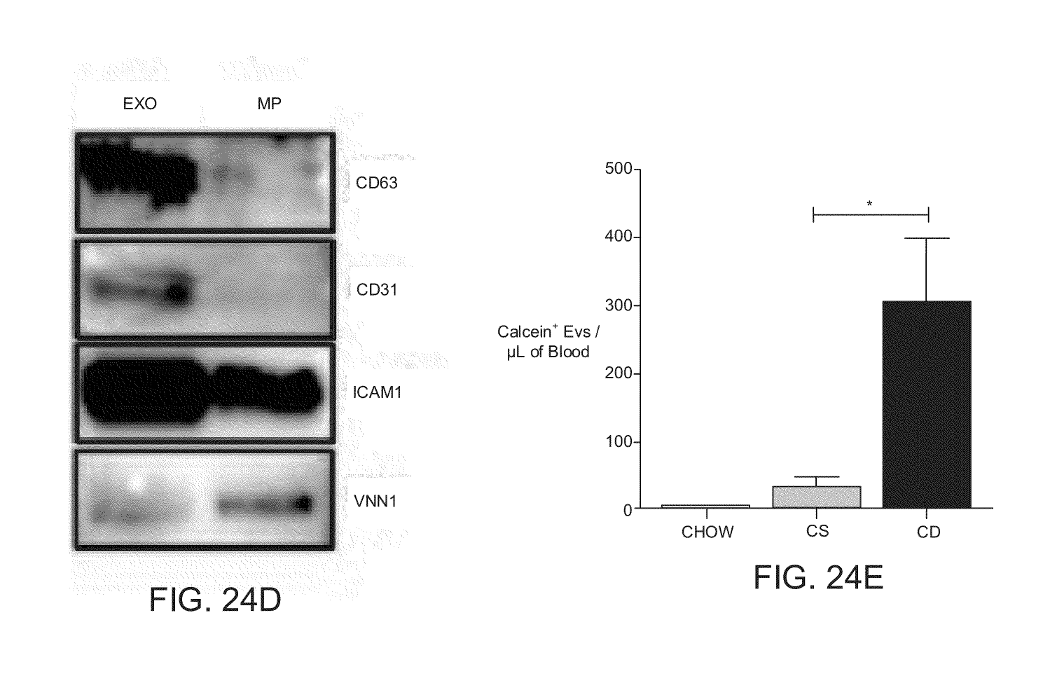

Electron microscopy demonstrated the presence of EVs in the livers and blood of mice with NASH (FIGS. 24A-B). Importantly, it was observed that EVs were predominantly detected in the space of Disse, between the hepatocyte villi and the sinusoid wall (FIG. 24B). Next, series of studies including dynamic light scattering analysis and FACS analysis were performed, which identified both microparticles and exosomes in the EVs population released in circulation of CDAA treated mice (FIG. 24C). Indeed, analysis of EV size identified two distinct peaks, a large predominant peak that corresponded to EVs with a diameter between 100 and 1,000 nm (mean 530 nm) consistent with the size previously reported for MPs [24], and a second small peak of EVs with a diameter between 30 and 100 nm (mean 50 nm) consistent with the size previously reported for exosomes [25, 26] (FIG. 24C). In order to confirm the presence of exosome (EXO) and microparticle (MP) populations within the total purified circulating extracellular vesicles isolated from the CDAA-fed mice for 20 weeks, a western blotting analysis was performed for several known exosomal markers (Cd63, Cd81, Icam1) or for Vanin-1 (VNN1), a novel surface ectoenzyme which has been previously detected in circulating microparticles in a methionine and choline deficient (MCD) diet-induced experimental NASH [19] (FIG. 24D). Additionally, the FACS analysis identified a marked increase in calcein-positive EVs in blood from CDAA treated mice compared to CSAA and chow treated mice (2 vs. 34.3 vs. 456.5 EVs/μL plasma; p<0.03) (FIG. 24E).

### Antigenic Composition of Blood Extracellular Vesicles Released During NASH

Due to the findings that EVs are released in blood and livers during NASH, the characterization of the antigenic composition of blood EVs in NASH was focused on. Indeed, in order to identify potential protein targets present in blood EVs during NASH development, a comprehensive proteomic analysis of purified blood EVs was performed. For these studies purified EVs were obtained from platelet-free plasma collected from mice fed the CDAA or control diets for 20 weeks, by using a sucrose gradient ultracentrifugation. The protein content of isolated blood EVs was then assessed by using LC-MS/MS analysis. Importantly, the proteome of blood EVs isolated from CDAA mice differed from that of control diet mice, where only several keratins, albumin or actin isoforms were detected (Table 4).

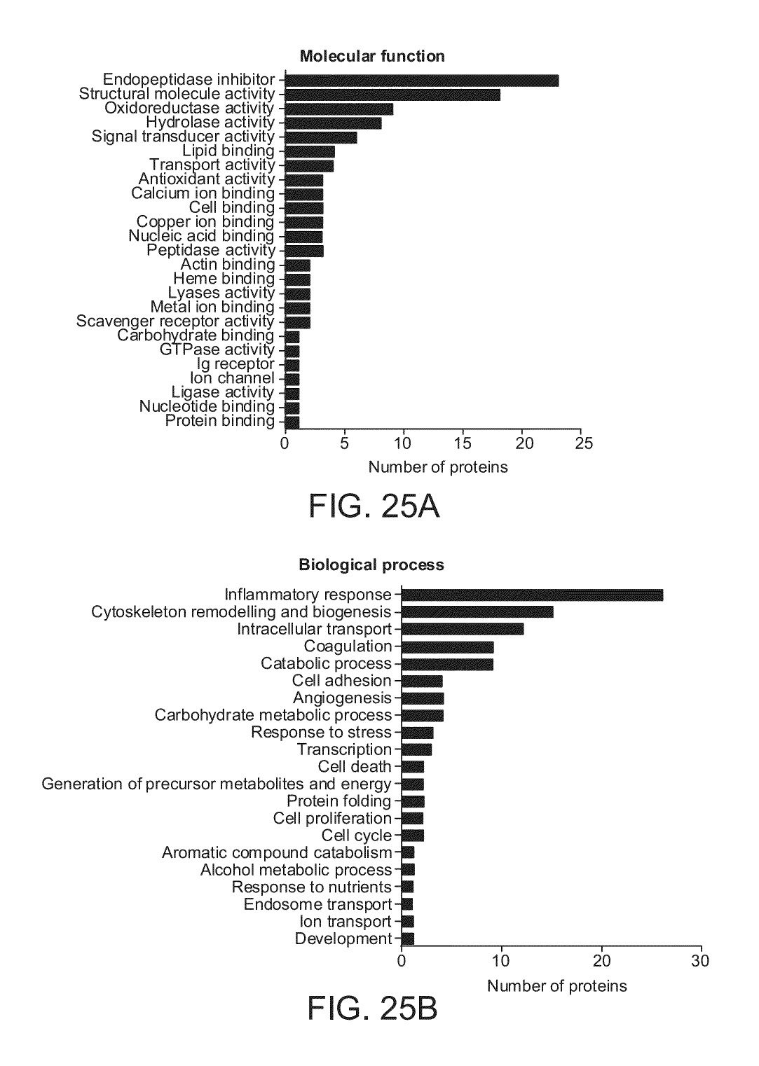

Table 3 lists all of the 106 unique proteins identified in three independent preparations of blood EVs isolated from CDAA fed mice. The 106 proteins identified from circulating EVs from CDAA fed mice for 20 weeks, could be assigned to Genome Ontology (GO) categories which are defined by the GO Consortium [27]. Based on the GO analysis of the newly discovered proteins, blood EVs carry cytosolic, extracellular, plasma membrane, and nuclear proteins. It was found that some functional activity of proteins such as oxidoreductase, hydrolase, endopeptidase inhibitors, signal transducers and lipid binding were abundantly over-expressed in the pure EVs preparation, indicating that these activities could be of important relevance regarding the potential effect of the circulating EVs on the target cells or tissue (FIG. 25). Moreover, the analysis identified a significant amount of proteins involved particularly in inflammation and immunity, cell death, angiogenesis and cell adhesion, which represent all important features of NASH.

These results suggest that the circulating EVs carry a variety of different proteins, different in molecular function and biological process, and that reflect the pathological progression of NASH.

TABLE 3
Proteins identified in the CDAA-fed mice circulating extracellular vesicles by LC-MS/MS

| | Gene | | | Peptides | | |
|---|---|---|---|---|---|---|
| | symbol | Gene ID | Protein Description | (95%) ^{a} | % Cov ^{b} | SpC ^{c} |
| Cytoskeletal | KRT6A | 16687 | Keratin, 6A, type II | 20 | 47.87 | 47 |
| remodelling/ | ACTG1 | 11465 | Actin, gamma, 1 | 5 | 23.73 | 16 |
| vesiculation | ACTA2 | 11475 | Actin, aortic smooth muscle | 1 | 7.16 | 1 |
| | PLAK | 16480 | Junction plakoglobin | 4 | 8.32 | 6 |
| | KRT5 | 110308 | Keratin 5 | 22 | 34.48 | 46 |
| | GSN | 227753 | Gelsolin | 3 | 28.08 | 7 |
| | KRT77 | 406220 | Keratin 77 | 4 | 18.18 | 24 |
| | KRT17 | 16667 | Keratin 17, type I | 10 | 39.26 | 26 |
| | KRT25 | 70810 | Keratin 25, type I | 1 | 12.44 | 11 |
| | KRT1 | 16678 | Keratin 1, type II | 5 | 13.97 | 63 |
| | KRT2 | 16681 | Keratin 2, type II | 9 | 22.49 | 24 |
| | KRT73 | 223915 | Keratin 73, type II | 6 | 22.08 | 21 |
| | KRT10 | 16661 | Keratin 10 | 7 | 12.11 | 100 |
| | NEST | 18008 | Nestin | 1 | 2 | 2 |
| | KRT6B | 16688 | Keratin 6B, type II | 5 | 6.01 | 26 |
| | KRT42 | 68239 | Keratin 42, type I | 4 | 8 | 8 |
| Signalling/ | PRDX2 | 21672 | Peroxiredoxin-2 | 2 | 9.59 | 3 |
| Chaperone/ | REFBP2 | 56009 | RNA and export factor-binding protein 2 | 1 | 15.14 | 2 |
| Transcription | TSKU | 244152 | Tsukushin | 1 | 9.6 | 1 |
| regulation | APCS | 20219 | Serum amyloid P-component | 1 | 24.11 | 3 |
| | BACH1 | 12013 | Transcription regulator protein BACH1 | 1 | 5.82 | 1 |
| | HMGA1 | 15361 | High mobility group AT-hook 1 | 1 | 23.36 | 1 |
| | UBC | 22190 | Ubc protein | 1 | 19.24 | 3 |
| | CHRNA10 | 504186 | Cholinergic receptor, nicotinic, alpha polypeptide 10 | 1 | 2.01 | 1 |
| | ASAP2 | 211914 | Arf-GAP with SH3, PH domains, ANK repeat 2 | 1 | 0.82 | 3 |
| | PRDX1 | 18477 | Peroxiredoxin-1 | 2 | 14.06 | 4 |
| | PRDX4 | 53381 | Peroxiredoxin-4 | 1 | 3.49 | 2 |
| Enxymes/ | FABP5 | 16592 | Fatty acid binding protein 5 | 1 | 6.66 | 1 |
| Metabolic | ALDH1A7 | 26358 | Aldehyde dehydrogenase, cytosolic 1 | 3 | 22.75 | 12 |
| processes | ALDOA | 11674 | Fructose-bisphosphate aldolase | 1 | 6.31 | 1 |
| | RBP-4 | 19662 | Retinol-binding protein 4 | 1 | 4.97 | 2 |
| | ALDH1A1 | 11668 | Retinal dehydrogenase I | 3 | 24.35 | 12 |
| | SERPIN-A1 | 20700 | Alpha-1-antitrypsin 1-1 (Serpin A1) | 24 | 50.12 | 122 |
| | TTR | 22139 | Transthyretin | 11 | 79.59 | 62 |
| | PSMB1 | 19170 | Proteasome subunit beta type | 7 | 39.17 | 15 |
| | PSMA5 | 26442 | Proteasome subunit alpha type | 3 | 34.44 | 7 |
| | HBB-A1 | 15122 | Alpha-globin 1 | 20 | 85.21 | 95 |
| | HBB-B1 | 15129 | Beta-globin 1 | 12 | 57.14 | 88 |
| | GC | 14473 | Vitamin D-binding protein | 4 | 22.69 | 11 |
| | PON1 | 18979 | Serum paraoxonase/arylesterase 1 | 3 | 21.97 | 4 |
| | H6PD | 100198 | GDH/6PGL endoplasmic bifunctional protein | 1 | 5.07 | 1 |
| | GPLD1 | 14756 | Glycosylphosphatidylmositol specific phospholipase D1 | 4 | 11.4 | 11 |
| | MGAM | 232714 | Maltase-glucoamylase | 16 | 14.89 | 47 |
| | GAPDH | 14433 | Glyceraldehyde-3-phosphate dehydrogenase | 1 | 12.46 | 1 |
| | GPX3 | 14778 | Glutathione peroxidase 3 | 5 | 34.96 | 18 |
| | ME1 | 17436 | Malic enzyme | 1 | 13.81 | 2 |
| | CAR2 | 12349 | Carbonic anhydrase 2 | 1 | 6.15 | 4 |
| | SERPIN-N3 | 20716 | Serine protease inhibitor A3N | 3 | 22.01 | 6 |
| | SERPIN-K3 | 20714 | Serine protease inhibitor A3K | 16 | 41.39 | 81 |
| | SERPIN-A6 | 12401 | Corticosteroid-binding globulin | 1 | 4.78 | 3 |
| | CP | 12870 | Ceruloplasmin | 31 | 34.31 | 139 |
| | CPN1 | 93721 | Carboxypeptidase N catalytic chain | 3 | 14.88 | 9 |
| | LAP3 | 66988 | Cytosol aminopeptidase | 2 | 15.99 | 3 |
| | CANT1 | 76025 | Calcium activated nucleotidase 1 | 1 | 3.18 | 2 |
| | ALB | 11657 | Albumin | 114 | 70.07 | 7986 |
| | APOA4 | 11808 | Apolipoprotein A-IV | 6 | 23.29 | 8 |
| | DNPEP | 13437 | Aspartyl aminopeptidase | 1 | 25.53 | 3 |
| | APOA2 | 11807 | Apolipoprotein A-II | 15 | 76.47 | 36 |
| | HP | 15439 | Haptoglobin | 13 | 51.87 | 115 |
| | OXR1 | 170719 | Oxidation resistance protein 1 | 1 | 1.27 | 2 |
| Fibrinolysis/ | A2M | 232345 | Alpha-2-Macroglobulin | 984 | 81.15 | 12620 |
| Coagulation | FGB | 110135 | Fibrinogen, B beta polypeptide | 37 | 61.75 | 5601 |
| | FGA | 14161 | Fibrinogen, A alpha polypeptide | 35 | 49.81 | 5549 |
| | PLG | 18815 | Plasminogen | 4 | 11.82 | 11 |
| | KLK1 | 16612 | Kallikrein B | 4 | 21.94 | 10 |
| | KNG1 | 16644 | Kininogen-1 | 4 | 18.13 | 9 |
| | F2 | 14061 | Prothrombin | 1 | 6.63 | 5 |
| | SERPIN-F2 | 18816 | Alpha-2-antiplasmin (Serpin F2) | 2 | 8.14 | 5 |
| | SERPIN-C1 | 11905 | Antithrombin (Serpin C1) | 5 | 33.98 | 27 |
| Complement | AHSG | 11625 | Alpha-2-HS-glycoprotein | 5 | 17.97 | 10 |
| system/ | C4BP | 12269 | C4b-binding protein | 3 | 22.6 | 13 |
| Immunity | F2 | 14061 | Coagulation factor II | 1 | 6.63 | 5 |
| | F12 | 58992 | Coagulation factor XII | 2 | 10.22 | 3 |
| | F13A1 | 74145 | Coagulation factor XIII A chain | 1 | 12.98 | 4 |
| | C1qA | 12259 | Complement C1q subcomponent subunit A | 2 | 44.08 | 9 |
| | H2-Q10 | 15007 | H-2 class I histocompatibility Ag, Q10 alpha chain | 2 | 12 | 8 |
| | C1qB | 12260 | Complement C1q subcomponent subunit B | 2 | 11.07 | 11 |
| | SERPIN-G1 | 12258 | Plasma protease Cl inhibitor (Serpin-G1) | 3 | 14.88 | 4 |
| | H2-T5 | 110561 | MHC class Ib T5 | 1 | 7.6 | 8 |
| | AZGP1 | 12007 | Zinc-alpha-2-glycoprotein 1 | 1 | 13.68 | 1 |
| | PIGR | 18703 | Polymeric immunoglobulin receptor | 5 | 14.92 | 28 |
| | MBL1 | 17194 | Mannose-binding lectin-1 | 2 | 10.25 | 3 |
| | FCN1 | 14133 | Ficolin-1 | 2 | 13.47 | 2 |
| | C2 | 12263 | Complement C2 | 4. | 12.07 | 14 |
| | C5 | 15139 | Complement C5 | 15 | 22.38 | 47 |
| | CFb | 14962 | Complement factor B | 4 | 23.98 | 14 |
| | CFh | 12628 | Complement factor H | 8 | 20.26 | 26 |
| | Cfi | 12630 | Complement factor I | 4 | 9.78 | 8 |
| | C4b | 12268 | Complement C4-B | 25 | 31.76 | 88 |
| | C8g | 69379 | Complement C8, gamma subunit | 1 | 39.29 | 4 |
| | C9 | 12279 | Complement C9 | 1 | 2.19 | 1 |
| | C8b | 110382 | Complement C8, beta subunit | 1 | 9.5 | 4 |
| | CRP | 12944 | C-reactive protein | 2 | 20.89 | 4 |
| | C3 | 12266 | Complement C3 | 59.47 | 94 | 446 |
| Apoptosis | CTSD | 13033 | Cathepsin-D | 1 | 7.86 | 1 |
| | CLU | 12759 | Clusterin | 6 | 15.85 | 22 |
| | CD5L | 11801 | CD5 antigen-like | 17 | 42.33 | 63 |
| Angiogenesis/ | ECM1 | 13601 | Extracellular matrix protein 1 | 1 | 10.55 | 3 |
| Cell | DSG1A | 13510 | Desmoglein 1 alpha | 1 | 1.22 | 4 |
| adhesion | SHC1 | 20416 | SHC-transforming protein 1 | 1 | 2.85 | 1 |
| | DSG1B | 225256 | Desmoglein 1 beta | 1 | 1.22 | 1 |
| | ANXA2 | 12306 | Annexin A2 | 3 | 12.98 | 3 |
| | FN1 | 14268 | Fibronectin | 103 | 43.36 | 2773 |
| | LGALS3BP | 19039 | Galectin-3-binding protein | 1 | 4.67 | 2 |
| | VTN | 22370 | Vitronectin | 3 | 10.46 | 5 |

^{a} Significant MS/MS number of peptides identified in blood pure extracellular vesicles from CDAA-fed mice.
^{b} Significant MS/MS absolute % of coverage calculated in blood pure extracellular vesicles from CDAA-fed mice.
^{c} Significant MS/MS spectral counts identified in blood pure extracellular vesicles from CDAA-fed mice.

TABLE 4
Proteins identified in the CSAA-fed mice
circulating extracellular vesicles by LC-MS/MS

| Gene | | Peptides | |
|---|---|---|---|
| symbol | Protein deseription | (95%)^{a} | % Cov^{b} |
| IGHG1 | Ig gamma-1 chain C region secreted | 43 | 58.3 |
| | form | | |
| A2A513 | Keratin 10 | 22 | 25.3 |
| K1C10 | Keratin, type I cytoskeletal 10 | 22 | 24.9 |
| K1C10 | Isoform 3 of Keratin, type I cytoskeletal | 22 | 30.7 |
| | 10 | | |
| K1C10 | Isoform 2 of Keratin, type I cytoskeletal | 22 | 25.3 |
| | 10 | | |
| Q32PO4 | Keratin 5 | 8 | 15.5 |
| K2C5 | Keratin, type II cytoskeletal 5 | 8 | 15.5 |
| IGKC | Ig kappa chain C region | 34 | 63.2 |
| Q546G4 | Albumin 1 | 6 | 10.2 |
| ALBU | Serum albumin | 6 | 10.2 |
| K2C1 | Keratin, type II cytoskeletal 1 | 17 | 8.3 |
| KV2A7 | Ig kappa chain V-II region 26-10 | 12 | 46.9 |
| K22E | Keratin, type II cytoskeletal 2 epidermal | 7 | 13.6 |
| K2C73 | Keratin, type II cytoskeletal 73 | 9 | 15.2 |
| Q3ZAW8 | Keratin 16 | 3 | 11.1 |
| K1C16 | Keratin, type I cytoskeletal 16 | 3 | 11.1 |
| Q4KL81 | Actin, gamma, cytoplasmic 1 | 2 | 9.1 |
| B2RRX1 | Actin, beta | 2 | 9.1 |
| ACTG | Actin, cytoplasmic 2 | 2 | 9.1 |
| ACTB | Actin, cytoplasmic 1 | 2 | 9.1 |
| ACTBL | Beta-actin-like protein 2 | 1 | 9.0 |
| Q497E4 | Actin alpha cardiac | 1 | 9.0 |
| Q3UJ36 | Actin, gamma 2, smooth muscle, enteric, | 1 | 9.0 |
| | isoform | | |
| ACTS | Actin, alpha skeletal muscle | 1 | 9.0 |
| ACTC | Actin, alpha cardiac muscle 1 | 1 | 9.0 |
| ACTH | Actin, gamma-enteric smooth muscle | 1 | 9.0 |
| ACTA | Actin, aortic smooth muscle | 1 | 9.0 |
| B1ATY0 | Actin, gamma, cytoplasmic 1 | 1 | 5.4 |
| MUCM | Ig mu chain C region membrane-bound | 2 | 5.9 |
| | form | | |
| IGHM | Ig mu chain C region secreted form | 2 | 6.2 |
| K1C17 | Keratin, type I cytoskeletal 17 | 6 | 27.0 |
| K22O | Keratin, type II cytoskeletal 2 oral | 5 | 14.3 |
| PLK4 | Serine/threonine-protein kinase PLK4 | 1 | 2.7 |
| PLK4 | Isoform 3 of Serine/threonine-protein | 1 | 2.8 |
| | kinase PLK4 | | |
| PLK4 | Isoform 2 of Serine/threonine-protein | 1 | 5.4 |
| | kinase PLK4 | | |
| Q08EK5 | Keratin 77 | 12 | 10.3 |
| K2C1B | Keratin, type II cytoskeletal 1b | 12 | 10.3 |
| REFP2 | RNA and export factor-binding protein 2 | 1 | 8.3 |
| REFP2 | Isoform 2 of RNA and export factor- | 1 | 8.3 |
| | binding protein | | |
| THOC4 | THO complex subunit 4 | 1 | 7.1 |
| THOC4 | Isoform 2 of THO complex subunit 4 | 1 | 11.0 |
| Q3V3K3 | Putative uncharacterized protein | 1 | 2.3 |
| TAOK3 | Serine/threonine-protein kinase TAO3 | 1 | 1.1 |

^{a}Significant MS/MS number of peptides identified in blood pure extracellular vesicles from CSAA-fed mice;
^{b}Significant MS/MS absolute % of coverage calculated in blood pure extracellular vesicles from CSAA-fed mice

Circulating EVs are Released in a Time-Dependent Manner and Correlate with Histological Features of Liver Damage

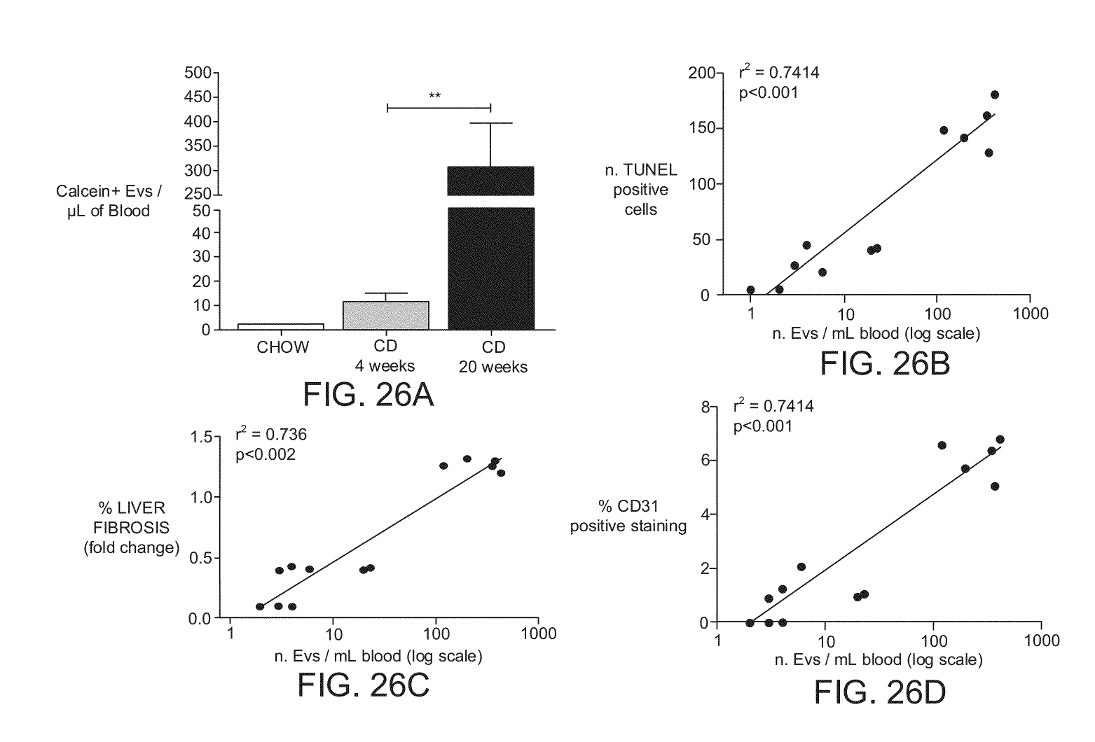

The findings of increased levels of circulating EVs in mice with established severe NASH lead to further examine the potential role of these vesicles for noninvasive monitoring of disease progression over time and histological severity. In order to address these questions, male C57BL/6 mice were placed on the CDAA, CSAA or a regular Chow diet for 4 and 20 weeks. These time points were chosen because they have been shown to be associated with early stage and established NASH, respectively [28]. It was observed that the increase in circulating EVs isolated from platelet-free plasma was time-dependent (FIG. 26A).

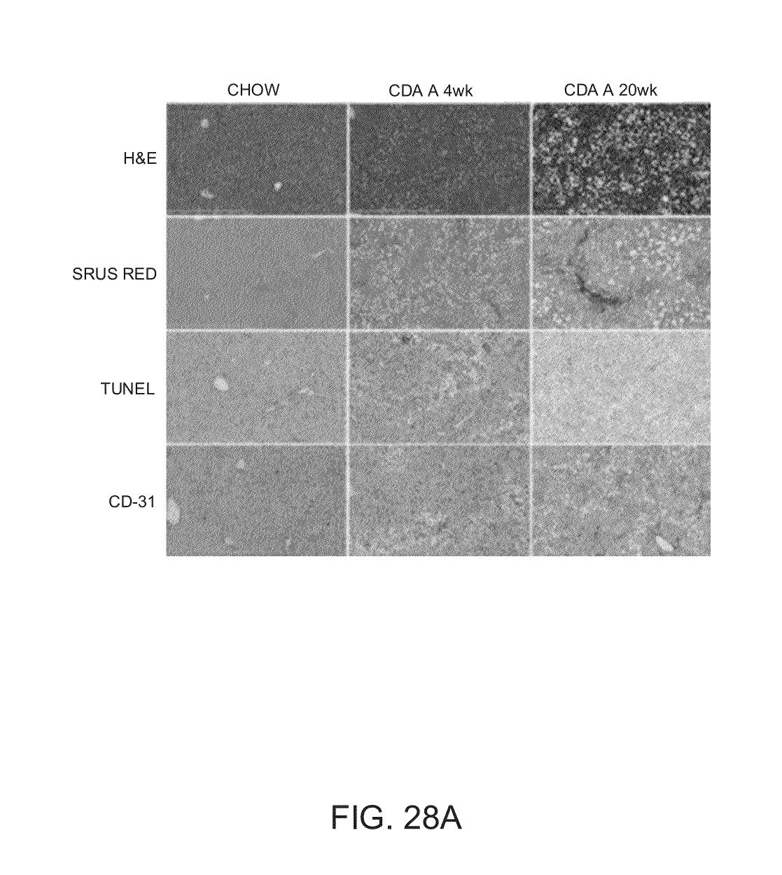

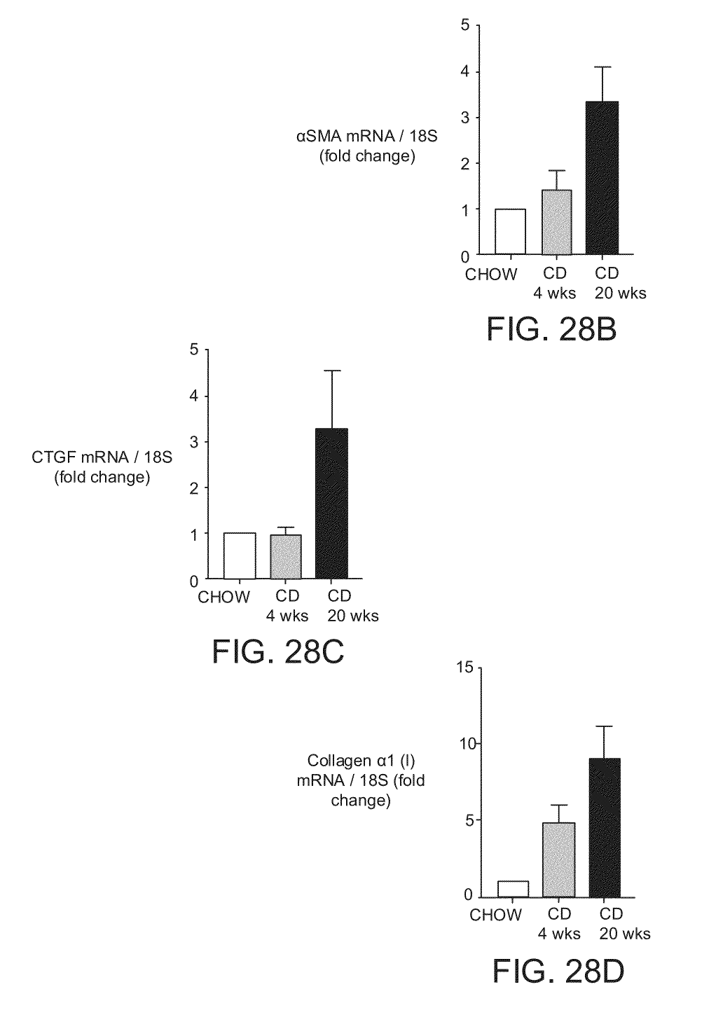

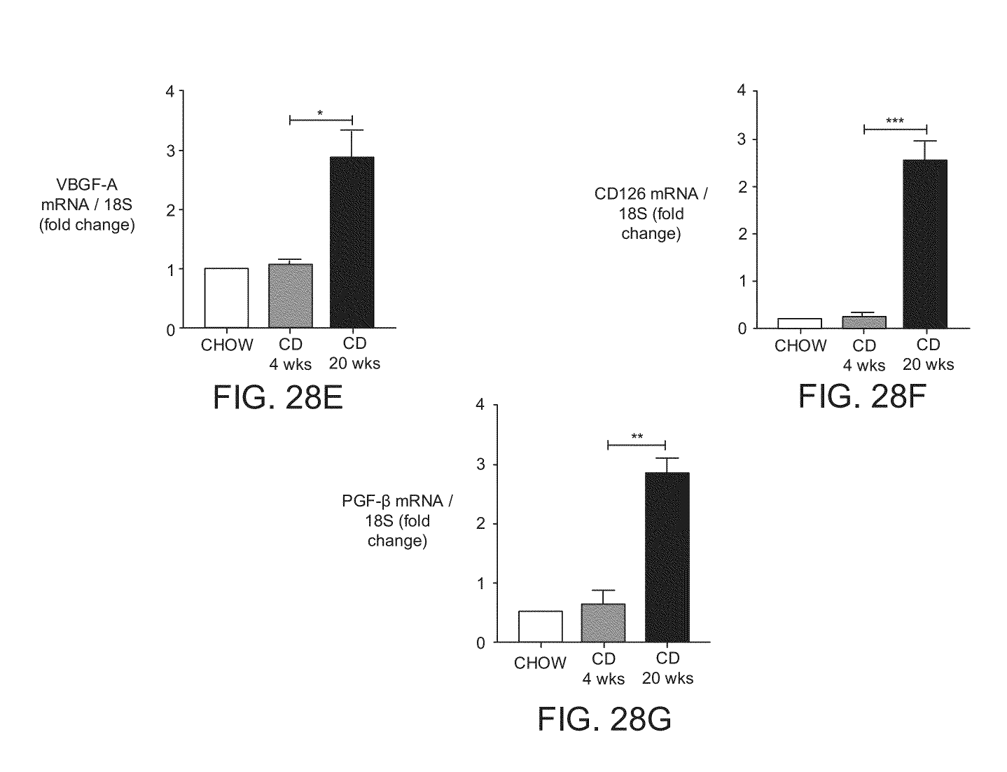

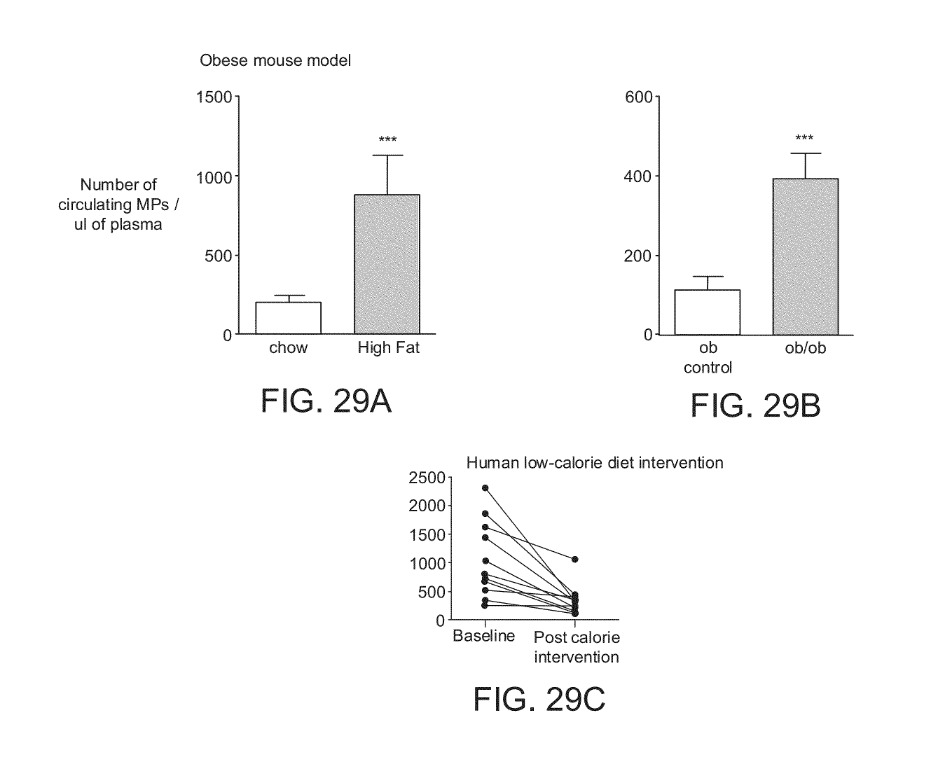

Determination of liver damage and fibrosis was further assessed by morphologic quantification of collagen deposition by Sirius red staining, apoptotic cells by TUNEL staining and neovessel formation by an immunostaining analysis of a specific marker of endothelial cells forming neovessels (CD-31). Mice placed on the CDAA diet for 20 weeks developed severe cell death, hepatic fibrosis and pathological angiogenesis, while mice on the CDAA diet for 4 weeks showed only signs of early disease, mainly isolated hepatic steatosis (FIG. 28A). These findings were also confirmed by the analysis of the transcripts for the pro-angiogenic and pro-fibrogenic genes (FIG. 28B), indicating a time-dependent progression of all the main features of liver injury associated with NASH. It was found that the number of circulating MPs were increased in mouse models of NAFLD, either wild type fed with a high fat diet or leptin deficient (ob/ob) mouse (FIG. 29A, *** p<0.001); but the number of circulating MPs were decreased when patients with NAFLD were placed on a low calorie diet for 3 months (FIG. 29B).

Correlation analysis of the number of blood EVs with the histopathological features of NASH showed a strong correlation, especially with the amount of apoptotic cells (r₂=0.804, p<0.001), extent of liver fibrosis (r₂=0.736, p<0.002) and pathological angiogenesis (r₂=0.741, p<0.001) (FIGS. 26B-D). These findings suggest that blood MPs are released during NASH development in a time-dependent manner and their levels strongly correlate with the extent of liver damage.

### DISCUSSION

The main findings of this example relate to the role of EVs as potential biomarkers of liver damage in NASH. The results demonstrate that well established NASH induced by feeding mice a CDAA diet for 20 weeks is associated with increased production and release of EVs in the liver and in circulation. Detailed characterization of these vesicles identified both exosomes and microparticles, with the latter being the main population of vesicle released into the bloodstream during the diet-induced NASH. Moreover, blood EVs levels were dynamic, increasing over time and closely correlated with key histopathological features of disease severity. In light of the dramatic increase in the prevalence of NAFLD, in conjunction with the significant research effort in developing novel therapies targeted to patients with NASH, dynamic, noninvasive biomarkers that can be measured periodically to track disease changes in real time are in great need. Biomarkers would not only help in the diagnosis of NASH, but also be useful for the assessment of treatment response and prognosis. The study demonstrates that EVs—membrane surrounded structures released from a variety of cell types during stress or apoptosis and have been growingly linked to cell-cell communication—have the potential to fulfill these requirements. Indeed, recent studies have demonstrated that primary murine hepatocytes, as well as different hepatocyte cell lines, are capable of releasing the two main subtypes of extracellular vesicles: exosomes and MPs [18, 20, 21].

Moreover, in vivo studies in bile duct-ligated rats have found increased circulating MPs, while two recent pilot studies in humans showed increased levels of inflammatory cell derived MPs in patients with NAFLD and in patients with alcohol and/or chronic hepatitis C related cirrhosis [23]. The data presented herewith demonstrated that the 20 week CDAA diet course resembles human established NASH. Notably, mice fed the CDAA diet developed massive macrovesicular hepatic steatosis, fibrosis, cell death and pathological angiogenesis compared to the mice that received the control diets. Moreover, the 20 week CDAA-fed mice showed a massive production and release of extracellular vesicles in the bloodstream and in the liver.

Detailed characterization of the blood EVs demonstrated that exosomes and microparticles were both present in the vesicles population. Proteomic analysis of blood EVs isolated from mice fed with the CDAA diet for 20 weeks showed a number of proteins that were not present in mice fed the control CSAA diet. While many of these proteins were found in previous reports from hepatocyte-derived EVs in vitro and in human blood, others were not previously detected [21, 29, 30]. Interestingly, significant number of proteins involved in cell death, angiogenesis and related cell-adhesion, antioxidant and redox signaling and inflammatory response processes were found. The enrichment of these proteins in the circulating EVs released during NASH suggests that these vesicles carry specific signatures, and materials, reflecting the pathological condition of the organism from which they have been isolated. Annotations of the GO Consortium were used to connect the variety of proteins found in the circulating EVs to the biological processes and molecular function. Remarkably, a large number of proteins were associated with the inflammatory and immunity machinery, including proteins involved in the complement system pathway and its regulation.

The importance of the EVs as an efficient vehicle for different molecules is demonstrated by the presence, in the EV proteome, of a large number of proteins secreted by different cells, but particularly by hepatocytes. The presence of oxidative stress and production of reactive oxygen species (ROS) by dysfunctional mitochondria as a consequence of hepatocyte lipotoxicity during NAFLD progression [31] could explain the significant presence of proteins involved in the redox signaling regulation in the cells. Indeed, several antioxidant enzymes—proteins involved in the response to stress and protection from oxidative damage were identified. Importantly, a number of proteins noteworthy for their involvement in cell death, which is a key process involved in liver injury and NASH development [32] was also detected. In particular proteins that have serine/cysteine protease inhibitor domains [33] were found. The presence of lysosomal proteases involved in lysosomal permeabilization, as well as apoptosis promoter antigens, represent an additional sign of adipocyte death and macrophage infiltration associated with insulin resistance and NASH [34].

The presence of a large number of proteins involved in angiogenesis was also identified in the EVs proteome, which included proteins that play a role in cell motility, cell-cell adhesion, migration, sprouting and neovessel formation, processes that appear to be important mechanisms in disease progression [35]. The proteome of circulating EVs of CDAA-fed mice also reflects the mechanisms of vesicle formation, as demonstrated by the presence of glycolytic enzymes, cytoskeleton structural proteins and GTPases, which play a role in the calcium-dependent or independent vesicle formation and trafficking processes. The presence of atherogenic lipoproteins and scavenger receptors could suggest a strong link between non-alcoholic steatohepatitis and cardiovascular complications, as have been previously described [36].

Based on these results, the potential role of the circulating extracellular vesicles increased during NASH for noninvasive monitoring of disease progression over time and histological severity were further examined in mice fed with the CDAA diet for 4 weeks (early NASH) or 20 weeks (established NASH). Mice placed on a CDAA diet for 20 weeks developed severe cell death, hepatic fibrosis and pathological angiogenesis, while mice on the CDAA diet for only 4 weeks showed signs of early disease—mainly isolated hepatic steatosis. The levels of circulating extracellular vesicles isolated from CDAA-fed mice for 4 weeks and 20 weeks were time-dependent and strongly correlated with the histopathological features of NASH. In particular, the level of circulating EVs released during the diet-induced NASH strongly correlated with hepatic fibrosis, cell death and pathological angiogenesis. Thus, these findings show that extracellular vesicles are produced and released during NASH and have a specific antigenic composition reflecting the pathological alterations typical of NAFLD progression. Additionally, the EV levels are dynamic and change over time, correlating with changes in liver histopathology characteristic of NASH.

In conclusion, the studies presented in this example suggest that monitoring EVs in circulation is a novel non-invasive tool to assess disease progression in NAFLD, and uncover the antigenic profile of these vesicles during murine NASH development, which are potential targets for immune-based diagnostics development.

### Materials and Methods

### Animal Studies

C57BL/6 wild type mice, 20 to 25 gm of body weight, 7 weeks old, were placed on a Choline Deficient L-Amino Acid (CDAA) (Dyets, Bethlehem, Pa., USA) diet and a control diet (Choline Supplemented L-Amino Acid, CSAA) diet for 4 weeks and 20 weeks. This diet has been extensively shown to result in steatosis associated with significant inflammation and progressive fibrosis, pathologically similar to human severe steatohepatitis [28, 37, 38]. Mice were sacrificed and liver and blood were collected under deep anesthesia obtained by injecting intraperitoneally a mixture of 100 mg/Kg of Ketamine and 10 mg/Kg of Xylazine dissolved in a saline solution. For the injection a 21 G needle has been used [39]. Liver samples were preserved differently depending on the purpose (RNA isolation, protein isolation, cryopreservation and paraffin-embedding). The studies were approved by the University of California San Diego Institutional Animal Care and Use Committee and followed the National Institutes of Health guidelines outlined in “Guide for the Care and Use of Laboratory Animals”.

### Histopathology and Immunohistochemistry

Liver tissue was fixed in 10% formalin up to 24 hours. After a quick wash with running tap water, tissue was paraffin embedded. Tissue sections (10 μm) were prepared, stained for hematoxylin and eosin (H&E) and examined in a blinded fashion by a single experienced pathologist to determine histological changes including degree of steatosis, fibrosis and inflammation under light microscopy [40]. The presence of tissue neovessels with tube-like formation was assessed by immunostaining for polyclonal rabbit antibody anti-CD-31 (1:25; Abcam, Cambridge, Mass.). Slides were de-paraffinized by several passages in xylene and different % of ethanol, including a final washing with phosphate buffer saline and distilled water. De-paraffinized sections were immersed in 3% H2O2 in water for 15 minutes to eliminate the endogenous peroxidase activity. Sections were processed for heat-induced epitope retrieval for 20 minutes by using Dako Target Retrieval Solution pH 6.0 (Dako, Glostrup, Denmark) and stained overnight at 4° C. After the incubation with the secondary antibody, immune complexes were detected by using DakoEnVision with HRP system (Dako, Glostrup, Denmark), according to the manufacturer's instructions. Quantification of the CD-31 positive staining was performed by the image analysis algorithm designed and provided by Wimasis GmbH (Munich, Germany). For detection of tissue collagen deposition, a routine Sirius red (saturated picric acid containing 0.1% Direct Red 80 and 0.1% Fast Green FCF) staining was performed. The Sirius red positive area was calculated in five random low power views on each slide using Image J Software (NIH). ApopTag peroxidase in situ apoptosis detection kit (Millipore, Billerica, Mass., USA) was used according to the manufacturer, to detect cell death in paraffin-embedded liver specimens. Positive cells were counted in different fields. A 10× magnification was used.

### Isolation of Circulating Extracellular Vesicles

Circulating extracellular vesicles were isolated from fresh blood samples harvested from CDAA, CSAA and chow fed mice for 4 and 20 weeks. Approximately 1 mL of whole blood was collected in heparin-conditioned 1.5 mL tubes and centrifuged at 1,200 g for 15 minutes in order to obtain platelet-poor plasma (PPP). Supernatant containing EVs was centrifuged at 12,000 g for 12 minutes in order to get the platelet-free plasma (PFP). To determine size and protein expression of both microparticles and exosomes, PFP was additionally ultracentrifuged at 20,000 g for 30 minutes at 10° C. (SW41, Beckman, Indianapolis, Ind., USA) to pellet the microparticles. The MP-free supernatant was transferred in new tubes and ultracentrifuged at 100,000 g for 1 h at 10° C. to pellet the exosomes. The size of MPs and exosomes was determined by Dynamic Light Scattering Zetasizer (Malvern, Worcestershire, UK). For the proteomics analysis by LC-MS/MS, PFP was ultracentrifuged at 100,000 g for 1 h at 10° C. to pellet the whole EVs, which have been further purified by using a 10-70% sucrose gradient, resulting in densities ranging 1.07-1.25 g/mL. Samples were ultracentrifuged at 100,000 g for 18 h at 10° C. Different fractions were collected, resuspended in PBS and further ultracentrifuged at 100,000 g for 1 h at 10° C. The resulted purified EVs preps were processed for the proteomics analysis.

### Flow Cytometry

Circulating extracellular vesicle acquisition was performed by means of the BD LSRII Flow Cytometer System (BD Biosciences, San Jose, Calif., USA) and the data were analyzed using FlowJo software (TreeStar Inc., Ashland, Oreg., USA). A volume of 30 μL of PFP was incubated with 1 μM of Calcein AM (BD Biosciences, San Jose, Calif., USA) in PBS for 1 h at 37° C. Calcein AM is a cell-permeant dye that is converted to a green-fluorescent Calcein (emission wavelength of 515 nm) after acetoxymethyl ester hydrolysis by intracellular esterases. Standardization of the protocol was achieved using 1 μm latex fluorescent beads (Sigma-Aldrich, St Louis, Mo., USA) and ultraviolet 2.5 μm flow cytometry alignment beads (Invitrogen, Grand Island, N.Y., USA). Forward (FS) and side scatter (SS) parameters were plotted on logarithmic scales to best cover a wide size range. Single staining controls were used to check fluorescence compensation settings and to set up positive regions.

### Sample Preparation for MS

Protein samples were diluted in THE (50 mM Tris pH 8.0, 100 mM NaCl, 1 mM EDTA) buffer. RapiGest SF reagent (Waters Corp.) was added to the mix to a final concentration of 0.1% and samples were boiled for 5 min. TCEP (Tris(2-carboxyethyl) phosphine) was added to 1 mM (final concentration) and the samples were incubated at 37° C. for 30 min. Subsequently, the samples were carboxymethylated with 0.5 mg/ml of iodoacetamide for 30 min at 37° C. followed by neutralization with 2 mM TCEP (final concentration). Protein samples prepared as above were digested with trypsin (trypsin:protein ratio—1:50) overnight at 37° C. RapiGest was degraded and removed by treating the samples with 250 mM HCl at 37° C. for 1 h followed by centrifugation at 14000 rpm for 30 min at 4° C. The soluble fraction was then added to a new tube and the peptides were extracted and desalted using Aspire RP30 desalting columns (Thermo Scientific).

### LC-MS-MS Analysis

Trypsin-digested peptides were analyzed by high pressure liquid chromatography (HPLC) coupled with tandem mass spectroscopy (LC-MS/MS) [41]. The nanospray ionization experiments were performed using a TripleT of 5600 hybrid mass spectrometer (ABSCIEX) interfaced with nano-scale reversed-phase HPLC (Tempo) using a 10 cm-100 micron ID glass capillary packed with 5-μm C18 Zorbax™ beads (Agilent Technologies, Santa Clara, Calif.). Peptides were eluted from the C18 column into the mass spectrometer using a linear gradient (5-60%) of ACN (Acetonitrile) at a flow rate of 2500 min for 1 h. The buffers used to create the ACN gradient were: Buffer A (98% H2O, 2% ACN, 0.2% formic acid, and 0.005% TFA) and Buffer B (100% ACN, 0.2% formic acid, and 0.005% TFA). MS/MS data were acquired in a data-dependent manner in which the MS1 data were acquired for 250 ms at m/z of 400 to 1250 Da and the MS/MS data were acquired from m/z of 50 to 2,000 Da. For Independent data acquisition (IDA) parameters MS1-TOF 250 milliseconds, followed by 50 MS2 events of 25 milliseconds each. The IDA criteria, over 200 counts threshold, charge state +2-4 with 4 seconds exclusion. Finally, the collected data were analyzed using MASCOT® (Matrix Sciences) and Protein Pilot 4.0 (ABSCIEX) for peptide identifications [42, 43].

### Electron Microscopy

For transmission electron microscope, extracellular vesicles were adhered to 100 mesh Formvar and carbon coated grids for 5 minutes at room temperature. Grids were washed once with water, stained with 1% uranyl acetate (Ladd Research Industries, Williston Vt.) for 1 minute, dried and viewed using a JEOL 1200 EXII transmission electron microscope. Images were captured using a Gatan Orius 600 digital camera (Gatan, Pleasanton, Calif.). Liver samples were collected from the CDAA-fed mice after a short liver perfusion with 10 mL of 4% paraformaldehyde in 0.15 M sodium cacodylate buffer, pH 7.4 by using a 21 G needle. Samples were immersed in modified Karnovsky's fixative (2.5% glutaraldehyde and 2% paraformaldehyde in 0.15 M sodium cacodylate buffer, pH 7.4) for at least 4 hours, post fixed in 1% osmium tetroxide in 0.15 M cacodylate buffer for 1 hour and stained en bloc in 3% uranyl acetate for 1 hour. Samples were dehydrated in ethanol, embedded in Durcupan epoxy resin (Sigma-Aldrich), sectioned at 50 to 60 nm on a Leica UCT ultramicrotome, and picked up on Formvar and carbon-coated copper grids. Sections were stained with 3% uranyl acetate for 5 minutes and Sato's lead stain for 1 minute. Grids were viewed using a JEOL 1200EX II (JEOL, Peabody, Mass.) transmission electron microscope and photographed using a Gatan digital camera (Gatan, Pleasanton, Calif.).

### Real-Time PCR

Liver tissue from C57BL/6 mice fed with CDAA, CSAA or chow diet, were homogenized using the FastPrep 24 bead homogenization system. Total RNA was isolated using RNeasy kit (Qiagen, Valencia, Calif.) and reverse transcribed by iScript cDNA synthesis kit (Bio-Rad) according to the manufacturer instructions. The concentration and purity of RNA was assessed by NanoDrop (Thermo Scientific). Quantitative Real time PCR was performed on a BioRad Cycler (Biorad, Hercules, Calif., USA) by using SYBRGreen real time PCR master mix (Kapabiosystem, Woburn, Mass., USA) according to the manufacturer instructions. The housekeeping gene 18S was used as an internal control.

### Western Blots

Extracellular vesicles were isolated from blood samples collected from the animals, as described in the MPs isolation and purification” paragraph. Approximately 10 μg of pure EVs protein lysates were solubilized in Laemli buffer, resolved by a 4-20% Criterion Tris-HCl gel electrophoresis (Biorad, Hercules, Calif., USA) and transferred to a 0.2 μm nitrocellulose membrane (Biorad, Hercules, Calif., USA). Membranes were blocked for 1 hour at room temperature with 3-5% low-fat milk (Biorad, Hercules, Calif., USA) in TBS, 0.05% Tween 20 (TBS-T). Primary rabbit polyclonal antibody anti-mouse Vanin-1 (1:500; Proteintech, Chicago, Ill., USA), Cd63, Cd81 (1:1000; Genetex, Irvine, Calif., USA) and Icam-1 (1:1000; Abnova, Taipei city, Taiwan) were incubated overnight at 4° C. After washing with PBS-T, membranes were incubated with goat anti-rabbit secondary antibody. Proteins were visualized by Supersignal West Pico chemiluminescence substrate (Pierce biotechnology, Rockford, Ill., USA) and quantified by ImageJ software.

### Statistical Analysis

All data were expressed as the mean±SD unless otherwise indicated. Differences between three or more groups were compared by an ANOVA analysis followed by a post-hoc Bonferroni test. Differences between two groups of normalized data were compared by a two-tailed Student's t-test. Differences were considered to be statistically significant at P<0.05. Pearson's correlation coefficients were calculated for the correlation analyses and an r₂ value higher than 0.7 was considered a strong correlation. All statistical analyses were performed using GraphPad Prism 4.0c (La Jolla, Calif., USA).

### REFERENCES

1. Williams C D, Stengel J, Asike M I, Torres D M, Shaw J, Contreras M, et al. Prevalence of nonalcoholic fatty liver disease and nonalcoholic steatohepatitis among a largely middle-aged population utilizing ultrasound and liver biopsy: a prospective study. Gastroenterology 2011; 140:124-31.
2. Nanda K. Non-alcoholic steatohepatitis in children. Pediatr Transplant 2004; 8:613-8.
3. Bambha K, Belt P, Abraham M, Wilson L A, Pabst M, Ferrell L, et al. Ethnicity and nonalcoholic fatty liver disease. Hepatology 2012; 55:769-80.
4. Alfire M E, Treem W R. Nonalcoholic fatty liver disease. Pediatr Ann 2006; 35:290-4, 297-9.
5. Angulo P. Nonalcoholic fatty liver disease. N Engl J Med 2002; 346:1221-31.
6. Alkhouri N, Dixon L J, Feldstein A E. Lipotoxicity in nonalcoholic fatty liver disease: not all lipids are created equal. Expert Rev Gastroenterol Hepatol 2009; 3:445-51.
7. Malhi H, Gores G J. Molecular mechanisms of lipotoxicity in nonalcoholic fatty liver disease. Semin Liver Dis 2008; 28:360-9.
8. Liou I, Kowdley K V. Natural history of nonalcoholic steatohepatitis. J Clin Gastroenterol 2006; 40 Suppl 1:S11-6.
9. Wieckowska A, McCullough A J, Feldstein A E. Noninvasive diagnosis and monitoring of nonalcoholic steatohepatitis: present and future. Hepatology 2007; 46:582-9.
10. Torres D M, Harrison S A. Diagnosis and therapy of nonalcoholic steatohepatitis. Gastroenterology 2008; 134:1682-98.
11. Gyorgy B, Szabo T G, Pasztoi M, Pal Z, Misjak P, Aradi B, et al. Membrane vesicles, current state-of-the-art: emerging role of extracellular vesicles. Cell Mol Life Sci 2011; 68:2667-88.
12. Kornek M, Schuppan D. Microparticles: Modulators and biomarkers of liver disease. J Hepatol 2012; 57:1144-6.
13. Diehl P, Fricke A, Sander L, Stamm J, Bassler N, Htun N, et al. Microparticles: major transport vehicles for distinct microRNAs in circulation. Cardiovascular research 2012; 93:633-44.
14. Yuan A, Farber E L, Rapoport A L, Tejada D, Deniskin R, Akhmedov N B, et al. Transfer of microRNAs by embryonic stem cell microvesicles. PloS one 2009; 4:e4722.
15. Raposo G, Stoorvogel W. Extracellular vesicles: exosomes, microvesicles, and friends. J Cell Biol 2013; 200:373-83.
16. Morel O, Morel N, Jesel L, Freyssinet J M, Toti F. Microparticles: a critical component in the nexus between inflammation, immunity, and thrombosis. Seminars in immunopathology 2011; 33:469-86.
17. Rautou P E, Vion A C, Amabile N, Chironi G, Simon A, Tedgui A, et al. Microparticles, vascular function, and atherothrombosis. Circulation research 2011; 109:593-606.
18. Witek R P, Yang L, Liu R, Jung Y, Omenetti A, Syn W K, et al. Liver cell-derived microparticles activate hedgehog signaling and alter gene expression in hepatic endothelial cells. Gastroenterology 2009; 136:320-330 e2.
19. Povero D, Eguchi A, Lazic M, Johnson C, Parola M, Feldstein A E. Hepatocytes release microparticles during lipotoxicity that have potent pro-angiogenic effects in vitro and in vivo through Vanin-1-dependent internalization. Hepatology 2012; 56:251A.
20. Pan Q, Ramakrishnaiah V, Henry S, Fouraschen S, de Ruiter P E, Kwekkeboom J, et al. Hepatic cell-to-cell transmission of small silencing RNA can extend the therapeutic reach of RNA interference (RNAi). Gut 2012; 61:1330-9.
21. Conde-Vancells J, Rodriguez-Suarez E, Embade N, Gil D, Matthiesen R, Valle M, et al. Characterization and comprehensive proteome profiling of exosomes secreted by hepatocytes. J Proteome Res 2008; 7:5157-66.
22. Schuppan D, Pinzani M. Anti-fibrotic therapy: lost in translation? J Hepatol 2012; 56 Suppl 1:S66-74.
23. Kornek M, Lynch M, Mehta S H, Lai M, Exley M, Afdhal N H, et al. Circulating microparticles as disease-specific biomarkers of severity of inflammation in patients with hepatitis C or nonalcoholic steatohepatitis. Gastroenterology 2012; 143:448-58.
24. Kornek M, Popov Y, Libermann T A, Afdhal N H, Schuppan D. Human T cell microparticles circulate in blood of hepatitis patients and induce fibrolytic activation of hepatic stellate cells. Hepatology 2011; 53:230-42.
25. Denzer K, Kleijmeer M J, Heijnen H F, Stoorvogel W, Geuze H J. Exosome: from internal vesicle of the multivesicular body to intercellular signaling device. J Cell Sci 2000; 113 Pt 19:3365-74.
26. Fevrier B, Raposo G. Exosomes: endosomal-derived vesicles shipping extracellular messages. Curr Opin Cell Biol 2004; 16:415-21.
27. Creating the gene ontology resource: design and implementation. Genome Res 2001; 11:1425-33.
28. Miura K, Yang L, van Rooijen N, Ohnishi H, Seki E. Hepatic recruitment of macrophages promotes nonalcoholic steatohepatitis through CCR2. Am J Physiol Gastrointest Liver Physiol 2012; 302:G1310-21.
29. Ostergaard O, Nielsen C T, Iversen L V, Jacobsen S, Tanassi J T, Heegaard N H. Quantitative proteome profiling of normal human circulating microparticles. J Proteome Res 2012; 11:2154-63.
30. Capriotti A L, Caruso G, Cavaliere C, Piovesana S, Samperi R, Lagana A. Proteomic characterization of human platelet-derived microparticles. Anal Chim Acta 2013; 776:57-63.
31. Browning J D, Horton J D. Molecular mediators of hepatic steatosis and liver injury. J Clin Invest 2004; 114:147-52.
32. Ibrahim S H, Kohli R, Gores G J. Mechanisms of lipotoxicity in NAFLD and clinical implications. J Pediatr Gastroenterol Nutr 2011; 53:131-40.
33. Richardson J, Viswanathan K, Lucas A. Serpins, the vasculature, and viral therapeutics. Front Biosci 2006; 11:1042-56.
34. Gornicka A, Fettig J, Eguchi A, Berk M P, Thapaliya S, Dixon L J, et al. Adipocyte hypertrophy is associated with lysosomal permeability both in vivo and in vitro: role in adipose tissue inflammation. Am J Physiol Endocrinol Metab 2012; 303:E597-606.
35. Kitade M, Yoshiji H, Noguchi R, Ikenaka Y, Kaji K, Shirai Y, et al. Crosstalk between angiogenesis, cytokeratin-18, and insulin resistance in the progression of non-alcoholic steatohepatitis. World J Gastroenterol 2009; 15:5193-9.
36. Hallsworth K, Hollingsworth K G, Thoma C, Jakovljevic D, Macgowan G A, Anstee Q M, et al. Cardiac structure and function are altered in adults with non-alcoholic fatty liver disease. J Hepatol. 2012.
37. Denda A, Kitayama W, Kishida H, Murata N, Tsutsumi M, Tsujiuchi T, et al. Development of hepatocellular adenomas and carcinomas associated with fibrosis in C57BL/6J male mice given a choline-deficient, L-amino acid-defined diet. Jpn J Cancer Res 2002; 93:125-32.
38. Kodama Y, Kisseleva T, Iwaisako K, Miura K, Taura K, De Minicis S, et al. c-Jun N-terminal kinase-1 from hematopoietic cells mediates progression from hepatic steatosis to steatohepatitis and fibrosis in mice. Gastroenterology 2009; 137:1467-1477 e5.
39. Li Z, Berk M, McIntyre T M, Gores G J, Feldstein A E. The lysosomal-mitochondrial axis in free fatty acid-induced hepatic lipotoxicity. Hepatology 2008; 47:1495-503.
40. Kleiner D E, Brunt E M, Van Natta M, Behling C, Contos M J, Cummings O W, et al. Design and validation of a histological scoring system for nonalcoholic fatty liver disease. Hepatology 2005; 41:1313-21.
41. McCormack A L, Schieltz D M, Goode B, Yang S, Barnes G, Drubin D, et al. Direct analysis and identification of proteins in mixtures by LC/MS/MS and database searching at the low-femtomole level. Anal Chem 1997; 69:767-76.
42. Bruand J, Alexandrov T, Sistla S, Wisztorski M, Meriaux C, Becker M, et al. AMASS:algorithm for MSI analysis by semi-supervised segmentation. J Proteome Res 2011; 10:4734-43.
43. Bruand J, Sistla S, Meriaux C, Dorrestein P C, Gaasterland T, Ghassemian M, et al. Automated querying and identification of novel peptides using MALDI mass spectrometric imaging. J Proteome Res 2011; 10:1915-28.

### Example 3

### Vanin-1 Levels in NASH Patients Who Received One Year Treatment with PTX Vs. PLC

Patients cohort: 55 subjects with NASH (diagnosed by liver biopsy) which received 1-year treatment with PTX or PLC.

Inclusion criteria: 1) daily alcohol intake of <30 g for males and <15 for females; 2) appropriate exclusion for other liver diseases; 3) age between 18 and 70 years; 4) ability to provide informed consent.

Methods: EVs were purified from plasma and levels of Vanin-1 was performed by immunoassay (Mybio source, San Diego, Calif.) by using 100 uL from each sample in duplicate.

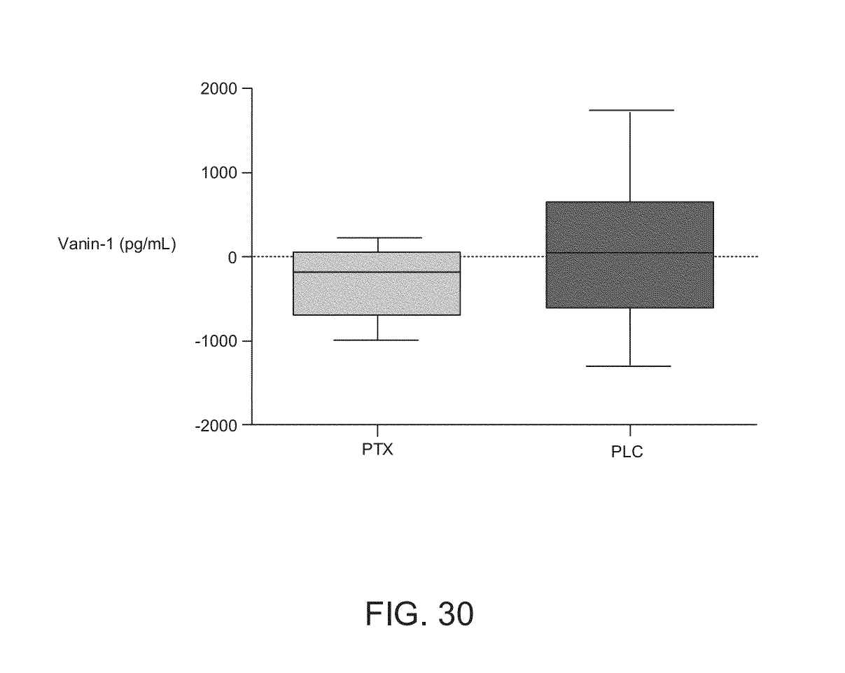

FIG. 30 illustrates a change from baseline after 1 year of therapy with Pentoxifylline (PTX) vs. placebo (PLC) in levels of Vanin-1 (pg/mL). In the box-and-whisker plot the lower boundary indicates the 25^{th} percentile, the line within the box indicates the median value and the upper boundary of the box indicates the 75^{th} percentile. The whiskers extend to the most extreme data points.

What is claimed is:

1. A method of detecting nonalcoholic fatty acid liver disease (NAFLD) in a subject, comprising:
a) obtaining a biological sample of the subject,
b) measuring circulating extracellular vesicles (EVs) in the sample, and
c) deriving a risk score for liver damage by calculating an amount of circulating EVs in the sample relative to circulating EVs in a control dataset from a population of individuals without NAFLD or liver damage associated with NAFLD, wherein an increase in circulating EVs in the subject compared to the control database indicates NAFLD detection.

2. The method of claim 1, wherein said circulating EVs are microparticles (MPs) derived from hepatocytes.

3. The method of claim 2, wherein at least one biomarker involved in molecular function and cellular localization is measured in circulating EVs or MPs.

4. The method of claim 3, wherein said biomarkers are listed in Tables 1-4.

5. The method of claim 4, where said biomarker is involved in caspase 3 activation.

6. The method of claim 2, wherein said biomarker is Vanin-1.

7. The method of claim 1, wherein said bodily sample is selected from the group consisting of blood, plasma, and serum.

8. The method of claim 1, wherein the Non-Alcoholic Fatty Liver Disease (NAFLD) is selected from the group consisting of hepatic steatosis, nonalcoholic steatohepatitis (NASH), liver fibrosis, and liver cirrhosis.

9. The method of claim 1, wherein the deriving step further comprises algorithic inclusion of quantitative data from one or more clinical indicia including at least one of the subject's age, body mass index, and liver functions relative to the same clinical indicia from the control dataset.

10. A method of treating liver damage associated with nonalcoholic steatohepatitis (NASH) in a subject in need, comprising:
administering to said subject an angiogenesis inhibiting effective amount of a composition comprising an agent that inhibits at least one biomarker expressed in hepatocyte-derived circulating extracellular vesicles (EVs), thereby treating liver damage associated with NASH in the subject.

11. The method of claim 10, wherein said circulating EVs are microparticles (MPs).

12. The method of claim 10, wherein said biomarker is involved in caspase 3 activation.

13. The method of claim 12, wherein said biomarker is Vanin-1.

14. The method of claim 10, wherein said agent inhibits caspase 3 pathway.

15. The method of claim 14, wherein said agent comprises an anti-Vanin-1 antibody.

16. The method of claim 14, wherein said agent comprises a siRNA against the nucleic acid encoding Vanin-1 protein.

17. A method of identifying a compound that inhibits at least one biomarker expressed in circulating extracellular vesicles (EVs) or microparticles (MPs) derived from hepatocytes, comprising:
a) providing a testing system that expresses said biomarker, and
b) identifying a compound that inhibits an expression of said biomarker in said testing system.

18. The method of claim 17, wherein said biomarker is listed in Tables 1-4.

19. The method of claim 18, wherein said biomarker is Vanin-1.

20. The method of claim 17, wherein said compound blocks internalization of said EVs or MPs resulting in loss of pro-angiogenic effects of EVs or MPs so as to protect a subject from angiogenesis or liver damage.
